(19) United States
(12) Patent Application Publication
(10) Pub. No.: US 2024/0216507 A1
(43) Pub. Date: Jul. 4, 2024

(54) SARS-COV-2-SPECIFIC T CELLS AND METHODS OF TREATMENT USING THEM

(71) Applicant: CHILDREN'S NATIONAL MEDICAL CENTER, Washington, DC (US)

(72) Inventors: Catherine BOLLARD, Washington, DC (US); Michael KELLER, Washington, DC (US); Chris LAZARSKI, Washington, DC (US); Allistair ABRAHAM, Washington, DC (US); Patrick HANLEY, Washington, DC (US); Conrad Russell Y. CRUZ, Washington, DC (US)

(73) Assignee: CHILDREN'S NATIONAL MEDICAL CENTER, Washington, DC (US)

(21) Appl. No.: 17/907,180

(22) PCT Filed: Mar. 22, 2021
(86) PCT No.: PCT/US2021/023428

§ 371 Date: Sep. 23, 2022

Related U.S. Application Data
(60) Provisional application No. 63/001,133, filed on Mar. 27, 2020.
(60) Provisional application No. 63/001,139, filed on Mar. 27, 2020.
(60) Provisional application No. 63/001,162, filed on Mar. 27, 2020.

(51) Int. Cl. A61K 39/00; C12N 5/0783

(52) U.S. Cl. CPC A61K 39/4611; A61K 39/464838; C12N 5/0636; C12N 2501/2304; C12N 2501/2307

(57) ABSTRACT

The invention pertains to a method for preventing or treating SARS-CoV-2 infection by administering SARS-CoV-2 specific T cells which recognize particular peptide epitopes in SARS-CoV-2 spike (S), nucleocapsid (N), membrane, and envelope proteins.

### CROSS-REFERENCE TO RELATED APPLICATION

This application claims priority to U.S. Provisional Applications 63/001,139, 63/001,162, and 63/001,133, each of which was filed Mar. 27, 2020 and each of which is incorporated by reference in its entirety.

### GOVERNMENT SUPPORT

This work was supported by funding from the NIH, including R01HL152161-02. The United States government may have rights in this disclosure.

### STATEMENT REGARDING PRIOR DISCLOSURES BY THE INVENTOR OR A JOINT INVENTOR

M. D. Keller, et al., Blood 136(25), 2905-2917 (2020) describes related technology and is incorporated by reference for all purposes.

### REFERENCE TO A SEQUENCE LISTING

In accordance with 37 CFR § 1.52(ex5), the present specification makes reference to a Sequence Listing which is submitted electronically as a .txt file named “529691WO_ST25.txt”. The .txt file was generated on Mar. 16, 2021 and is 116 kb in size. The entire contents of the Sequence Listing are herein incorporated by reference.

### BACKGROUND OF THE INVENTION

### Field of the Invention

The present disclosure pertains to the fields of infectious disease, virology, and cellular immunology. This field encompasses SARS-CoV-2-specific T-cells, T cell compositions, therapies, diagnostics, and processes of manufacture tailored for treatment or prevention of a subject with a SARS-CoV-2 infection, such as COVID-19. The present disclosure also extends to methods of manufacturing such adoptively transferable T-cell compositions and to the generation of cryogenic banks stocked with T cells which recognize SARS-CoV-2 peptide antigens for personalized T-cell therapy.

### DESCRIPTION OF RELATED ART

Severe acute respiratory syndrome coronavirus 2 (SARS-CoV-2), a novel coronavirus first reported in December 2019 from Wuhan, China, is responsible for the ongoing pandemic of coronavirus disease 2019 (COVID-19); Zhu N, et al; China Novel Coronavirus Investigating and Research Team. A novel coronavirus from patients with pneumonia in China, 2019. N ENGL J MED. 2020; 382(8):727-733. The adaptive immune response to SARS-CoV-2 remains ill-defined and there is an urgent need to fill this gap in knowledge to enable the development of effective vaccines and therapies.

Antibody responses to the spike and nucleocapsid proteins are well described; Long Q X, et al. Antibody responses to SARS-CoV-2 in patients with COVID-19. NAT MED. 2020; 26(6):845-848; Burbelo P D, et al. Detection of nucleocapsid antibody to S4RS-CoV-2 is more sensitive than antibody to Spike protein in COViD-19 patients. J INFECT DIS. 2020; 222:206-213.

Recently the characterization of T-cell responses to SARS-CoV-2 predominantly to spike, membrane, and nucleocapsid proteins has been reported; Grifoni A, et al. Targets of T cell responses to SARS-CoV-2 coronavirus in humans with COVID-19 disease and unexposed individuals. CELL. 2020; 181(7): 1489-1501.

Virus-specific T cells have been used to boost the immunity of immunosuppressed patients, such as those who have undergone allogenic stem cell transplantation; Blyth E, et al. Donor derived CMV-specific T cells reduce the requirement for CMV-directed pharmacotherapy after allogeneic stem cell transplantation. BLOOD. 2013; 121(18):3745-3758. However, methods for inducing and expanding antigen-specific T cells are slow and in many cases too slow to effectively treat a subject who has contracted a viral infection. Moreover many peptide epitopes of SARS-CoV-2 have not been identified or characterized.

In view of the above, the inventors sought to identify peptide epitopes of SARS-CoV-2 and to generate peptide antigen-specific T cells to determinants of this virus as well as identify immunodominant or broadly recognized peptide epitopes. They also sought to design a fast and efficient method for inducing and expanding SARS-CoV-2 specific T cells from convalescent or naïve donors which can be used to treat vulnerable individuals.

### BRIEF SUMMARY OF THE INVENTION

One aspect of the disclosure is directed to a method for preventing or treating SARS-CoV-2 infection by administering SARS-CoV-2-specific T cells (“CSTs”).

A related aspect of the invention is the identification and characterization of peptide epitopes of SARS-CoV-2 spike (S), nucleocapsid (N), membrane proteins, and envelope proteins, especially immunodominant or broadly cross-reactive epitopes, for use in preventing or treating infection by this virus.

Another aspect of the invention involves a method for efficiently generating ex vivo SARS-CoV-2-specific T cells using the peptide epitopes identified and disclosed herein.

Other aspects of the invention pertain to peptide-based or nucleic acid-based vaccines which express these peptide epitopes, methods of treatment using (or expressing) immunogens comprising these epitopes, and to a diagnostic method using the identified peptide epitopes to identify subjects who have SARS-CoV-2 specific T cells in their blood.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings below.

FIG. 1. T-cell recognition of SARS-CoV-2 viral antigens. Specificity of expanded cells in response to SARS-CoV-2 antigens from convalescent patients (n=46) and unexposed controls (n=15) was assayed by IFN-γ ELISpot assay (bars=median). Unstimulated T cells (control [CTL] only) and stimulation with actin were used as negative controls. Results are presented as spot-forming units (SFC) per 1×10⁵ cells. Specificity was defined as ≥20 spots per well with significance above background (actin) via 2-tailed Student t test. Controls (●); Convalescent patients (▾). *P=0.0008, **P=6.24 ×10⁻⁶.

FIGS. 2A-2C. Specificity of ex vivo-expanded SARS-CoV-2-specific T cells (“CSTs”). Following 10 to 12 days of culture, specificities of CD4 and CD8 T-cell populations for membrane, spike, and nucleocapsid proteins were assessed by intracellular cytokine staining for IFNγ and TNF-α. Circles (●). IFN-γ⁺; inverted triangles (▾): TFN⁺; triangles (▴): IFN-γ/TNF⁺.

FIG. 2A. Subject 2 demonstrated a CD4-predominant response targeting structural proteins.

FIG. 2B. Summary data of the response of expanded CD4⁺ T cells in response to membrane, nucleocapsid, and spike proteins by intracellular cytokine staining was analyzed in convalescent donors (n=11). The percentages of T cells were compared with actin-stimulated controls via 2-tailed Student t test. *P<0.05, **P<0.01.

FIG. 2C. Summary data of the response of expanded CD8⁺ T cells in response to membrane, nucleocapsid, and spike proteins by intracellular cytokine staining was analyzed in convalescent donors (n=11). The percentages of T cells were compared with actin-stimulated controls via 2-tailed Student t test. *P<0.05, **P<0.01.

FIG. 3. T-cell specificity of seropositive vs seronegative patients. Comparison of IFN-γ ELISpot results from post expansion CSTs from SARS-CoV-2 seropositive vs. seronegative convalescent patients was performed via Student t test. *P=0.0015, **P=0.00075. Circles (●): controls; squares (▪): seropositive COVID patients; triangles (▴): seronegative COVID patients.

FIG. 4A-4C. SARS-CoV-2 epitope mapping of CSTs. T-cell epitope mapping of structural proteins was performed using minipools containing 8 to 24 peptides each, with responses measured via IFN-γ ELISpot (SFC per 1 ×10⁵ cells).

FIG. 4A shows epitopes within membrane protein that were identified within the C terminus at AA 144-163 and 173-192, which were recognized by 8 and 6 donors, respectively.

FIG. 4B describes mapping of spike protein epitopes demonstrated in three regions at AA 57-75, 205-224, and 449-463, which were recognized by 3 donors.

FIG. 4C describes SARS-CoV-2 mapping of nucleocapsid protein epitopes and showed 2 regions at AA 357-271 and 313-335 that were recognized by 3 donors.

FIGS. 5A-5E. T-cell restrictions of SARS-CoV-2 epitopes. Identification of the T cells responding to each identified epitope was performed via intracellular cytokine staining on expanded CSTs, with percentages of TNF-α⁺/IFN-γ⁺ populations depicted. Intracellular cytokine staining demonstrated a predominant CD4-mediated response to membrane peptides 37-38 (SEQ ID NOS: 53 and 54; FIG. 5A), membrane peptides 44-45 (SEQ ID NOS: 60 and 61; FIG. 5B), nucleocapsid peptide 65 (FIG. 5C), and spike protein peptides 15-16 (FIG. 5D), and a predominant CD8-mediated response to nucleocapsid peptide 81 (FIG. 5E). SEB, staphylococcal enterotoxin β.

FIGS. 6A-6C. Epitope locations within SARS-CoV-2 structural proteins.

FIG. 6A: Epitopes within membrane protein were identified at the C-terminal intravirion domain. TMD, transmembrane domains.

FIG. 6B: Epitopes within spike proteins were found within the S1 region, including one epitope within the receptor-binding domain (RBD).

FIG. 6C: In nucleocapsid protein, epitopes were identified in the region of the dimerization domain (DD).

FIG. 7. Clinical Characteristics of Convalescent COVID-19 Patients. Flow diagram of illness severity (based on WHO classifications), T-cell and antibody immune response to SARS-CoV-2, and basis of COVID-19 diagnosis.

FIG. 8. SARS-CoV-2 Antibody Testing of Normal Controls and Convalescent Patients. Testing for antibodies to Nucleocapsid protein was performed via Luciferase Immunoprecipitation assay. Positivity thresholds (dotted lines) were set based on previous data using unexposed normal control samples.

FIG. 9. Antibody Testing of Normal Controls and Convalescent Patients. Testing for antibodies to Spike protein was performed via Luciferase Immunoprecipitation assay. Positivity thresholds (dotted lines) were set based on previous data using unexposed normal control samples.

FIG. 10 Detection of T-cell responses to SARS CoV-2 proteins from peripheral blood. Peripheral blood mononuclear cells (PBMC) from convalescent patients (triangles) and unexposed controls (circles) were tested for responses to peptide libraries encompassing SARS-CoV-2 structural proteins by IFN-γ ELISpot. Results are reported as spot forming colonies (SFC) per 1×10⁵ cells per well. PBMC alone and actin stimulation were utilized as negative controls. Peptide libraries from cytomegalovirus pp65 and IE1 as well as adenovirus hexon and penton were utilized as additional viral controls.

FIGS. 11A-11C. Extended cell phenotyping of Coronavirus-specific T-cell products.

FIG. 11A. Lymphocyte populations following expansion were determined via flow cytometry.

FIG. 11B. CD4 subpopulation phenotyping of CSTs versus virus-specific T-cells (VSTs) targeting other viruses (CMV, EBV, and adenovirus).

FIG. 11C. Gating strategy for T-cell memory/naive subsets. T-cells were classified as central memory (CD45RO⁺/CCR7⁺/CD95⁺), effector memory (CD45RO⁺/CCR7⁻), and stem cell memory (CD45RO⁻/CCR7⁺/CD95⁺).

FIG. 11D. Gating Strategy for CD4 T-cell subsets. CD4⁺ populations were classified as Th1 (CXCR3+/CXCR5−/CCR6+), Th2 (CCR4⁺/CXCR3⁻/CXCR5⁻), T follicular helper cells (CXCR3⁻/CXCR5+/CCR6⁺), or T-regulatory (CD25high/CD127^{low}).

FIG. 12. SARS-CoV-2-specific T-cell responses via microscale expansion. T-cell responses to SARS-CoV-2 antigens were tested via intracellular flow cytometry following 10 days of expansion in 96-well plates. Data displayed from Subject 16.

FIG. 13. T-cells Responses to Variant COVID-19 Epitopes. Coronavirus-specific T-cells were tested via IFN-γ ELISpot for specificity to peptides corresponding to mutated epitopes in circulating genotypes of SARS-CoV-2, and to homologous nucleocapsid epitopes from NL63 and OC43. First column: SEQ ID NO: 122 (Peptide 53), SEQ ID NO: 490 (variant); SEQ ID NO: 450 (Peptide 65), SEQ ID NO: 491 (variant); SEQ ID NO: 60 (Peptide 44), and SEQ ID NO: 492 (variant). Second column showing coronavirus OC43 and NL63 peptides: SEQ ID NOS: 450 (Peptide 65), SEQ ID NO: 488, SEQ ID NO: 450 (Peptide 65), SEQ ID NO: 489.

FIG. 14. Correlation of Spike antibody and T-cell responses. Log-transformed spike responses (antibody and IFN-γ ELISpot results) were evaluated via Q-Q plots. Correlation between the biomarkers using Pearson's product moment correlation coefficient yielded a value of 0.423, with p=0.004.

FIG. 15. T-cell Responses to SARS-CoV-2 versus Illness Severity in Convalescent Patients. Expanded coronavirus-specific T-cells (culture day 10) were tested for specificity to SARS-CoV-2 structural protein libraries via IFN-γ ELISpot. Control unexposed donors (black circles) and convalescent patients with mild disease (triangles) or moderate to severe disease (red triangles) by WHO criteria were tested. Expanded cells alone (CTL alone) and actin stimulated cells were used as negative controls. SFC: spot forming colonies.

FIG. 16. SARS-CoV-2 specific T-cell responses in subjects before and after COVID-19 recovery. T-cell responses against SARS-CoV-2 viral proteins was evaluated in Subject 4 and Subject 46 from samples banked before the COVID-19 pandemic, as well as from samples obtained after the subjects' recovery from COVID-19. Cells were expanded for 10 days, followed by testing via IFNγ ELISpot. SFC: spot forming colonies.

FIG. 17. HLA Restriction Testing of SARS-CoV-2 specific T-cell clones. T-cell clones from Subject 6 were tested with HLA blocking antibodies targeting HLA-DR, HLA-DQ, or pan-class II, and stimulated with Spike or Membrane peptide pools, followed by intracellular flow cytometry.

FIG. 18. HLA Restriction Mapping of Membrane Peptide 37. HLA restriction of Membrane peptide 37 was determined by testing of CSTs with peptide-pulsed, partially-HLA matched PHA blasts via IFN-γ ELISpot assay.

FIG. 19. Demonstration that SARS-CoV-2-specific T cells can be prepared from cord blood (CB).

FIG. 20. Convalescent donors recognize multiple SARS-CoV-2 structural proteins, especially spike (S), Nucleocapsid (N) and membrane protein (inverted triangles) compared to normal controls (circles).

FIG. 21. Convalescent patients (Subject 46) mount new T cell and antibody responses to SARS-CoV-2 antigens. Top panel in order from top, responses to spike, membrane, nucleoprotein, and membrane protein at bottom. Bottom panel nucleocapsid top line, spike, bottom line.

FIG. 22. Description of a method for generating coronavirus-specific T cells using IL-4 and IL-7.

FIG. 23. Seropositive donors recognize a broader range of SARS-CoV-2 proteins. Controls (circles), seropositive patients (squares), seronegative patients (triangles).

FIG. 24. Description of donor-derived and third-party T cell therapies for COVID-19.

### DETAILED DESCRIPTION OF THE INVENTION

As shown herein, the inventors identified peptide epitopes of SARS-CoV-2 important for priming and expanding SARS-CoV-2-specific T cells and useful for prevention and treatment of SARS-CoV-2 infections like COVID-19. They identified a set of immunodominant T-cell epitopes within conserved regions of SARS-CoV-2 structural proteins and observed SARS-CoV-2-specific T cells predominantly recognize regions in the C-terminus of the SARS-CoV-2 membrane protein, thus revealing a critical “hot spot” for CD4-restricted T-cell epitopes. An association between SARS-CoV-2 seropositivity and the breadth of T cell responses to structural viral proteins in patients who recover from COVID-19 was also identified. These findings are considered to indicate that patients who mount an antibody response to SARS-CoV-2 are more likely to have a broader T-cell response following exposure to SARS-CoV-2.

These findings provide a basis for selection of PBMC donors based on antibody levels to SARS-CoV-2 antigens. The use of PBMCs from such donors can accelerate the production of antigen-specific T cells to SARS-CoV-2 as well as provide a broader cellular immunological response to a greater number of SARS-CoV-2 antigens or to variant antigens from mutated SARS-CoV-2 strains.

Antigen-specific T cells produced from PBMCs of such donors offer rapid and attractive way to treat patients most at risk of SARS-CoV-2 infection including immunosuppressed patients or patients who have undergone bone marrow transplantation (BMT).

Embodiments of this disclosure include, but are not limited to the following.

One aspect of this technology is directed to a method for treating a subject infected with, or at risk of infection by, a coronavirus, such as SARS-COV-2 using T cells that recognize peptide antigens or epitopes of SARS-CoV-2.

The method comprises administering to a subject in need thereof ex vivo primed and/or expanded SARS-CoV-2 antigen-specific T cells that recognize at least one peptide antigen or epitope consisting of Peptide 37 (SEQ ID NO: 53), Peptide 38 (SEQ ID NO: 54), Peptide 44 (SEQ ID NO: 60), Peptide 45 (SEQ ID NO: 61) or any one of SEQ ID NOS: 1-524 or a class 1 or class 2 restrictable fragment thereof. In some embodiments, the T cells may recognize conserved or cross-reactive peptides from other coronaviruses or be primed and expanded using such conserved or cross-reactive peptides. In some embodiments a donor from whom ex vivo primed or expanded T cells are derived may be seropositive to one or more SARS-CoV-2 antigens, such as a convalescent SARS-CoV-2 patient or a subject who has been immunized against SARS-CoV-2. In other embodiments, the donor may be seronegative to one or more SARS-CoV-2 antigens or a subject who has not been vaccinated against SARS-CoV-2.

As used herein the terms “peptide antigen” and “peptide” can refer to the same structural molecule and can be used synonymously. Such a molecule may be antigenic or immunogenic in some individuals depending on their immunological background or ability to restrict a peptide antigen or epitope via MHC.

In some embodiments, the T cells may already be primed by exposure to SARS-CoV-2 or to other coronavirus antigens and the method is used to expand their numbers or further refine their ability to recognize SARS-CoV-2 peptides when restricted by a major histocompatibility antigen (or HLA) or their other functional or phenotypic properties.

In one embodiment, the ex vivo primed or expanded SARS-CoV-2 antigen-specific cells are derived from PBMCs or other hematopoietic cells taken from a donor previously exposed to SARS-CoV-2 whose antibody levels to one or more SARS-CoV-2 antigens are greater than a control value from an uninfected or unvaccinated subject or from a group of subjects. Further priming or expansion of cells from a non-naïve donor can accelerate the production of SARS-CoV-2-specific T cells or focus T cell responses on immunodominant determinants or epitopes.

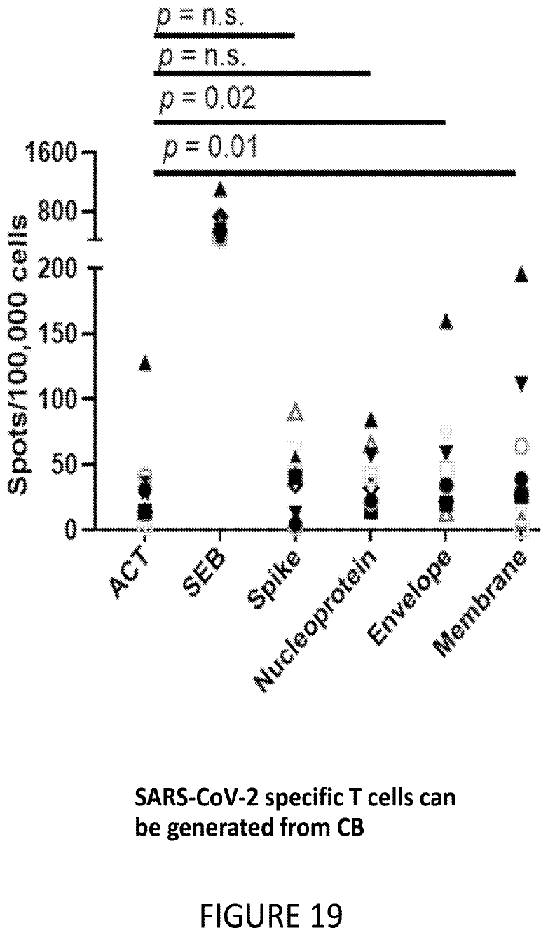

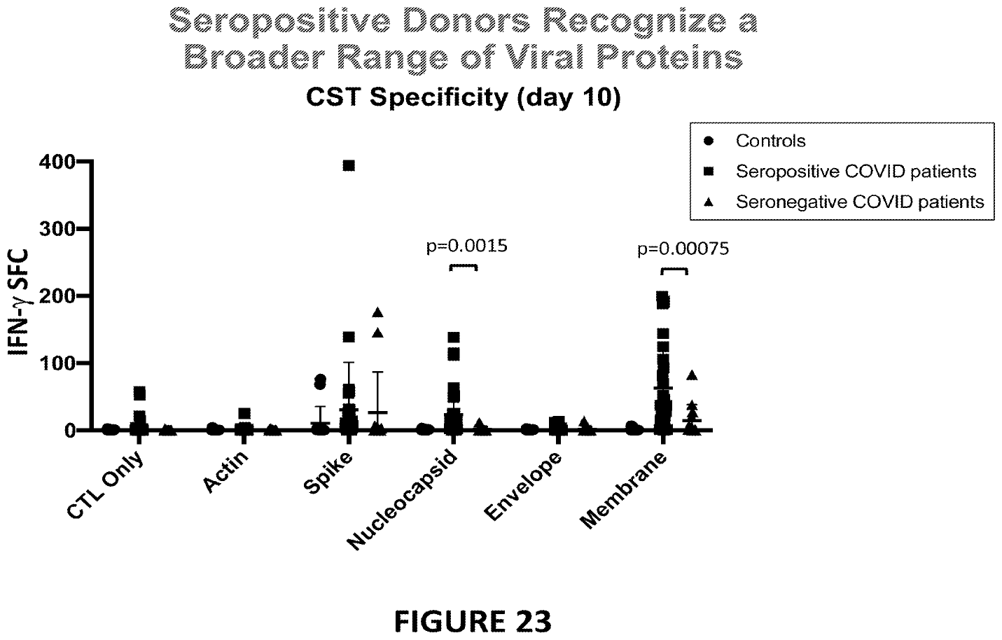

In another embodiment, the ex vivo primed or expanded SARS-CoV-2 antigen-specific cells are derived from PBMCs or other hematopoietic cells taken from a donor whose antibody levels to one or more SARS-CoV-2 antigens are no more than a control value from an uninfected or unvaccinated subject. In some instances, naïve PBMCs or PBMCs lacking memory cells to SARS-CoV-2 antigens may be used, including, but not limited to, cord blood cells. FIG. 19 demonstrates that SARS-CoV-2 specific T cells are generated from cord blood. Further priming or expansion of cells from a naïve (to SARs-CoV-2 epitopes) donor can result in production of T cells not biased by prior cellular responses to SARS-CoV-2 antigens. FIG. 23 shows that T cells derived from seropositive donors recognize a broader range of viral proteins than T cells derived from seronegative donors.

In some instances, the control value may be zero or close to zero indicating that the control subject or control population has few or no antibodies that recognize SARS-CoV-2 antigens. In other instances, the control value may show the presence of some antibodies that recognize certain SARS-CoV-2 antigens, but at levels which are 95, 90, 80, 70, 60, 50, 40, 30, 20, 10, 5, or 1% of those found to the same SARS-CoV-2 antigen or epitope in a subject or population that has been previously infected by SARS-CoV-2 or immunized against SARS-CoV-2.

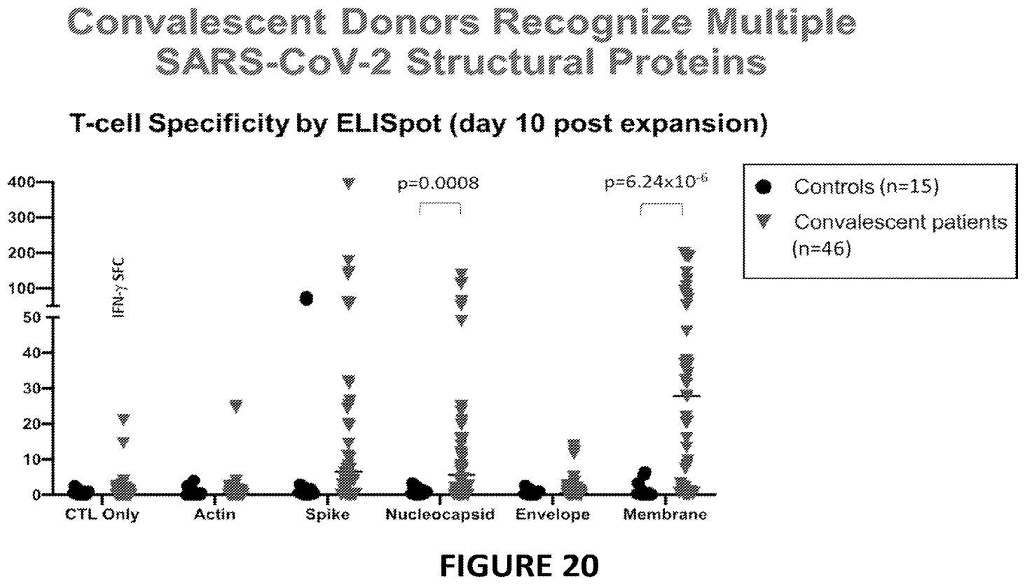

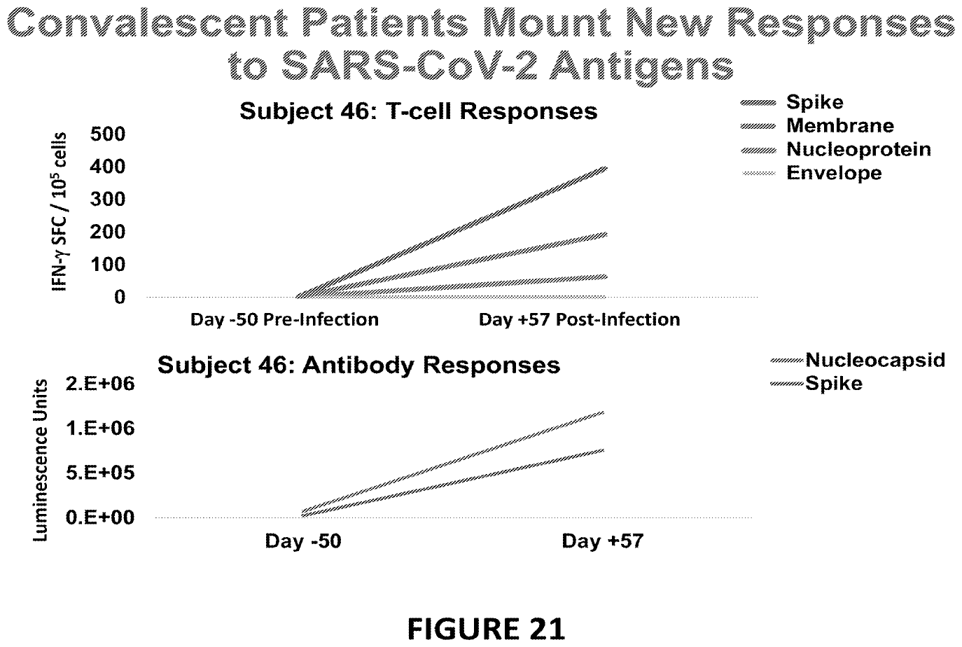

In one embodiment, the T cells are derived from a donor who is convalescing or has convalesced from SARS-CoV-2 infection. As shown by FIG. 20, T cells expanded from convalescent donors can recognize multiple SARS-CoV-2 structural proteins. FIG. 21 shows that convalescent SARS-CoV-2 patients can mount new cellular responses to SARS-CoV-2 antigens.

In another embodiment, the T cells are derived from a donor who has been immunized to at least one SARS-CoV-2 antigen.

In another embodiment, the T cells are derived from donor who has not been previously exposed to SARS-CoV-2 or said donated cells are naïve to one or more SARS-CoV-2 antigens. Such cells may include naïve stem cells or cord blood cells.

In some embodiments, the peptide antigen is MHC or HLA class 1 restricted. For example, the peptide antigen may be restricted or capable of being restricted by an HLA-A, HLA-B or HLA-C antigen of a donor. In preferred embodiments the class 1 restricted peptide antigen ranges in length from 8 to 25, preferably, from 8, 9 to 10 amino acid residues.

In some embodiments, the peptide antigen is MHC- or HLA-class 2 restricted. For example, the peptide antigen may be restricted or capable of being restricted by an HLA-DRB1, HLA-DRB3, HLA-DRB4, HLA-DRB-5, HLA-DQA1, HLA-DQB1, HLA-DPA1, or HLA-DPB1 antigen of a donor. In preferred embodiments the class 1 restricted peptide antigen ranges in length from 8 to 25, preferably, from 13, 14, 15, 16, to 17 amino acid residues.

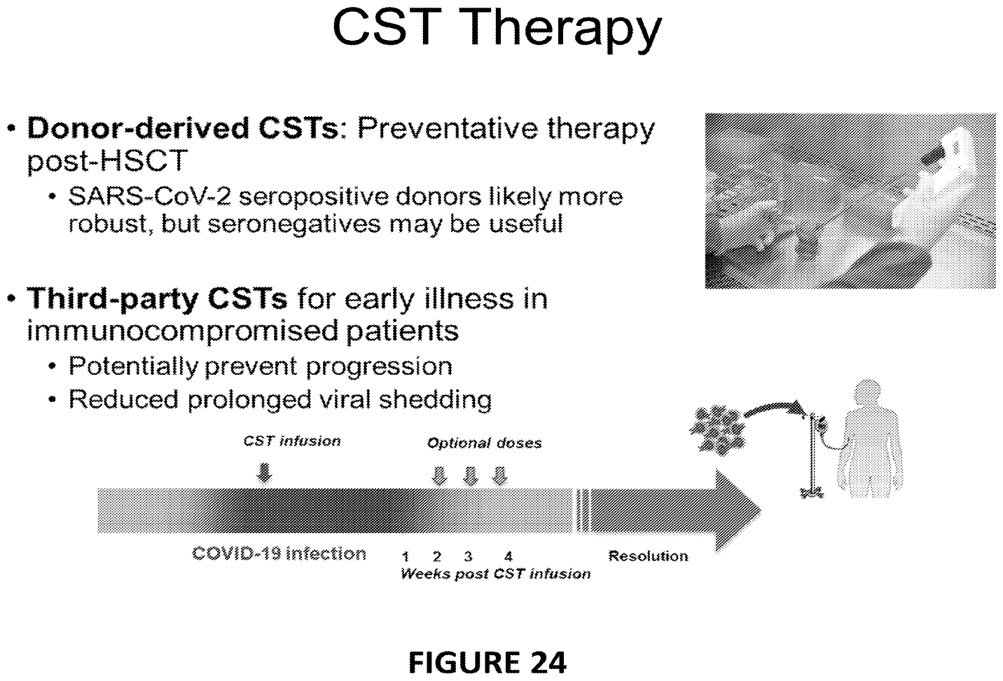

As described by FIG. 24, the SARS-CoV-2 specific T cells may be donor-derived, preferably from a SARS-CoV-2 seropositive donor, and used for preventative therapy, for example, after HSCT (hematopoietic stem cell transplantation). Third party SARS-CoV-2-specific T cells (“CSTs”), such as cryogenically preserved, patient-matched CSTs, may be used to prevent early illness associated with SARS-CoV-2 infection and prevent disease progression. Typically, a third party donor must meet stringent suitability and eligibility requirements.

In one embodiment the peptide antigen comprises a segment of SARS-CoV-2 membrane protein, such as one of the following peptides: Leu Arg Gly His Leu Arg Ile Ala Gly His His Leu Gly Arg Cys (SEQ ID NO: 53), Leu Arg Ile Ala Gly His His Leu Gly Arg Cys Asp Ile Lys Asp (SEQ ID NO: 54), Ser Arg Thr Leu Ser Tyr Tyr Lys Leu Gly Ala Ser Gin Arg Val (SEQ ID NO: 60), Ser Tyr Tyr Lys Leu Gly Ala Ser Gln Arg Val Ala Gly Asp Ser (SEQ ID NO: 61), or Leu Gly Ala Ser Gin Arg Val Ala Gly Asp Ser Gly Phe Ala Ala (SEQ ID NO: 62).

In another embodiment, the peptide antigen comprises a segment of SARS-CoV-2 nucleocapsid protein, such as one of the following peptides: Lys Pro Arg Gln Lys Arg Thr Ala Thr Lys Ala Tyr Asn Val Thr (SEQ ID NO: 450), Ala Phe Phe Gly Met Ser Arg lie Gly Met Glu Val Thr Pro Ser (SEQ ID NO: 464); Met Ser Arg Ile Gly Met Glu Val Thr Pro Ser Gly Thr Trp Leu (SEQ ID NO: 465) or Gly Met Glu Val Thr Pro Ser Gly Thr Trp Leu Thr Tyr Thr Gly (SEQ ID NO: 466).

In another embodiment, the peptide antigen comprises a segment of SARS-CoV-2 spike (S) protein, such as: Pro Phe Phe Ser Asn Val Thr Trp Phe His Ala Ile His Val Ser (SEQ ID NO: 84), Asn Val Thr Trp Phe His Ala Ile His Val Ser Gly Thr Asn Gly (SEQ ID NO: 85), Ser Lys His Thr Pro Ile Asn Leu Val Arg Asp Leu Pro Gln Gly (SEQ ID NO: 121); Pro Ile Asn Leu Val Arg Asp Leu Pro Gin Gly Phe Ser Ala Leu (SEQ ID NO: 122), or Tyr Asn Tyr Leu Tyr Arg Leu Phe Arg Lys Ser Asn Leu Lys Pro (SEQ ID NO: 182).

In one embodiment, the peptide used to prime, expand, or prime and expand donor T cells is derived from the envelope protein.

In some embodiments, the peptide or peptide antigen used to prime and/or expand T cells is an antigen or other than the S, N, membrane or envelope antigens.

In one embodiment, the SARS-CoV-2 antigen-specific T cells are autologous or fully histocompatible to the subject.

In another embodiment, the SARS-CoV-2 antigen-specific T cells are allogenic, partially histocompatible, not histocompatible, xenogeneic, recombinant, or artificial to the subject.

In another embodiment the SARS-CoV-2 antigen-specific T cells are non-autologous and share at least 1, 2, 3, 4, 5, 6 or more major histocompatibility antigens with the subject.

In another embodiment, the SARS-CoV-2 specific T cells are administered to a subject at least <1, 1, 2, 3, 4, 5, 6, 7, 8, 9, 10, 15, 18, 21, 30, 35, 40, 45, 50, 55, 60, 65, 70, 75, 80, 85, 90, 95, 100, or >100 years old, especially to elderly subjects.

Other treatable subjects or patient populations which may be treated by the methods disclosed herein, including by not limited to adoptive transfer of T cells, include smokers, drug abusers, those with COPD or respiratory diseases or conditions, asthmatics, diabetics, those with high blood pressure, heart disease or coronary artery disease, cancer, those who have undergone bone marrow or stem cell transplantation or transplantation of other tissue, and subjects who are immune suppressed. Treatable subjects also include those who have undergone radiotherapy or chemotherapy for cancer or other diseases; or who are immunosuppressed, immuno-incompetent, immunodeficient, or immunocompromised, such as a subject who has undergone BMT.

A stem cell or bone marrow transplant (BMT) replaces damaged blood cells with healthy ones, but can erase previously acquired cellular immunity. BMT is often used to treat conditions affecting the blood cells, such as leukemia and lymphoma. Stem cells are special cells produced by bone marrow (a spongy tissue found in the centers of some bones) that can turn into different types of blood cells. The three main types of blood cells they can become are red blood cells-which carry oxygen around the body, white blood cells-which help fight infection, platelets-which help stop bleeding. A stem cell transplant involves destroying any unhealthy blood cells and replacing them with stem cells removed from the blood or bone marrow. A bone marrow transplant can regenerate a new immune system that will fight existing or residual leukemia or other cancers not killed by the chemotherapy or radiation used in the transplant; replace the bone marrow and restore its normal function after high doses of chemotherapy and/or radiation are given to treat a malignancy. This process is often called rescue; or replace bone marrow with genetically healthy functioning bone marrow to prevent more damage from a genetic disease process (such as Hurler's syndrome and adrenoleukodystrophy). The T cell-based treatments disclosed herein may be used with those who have undergone BMT or prior to a future BMT including but not limited to those with leukemia, severe aplastic anemia, lymphomas, multiple myeloma, immune deficiency disorders, and some solid-tumor cancers.

In another embodiment, the subject is a doctor, nurse, healthcare worker, emergency medical technician, or member of the fire department, police, or military, transportation or delivery, or is necessary for continuity of government, and others at higher risk of infection by SARS-CoV-2.

In one embodiment, the SARS-CoV-2 specific T cells are administered parenterally, for example, by intravenous infusion, intraperitoneal infusion, or other parenteral mode. T cells may also be infused or administered to a site of SARS-CoV-2 infection such as into the lungs or upper or lower respiratory system or into or around another infected tissue or organ.

Another aspect of this technology is a method for selecting a PBMC or hematopoietic cell donor for ex vivo priming or expansion of SARS-CoV-2-specific T cells comprising:

detecting antibodies that recognize SARS-CoV-2 antigens in a biological sample from a subject previously infected with SARS-CoV-2,

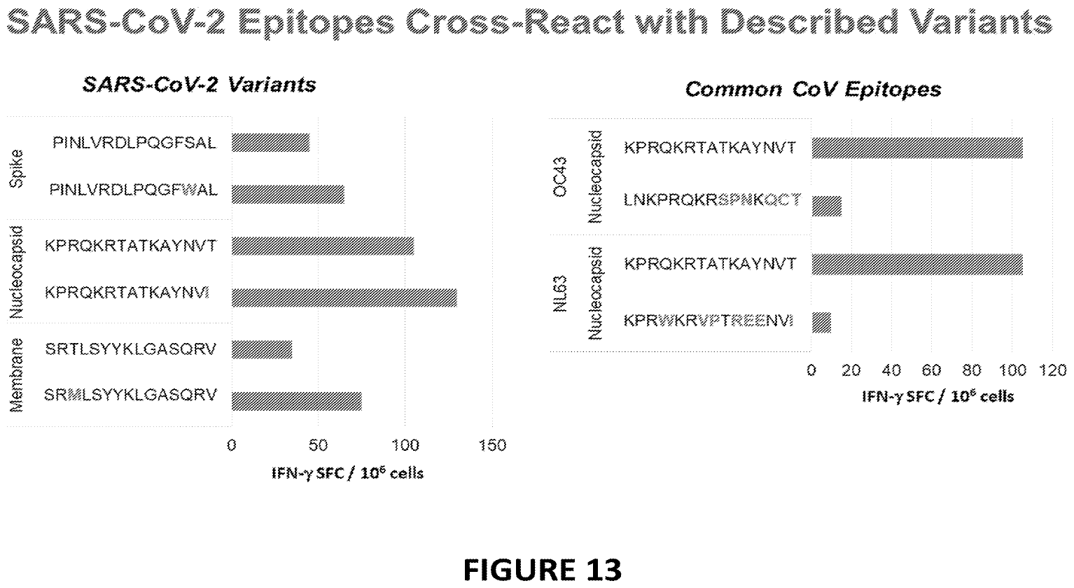

obtaining peripheral blood mononuclear cells (PBMCs) or hematopoietic cells from the subject when the levels of SARS-CoV-2 antibodies detected exceed those in a previously uninfected subject or a normal control value, or when a greater number SARS-CoV-2 antigens are detected in the blood of the previously infected subject than in a previously uninfected subject or normal control value, and priming or expanding SARS-CoV-2-specific T cells in the PMBCs by exposing the PBMCs to one or more of the peptide antigens described by SEQ ID NOS: 1-524 or peptides antigens having conserved or cross-reacting epitopes therewith. As shown by FIG. 13, T cells can cross-react with epitopes of SARS-CoV-2 variant strains and in some cases common CoV epitopes. These CSTs were generated via a 10-day expansion targeting the SARS-CoV-2 reference sequences antigens (Wuhan Hu-1) with IL-4/IL-7 and then tested via IFN-γ ELISpot against the variant peptides corresponding to Spike, Membrane, and Nucleocapsid epitopes as listed.

In some embodiments, a convalescent subject may have recovered from SARS-CoV-2 infection <1, 1, 2, 3, 4, 5, 6, 12, 18, 24 or >24 months prior to use of their PBMCs or hematopoietic cells as donor cells. Similarly, an immunized subject may have been immunized against SARS-CoV-2<1, 1, 2, 3, 4, 5, 6, 12, 18, 24 or >24 months prior to use of their PBMCs as donor cells. In other embodiments, an immunized subject and potential PBMC donor may have received 1, 2 or more doses of a SARS-CoV-2 protein- or peptide-based or nucleic acid-based vaccine. In some cases, SARS-CoV-2 neutralizing antibodies are detected and used to select a suitable donor.

In one embodiment, the antigens used to detect antibodies to SARS-CoV-2 are the S, N, membrane or envelope antigens or fragments thereof comprising at least one epitope recognized by an antibody. Measurements may comprise determining levels of antibodies to only S, only N, only membrane, or only envelope protein or epitopes from these proteins. In other embodiments, antibodies binding to S and N, S and membrane, S and envelope protein may be detected, compared to a control value, and used to select a donor. In another embodiment, antibodies to N and membrane or N and envelope protein may be measured, compared to a control value, and used to select a donor. Antibody levels to all SARS-CoV-2 antigens may also be detected and used to select a donor.

In another embodiment, the antigen which is recognized by antibodies may consist or comprise at least one peptide antigen of SEQ ID NO: 1-524.

In one embodiment of this method, the PBMCs or hematopoietic cells are obtained when the biological sample has a higher level of antibodies to N protein, S protein, membrane protein and/or envelope protein or higher levels to SARS-CoV-2 antigens in general, than the control sample or a standardized control value, such as that from a population of subjects of the same age, sex, or medical condition (e.g., obese, diabetic, cardiovascular disease, immunosuppressed, post-transplant patient, etc.). Controls may comprise uninfected and/or unvaccinated subjects whose immune systems have not been exposed to SARS-CoV-2 antigens or nucleic acids. In some embodiments, such unexposed controls may be further selected based on other factors such as age, gender, genetic background or race, or medical history or condition. Similarly, positive controls may be selected from those previously infected or vaccinated with SARS-CoV-2 antigens or nucleic acids and also preferably matched by the factors described. Control values may be taken from one or more subjects or from a population of subjects.

In other embodiments, higher levels of antibodies recognizing SARS-CoV-2 antigens, such as S, N, membrane, and envelope antigens, or peptide epitopes derived therefrom may be used. The presence or level of one, two, three of more antibody isotypes, such as IgA, IgD, IgE, IgG, or IgM, which recognize SARS-CoV-2 antigens or their peptide epitopes may be measured and donors selected based on relative levels of such antibodies compared to uninfected subjects or a control value from a population of uninfected subjects.

Detection/Quantitation of Anti-SARS-CoV-2 Antibodies in a Biological Sample. As described above, the level of anti-SARs-CoV-2 antibodies in a subject (e.g., a human or a non-human mammal susceptible to infection) helps identify donors whose PBMCs can rapidly and efficiently be expanded to produce SARS-CoV-2-specific T cells.

Assay methods to detect anti-SARS-CoV-2 antibodies in a sample from a subject can be carried out in any of a wide variety of formats. In certain assays, antibodies to SARS-CoV-2 antigens or peptide epitopes can be assessed using immunoassays. Immunoassay formats are preferred, e.g., those selected from the group consisting of, an immunoblot, a Western blot, a dot blot, an enzyme linked immunosorbent assay (ELISA), radioimmunoassay (RIA), enzyme immunoassay. Modified immunoassays utilizing fluorescence resonance energy transfer (FRET), biosensor technology, evanescent fiber-optics technology, protein chip technology, and the like are also useful. Preferably, the assay is a semi-quantitative assay or quantitative assay.

Examples of suitable immunoassays are described below and will, in view of the teachings provided herein, be apparent to those skilled in the art. For a general review of immunoassays, see METHODS IN CELL BIOLOGY Volume 37: Antibodies in Cell Biology, Asai, ed. Academic Press, Inc. New York (1993); BASIC AND CLINICAL IMMUNOLOGY 7th Edition, Stites & Terr, eds. (1991), each of which is incorporated by reference in its entirety.

In various embodiments the assays can involve assaying for all antibodies with reactivity to SARS-CoV-2 antigens or peptide epitopes, or only for antibodies of a particular isotype, such as IgM, IgG, IgA, IgD or IgE or for a combination of two or more antibody isotypes. In certain embodiments at least the level of IgG and/or IgM is determined.

Immunoassays can be competitive or noncompetitive. In a typical competitive immunoassay, the antibody in the sample competes with labeled antibody to bind with the SARS-CoV-2 antigen or peptide epitope. The amount of labeled antibody bound to the antigen or peptide epitope is then measured. There is an inverse relationship between concentrations of endogenous anti-SARS-CoV-2 antibody in the sample and the quantity of labeled antibody detected.

In noncompetitive immunoassays, antibody in the sample is bound to the antigen or epitope, then a labeled detection reagent, typically an anti-immunoglobulin antibody, is bound to the antibody. The amount of labeled detection reagent bound to the antibody is then measured. Unlike the competitive method, the results of the noncompetitive method will be directly proportional to the concentration of the antibody.

In a noncompetitive immunoassay or western blot, a labeled detection reagent, typically an anti-immunoglobulin antibody, is used to detect antibody (e.g., anti-SARs-CoV-2 antibody) bound to the SARS-CoV-2 antigen or epitope. A suitable anti-immunoglobulin antibody is chosen that binds specifically to immunoglobulin of the species from which the sample is obtained. In certain embodiments it may bind to all immunoglobulin isotypes of that species, or only a subset of isotypes. For example, it may bind only to IgA, IgD, IgE, IgG or IgM, or combinations of two or more of these isotypes. In certain embodiments the anti-immunoglobulin antibody may bind specifically only to certain subtypes of any given isotype. Subtypes of human IgA include IgA1 and IgA2. In certain embodiments the anti-immunoglobulin antibody may bind to one or both of these subtypes. Subtypes of human IgG include IgG1, IgG2, IgG3 and IgG4. In certain embodiments the anti-immunoglobulin may bind to one or more of these human IgG subtypes. It will be appreciated that there are different isotypes and subtypes in different vertebrate species.

In radioimmunoassay, the antibody or detection reagent is labeled with a radioisotope, such as ¹³¹I or ¹²⁵I. In enzyme immunoassays, the antibody or detection reagent is labeled with an enzyme. In certain embodiments suitable enzymes are capable of being detected with the use of a chromogenic substrate. A chromogenic substrate is a substance which, as a result of the reaction with the enzyme, gives rise to a colored product which can thus be detected spectrophotometrically. Enzymes such as horse radish peroxidase, alkaline phosphatase, beta-galactosidase, and pyrophosphatase from E. coli have been widely employed. Chemi-luminescent systems based on enzymes such as luciferase can also be used. Other labels include fluorescent labels such as fluorophores of the Alexa series, quantum dots, electron spin labels, magnetic labels, and the like. In certain embodiments conjugation of the antibody or detection reagent with the biotin is frequently used since this can readily be detected by its reaction with enzyme- or fluorophore-linked avidin or streptavidin to which it binds with great specificity and affinity. Alternatively, in certain embodiments, the antibody/detection reagent is conjugated with streptavidin or avidin that binds a detection reagent linked biotin.

In one illustrative and typical noncompetitive enzyme immunoassay, the biological sample to be analyzed (e.g., serum) is placed in contact and incubated with a SARS-CoV-2 antigen adsorbed on (or chemically linked to) a solid (or substantially solid) substrate. Any SARS-CoV-2 antibodies that are possibly present in the sample are thus specifically bound by the SARS-CoV-2 antigen attached to the solid substrate, producing SARS-CoV-2 antigen/anti-SARS-CoV-2 antibody complex. The sample is then separated from the solid substrate so as to eliminate non-bound materials, for example, by washing. An indicator antibody capable of binding anti-SARS-CoV-2 antibodies that are present on the substrate in the form of a SARS-CoV-2 antigen/anti-SARS-CoV-2 antibody complex is added to the solid substrate, thus producing a SARS-CoV-2 antigen/anti-SARS-CoV-2 antibody/indicator antibody complex. The indicator antibody may, for example, be an anti-human IgG immunoglobulin (or anti-human IgM immunoglobulin, or anti-human IgA immunoglobulin, etc.) raised in a non-human animal species. Finally, the presence of the SARS-CoV-2 antigen/anti-SARS-CoV-2 antibody/indicator antibody complex on the solid substrate is detected and/or quantified, the presence of said complex on the solid substrate being indicative of the presence of anti-SARS-CoV-2 antigen antibodies in the sample and the amount of the complex being indicative of the amount of anti-SARS-CoV-2 antigen antibodies in the sample.

In certain embodiments it is preferred that a quantitative estimate of antibody that can bind to the SARS-CoV-2 antigen or peptide epitope is obtained. In typical non-competitive assays, a linear relationship between the measured variable, whether it be optical density or some other read-out, and antibody concentration, is assumed. For example, if sample A has double the optical density of sample B in the assay (background having been subtracted from both), it is assumed that the concentration of antibody is double in A compared to B. However, it is preferable to construct a standard curve of serial dilutions of a pool of positive samples (e.g., serum samples). In certain embodiments such dilutions are assayed at the same time as the test samples. By doing this, any variation from the linear relationship may be taken into account in determining the quantity of antibody in the samples.

In some embodiments, the level of antibodies to SARS-CoV-2 antigen or epitope in a biological sample will be at least 1, 2, 5, 10, 25, 50, 100, 125, 150 or 200% (or any intervening value or subrange) more than those in a control sample, such as a sample from an uninfected donor or from the same donor prior to infection with SARS-CoV-2.

In certain embodiments the solid substrate is a micro-titration plate, for example, of the type commonly used for performing ELISA immunological assays. In certain embodiments the micro-titration plate is preferably a polystyrene plate. Useful solid supports also include, but are not limited to natural polymeric carbohydrates and their synthetically modified, crosslinked, or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer. All of these materials may be used in suitable shapes, such as films, sheets, tubes, particulates, or plates, or they may be coated onto, bonded, or laminated to appropriate inert carriers, such as paper, glass, plastic films, fabrics, or the like.

Illustrative solid phase materials well suited for flow-through assay devices include, but are not limited to filter paper such as a porous fiberglass material or other fiber matrix materials. The thickness of such material is not critical and will be a matter of choice, largely based upon the properties of the sample or analyte being assayed, such as the fluidity of the biological sample.

In certain embodiments the solid phase can constitute microparticles (or nanoparticles). Suitable microparticles useful in the methods described herein can be selected by one skilled in the art from any suitable type of particulate material and include, but are not limited to those composed of polystyrene, polymethylacrylate, polypropylene, latex, polytetrafluoroethylene, polyacrylonitrile, polycarbonate, or similar materials. Further, the microparticles can be magnetic or paramagnetic microparticles, so as to facilitate manipulation of the microparticle within a magnetic field.

Microparticles can be suspended in the mixture of soluble reagents and biological sample or can be retained and immobilized by a support material. In the latter case, the microparticles on or in the support material are typically or preferably not capable of substantial movement to positions elsewhere within the support material. Alternatively, the microparticles can be separated from suspension in the mixture of soluble reagents and biological sample by sedimentation or centrifugation. When the microparticles are magnetic or paramagnetic the microparticles can be separated from suspension in the mixture of soluble reagents and biological sample by a magnetic field.

The methods as disclosed herein can be adapted for use in systems that utilize microparticle technology including automated and semi-automated systems wherein the solid phase comprises a microparticle. Such systems include those described in published EPO App. Nos. EP 0 425 633 and EP 0 424 634, respectively, and U.S. Pat. No. 5,006,309.

Thus, for example, it will be appreciated that the illustrative assay described above, can also be performed in a fluid phase. The SARS-CoV-2 antigen can be provided attached to microparticles or nanoparticles that are contacted with the sample in a suspension. Anti-SARS-CoV-2 antigen antibodies present in the sample bind to the SARS-CoV-2 antigen on the microparticles forming a SARS-CoV-2 antigen/anti-SARS-CoV-2 antigen antibody complex on the surface of the microparticles. This complex is then contacted with an indicator antibody capable of binding anti-SARS-CoV-2 antigen antibodies that are present in the SARS-CoV-2 antigen/anti-SARS-CoV-2 antigen antibody complex thus producing a SARS-CoV-2 antigen/anti-SARS-CoV-2 antibody/indicator antibody complex attached to the microparticles. The microparticles can then be separated and the label detected/quantified using for example a cell sorter, or a magnetic separation system.

In certain embodiments, the solid substrate can comprise one or more electrodes. The SARS-CoV-2 antigen or peptide epitope (capture agent) can be affixed, directly or indirectly, to the electrode(s). In one embodiment, for example, the SARS-CoV-2 antigen can be affixed to magnetic or paramagnetic microparticles, which are then positioned in the vicinity of the electrode surface using a magnet. Systems in which one or more electrodes serve as the solid phase are useful where detection is based on electrochemical interactions. Illustrative systems of this type are described, for example, in U.S. Pat. No. 6,887,714. The basic method is described further below with respect to electrochemical detection.

As indicated above, in various embodiments, the SARS-CoV-2 antigen or peptide epitope can be attached to the solid support (e.g. ELSA well, microparticle, test strip, etc.) by any of a number of methods. The attachment can be simple adsorption, ionic bonding, or covalent coupling (directly or through a linker). In one illustrative embodiment, the SARS-CoV-2 antigen or peptide epitope is adsorbed to the solid substrate by incubating the SARS-CoV-2 antigen or peptide epitope in a buffer with the solid substrate. Suitable buffers include, but are not limited to carbonate buffer or phosphate buffered saline. Typically, after adsorption or covalent linkage of the SARS-CoV-2 antigen or peptide epitope to the solid substrate, the solid substrate is incubated with a blocking agent to reduce non-specific binding of matter from the sample to the solid substrate. Suitable blocking agents include, but are not limited to bovine serum albumin.

In certain embodiments the intrinsic charge of the solid substrate is altered to facilitate attachment of the SARS-CoV-2 antigen or peptide epitope, and/or to improve antibody binding, and/or to improve wettability, and the like. In certain embodiments to change or enhance the intrinsic charge of the solid substrate, a charged substance can be coated directly onto the substrate. For example, ion capture procedures for immobilizing an immobilizable reaction complex with a negatively charged polymer, described in EP Patent Publication No. 0326100, and in EP Publication No. 0406473, can be employed to affect a fast solution-phase immunochemical reaction. In these procedures, an immobilizable immune complex is separated from the rest of the reaction mixture by ionic interactions between the negatively charged polyanion/immune complex and the previously treated, positively charged matrix and detected by using any of a number of signal-generating systems, including, e.g., chemiluminescent systems, as described in EPO Publication No. 0 273,115.

If the solid substrate is silicon or glass, the surface is often activated prior to attaching the capture agent (e.g., the SARS-CoV-2 antigen or peptide epitope). Activated silane compounds such as triethoxy amino propyl silane (available from Sigma Chemical Co., St. Louis, Mo.), triethoxy vinyl silane (Aldrich Chemical Co., Milwaukee, Wis.), and (3-mercapto-propyl)-trimethoxy silane (Sigma Chemical Co., St. Louis, Mo.) can be used to introduce reactive groups such as amino-, vinyl, and thiol, respectively. Such activated surfaces can be used to link the capture directly (in the cases of amino or thiol), or the activated surface can be further reacted with linkers such as glutaraldehyde, bis (succinimidyl) suberate, SPPD 9 succinimidyl 3-[2-pyridyldithio] propionate), SMCC (succinimidyl-4-[Nmaleimidomethyl] cyclohexane-1-carboxylate), SIAB (succinimidyl [4iodoacetyl] aminobenzoate), and SMPB (succinimidyl 4-[1maleimidophenyl] butyrate) to separate the capture agent from the surface. Vinyl groups can be oxidized to provide a means for covalent attachment. Vinyl groups can also be used as an anchor for the polymerization of various polymers such as poly-acrylic acid, which can provide multiple attachment points for specific capture agents. Amino groups can be reacted with oxidized dextrans of various molecular weights to provide hydrophilic linkers of different size and capacity. Examples of oxidizable dextrans include Dextran T-40 (molecular weight 40,000 daltons), Dextran T-110 (molecular weight 110,000 daltons), Dextran T-500 (molecular weight 500,000 daltons), Dextran T-2M (molecular weight 2,000,000 daltons) (all of which are available from Pharmacia, Piscataway, N.J.), or Ficoll (molecular weight 70,000 daltons; available from Sigma Chemical Co., St. Louis, Mo.). Additionally, polyelectrolyte interactions can be used to immobilize a SARS-CoV-2 antigen or peptide epitope on a solid phase using techniques and chemistries described U.S. Pat. Nos. 5,459,080, 5,459,078, and the like.

Other considerations affecting the choice of solid phase include the ability to minimize non-specific binding of labeled entities and compatibility with the labeling system employed. For, example, solid phases used with fluorescent labels should have sufficiently low background fluorescence to allow signal detection.

Following attachment of a specific capture agent, the surface of the solid support may be further treated with materials such as serum, proteins, or other blocking agents to minimize non-specific binding.

SARS-CoV-2 specific T cells may be made by various methods.

In one preferred method, PBMCs or hematopoietic cells that contain or can differentiate into T cells are isolated from blood of a donor whose immune system has been exposed to SARS-CoV-2 such as a subject who is or has been infected by SARS-CoV-2 or a convalescent patient.

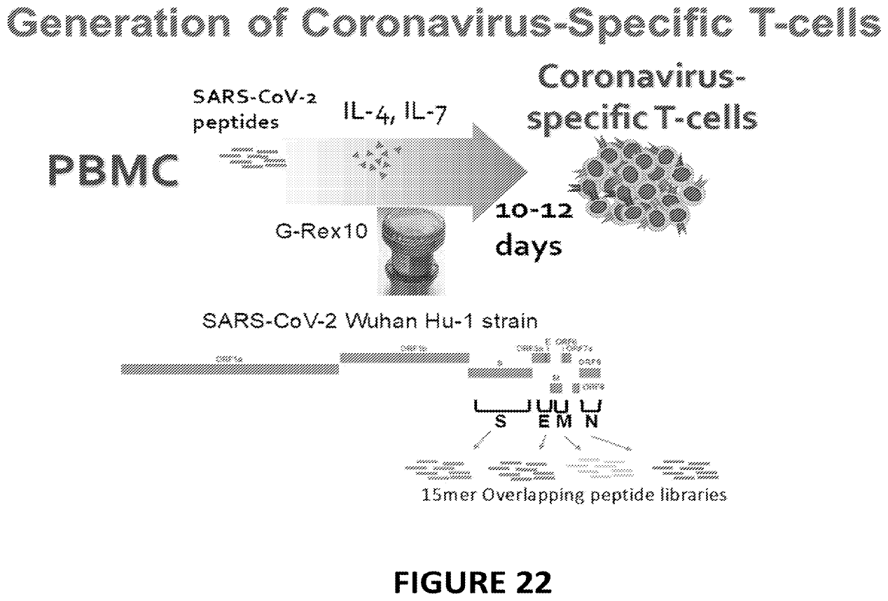

The isolated PBMCs or cells are contacted with a peptide library spanning the sequence one or more SARS-CoV-2 antigens or alternatively contacted with one or more peptide epitopes of SARS-CoV-2 peptide such as those described by SEQ ID NOS: 1-524. The PBMCs are then resuspended and incubated in a medium containing IL-4 and IL-7. These cytokines and culture medium may be replenished during a period of incubation of the PBMCs. FIG. 22 schematically describes an embodiment of this method that uses a library of SARS-CoV-2 overlapping peptides to prime and expand SARS-CoV-2 specific T cells.

After incubation, for example, after 7-12 days, the cells are harvested and evaluated for antigen specificity.

In some embodiments subsets of the harvested cells (SARS-CoV-2-specific T cells or “CSTs”) may be restimulated by contacting them with irradiated autologous PMBCs in the presence of IL-4 and IL-7, which may be replenished during a subsequent period of incubation.

In some embodiments, the initial period of incubation in IL-4 and IL-7 is 5-9 days after which the IL-4 and IL-7 are replenished, preferably on day 7. In some embodiments, the initial incubation ranges from 8-12 days, preferably 10 days, after which the cells are harvested and evaluated for antigen-specificity.

In preferred embodiments, culturing may be continued beyond day 12 provided that the cells are restimulated to avoid loss of antigen specificity. In some embodiments, the subset of harvested cells are restimulated up to Day 18-24, preferably up to Day 21, and the IL-4 and IL-7 are replenished on Day 14-20, preferably on day 17.

Concentrations of IL4 and IL-7 may be selected by skilled experts, for example, by validation of a range of different concentrations or dose-response testing. In some embodiments IL-4 concentration ranges from 10, 20, 50, 100, 200, 500 to 1,000 IU/mL, preferably about 400 IU/mL, and IL-7 concentration ranges from 1, 10, 20, 50 to 100 ng/mL, preferably about 10 ng/mL.

Peptides may be applied at a ratio of about 0.001, 0.002, 0.005, 0.01, 0.02, 0.05, 0.1, 0.2, 0.5, 1, 2, 5 to 10 μg antigen per 15×10⁶ PBMCs.

During restimulation, a ratio of 10:1, 4:1, 2:1, 1:1, 1:2, 1:4, to 1:10 irradiated PBMCs to CSTs may be used. These and other ranges appearing herein include all intermediate subranges and values.

In some embodiments, this method further comprises separating or characterizing a population of antigen-specific T cells into subpopulations enriched for CD4⁺ T cells, CD8⁺ T cells, CD44 (high) cells, or enriched from another T cell phenotype.

This method may also further comprise administering the SARS-COV-2 antigen-specific T-cells to a subject in need thereof, or banking or storing said antigen-specific T-cell for later administration.

In some instances, a non-naïve donor will have antibodies to one or more coronavirus antigens that cross-react with SARS-CoV-2 antigens which may contain conserved epitopes.

In an alternative embodiment, the donor may be naïve to one or more SARS-CoV-2 antigens, a donor who has not been previously infected with SARS-CoV-2, a donor who has not been vaccinated for SARS-CoV-2, or a donor of naïve stem or cord blood cells.

In one embodiment, the donor has antibody levels to one or more SARS-CoV-2 antigens which are greater than a control value from subject(s) uninfected or by SARS-CoV-2.

In another embodiment, the donor has antibody levels to one or more SARS-CoV-2 antigens which are no more than a control value from subject(s) uninfected or for SARS-CoV-2.

In another embodiment, the donor is or has convalesced from SARS-CoV-2 infection.

In another embodiment, the donor has been immunized to at least one SARS-CoV-2 antigen.

In another embodiment, the donor has not been previously exposed to SARS-CoV-2 or said donated cells are naïve to one or more SARS-CoV-2 antigens.

Another method for producing SARS-COV-2 antigen-specific T cells, which may be used with naïve donor cells, comprises:
(a) dividing mononuclear cells from a donor into two portions;
(b) contacting a first portion of said sample with PHA or another mitogen and, optionally with IL-2, to produce ATCs (“activated T cells”) and treating the ATCs with radiation or another agent to inhibit their outgrowth;
(c) separating T-cells and T-cell precursor cells, which may be nonadherent cells, CD3⁺ cells, from dendritic cells and dendritic precursor cell, which may be adherent cells, CD11C⁻ or CD14 cells;
(d) cryopreserving or otherwise reserving the non-adherent cells,
(e) contacting the adherent cells in the second portion with IL-4 and GM-CSF or other cytokine(s) and/or other agent(s) that generate and mature dendritic cell and with at least one SARS-COV-2 peptide antigen of SEQ ID NOS: 1-524 or a SARS-CoV-2 peptide library to produce antigen-presenting dendritic cells that present the at least one peptide antigen, and treating said antigen-presenting dendritic cells with radiation or another agent sufficient to inhibit their outgrowth;
(f) contacting the reserved non-adherent cells from (d) with the dendritic antigen presenting cells produced in (e) in the presence of IL-7 and IL-15 and
   optionally other cytokines, to produce virus- or other antigen-specific T-cells that recognize the at least one peptide antigen;
(g) contacting SARS-COV-2 antigen-specific T-cells produced by (f) with the ATCs of (b) in the presence of the at least one peptide antigen in the presence of K562 cells or other accessory cells and in the presence of IL-15; optionally, repeating (g) one or more times;
(h) recovering antigen-specific T-cells that recognize the at least one SARS-COV-2 peptide antigen.

In one embodiment of this method, a population of T cells or CD14⁻ cells, may be further contracted with IL-21 during their exposure to peptide-loaded CD14⁺ dendritic cells to facilitate T cell priming.

One embodiment further comprises administering said SARS-COV-2 antigen-specific T-cells to a subject in need thereof, or banking or storing said antigen-specific T-cell for later administration.

This method may also further comprise separating or characterizing antigen-specific T cells into subpopulations enriched for CD4⁺ T cells, CD8⁺ T cells, CD44 (high) cells, or enriched for another T cell phenotype.

In some embodiments, the donor has antibody levels to one or more SARS-CoV-2 antigens which are greater than a control value from subject(s) uninfected by SARS-CoV-2 or from unvaccinated subjects.

In other embodiments, the donor has antibody levels to one or more SARS-CoV-2 antigens that are no more than a control value from subject(s) uninfected by SARS-CoV-2 or from unvaccinated subjects.

In some embodiments, the donor is convalescing or has convalesced from SARS-CoV-2 infection.

In other embodiments, the donor has been immunized to at least one SARS-CoV-2 antigen such as S, N, membrane or envelope antigens.

In other embodiments, the donor has not been previously exposed to SARS-CoV-2 or vaccinated to this virus. The donated cells may be naïve to one or more SARS-CoV-2 antigens, such as S, N, membrane or envelope antigens.

In some embodiments, an alternate cytokine cocktail may be used to rapidly expand CSTs, for example, a cocktail containing IL-7 and IL-15 instead of IL-4 and IL-7, may be used for a 10-12 day expansion of SARS-CoV-2 specific T cells. This cocktail is effective in rapid expansion of SARS-CoV-2 specific T cells using otherwise identical or highly similar methods, with increased final yields of virus-specific T cells at 10-12 days.

SARS-COV-2 specific T cells may be produced from PBMCs or hematopoietic cells of naïve subject, such as cord blood or from a subject not exposed to SARS-CoV-2, or from non-naïve subjects, such as a subject who has an active infection with SARS-CoV-2, who has recovered from SARS-COV-2 infection, a subject who has been vaccinated against SARS-CoV-2, or a subject who has antibodies or T cells that recognize SARS-CoV-2 antigens.

In some embodiments, the methods disclosed herein use PBMCs, stem cells, pre-T cells, or cord blood, from a partially histocompatible sibling, parent, son or daughter, grandparent, grandson or granddaughter, first or second cousin, or other blood relative.

In other embodiments, T cells may be obtained from autologous cells. Those skilled in the art may select an appropriate match by minimizing mismatches of HLA type-I genes (e.g. HLA-A, HLA-B, or HLA-C) which increase the risk of graft rejection, and/or by minimizing the mismatches of an HLA type 11 gene (e.g. HLA-DR or HLA-DOBI) which increase the risk of graft-versus-host disease. Typically, antigen-specific T cells are produced from naïve cells that share at least 1, 2, 3, 4, 5, 6, 7, 8, 9, 10, 11 or 12 HLA alleles (e.g., HLA-A, HLA-B, HL4-C, HLA-DPA1, HLA-DPB1, HL4-DQA1, HLA-DQB1, HLA-DRA, and HL4-DRB1) with a prospective donor.

Typically cells, such as PBMCs, stem cells, or cord blood cells, from a donor will match at least 1, 2, 3, 4, 5, 6, 7, or 8 HLA alleles of a recipient. Thus, banked antigen-specific T cells can be labelled with the HLA background of the donor cells so that an appropriate match may be made to the HLA background of a recipient. Thus, another aspect of the invention is directed to a storage bank comprising cryopreserved samples of the T cell composition as disclosed herein, wherein each sample identifies the donor and the donor's HLA background. The bank may also contain separate samples of T cells that recognize particular antigens, such as membrane, spike or nucleocapsid peptide antigens. Each deposit of antigen-specific T cells in the bank may be classified according to antigen-specificity of the T cells, specify the HLA background of the deposited cells, as well as the source of the cells used to produce the antigen-specific T cells. Such a bank provides a convenient off-the-shelf selection for rapid administration of antigen-specific T cells to a subject in need thereof.

In some embodiments, in the methods described herein, the mononuclear cells are taken from a donor who has been infected with SARS-CoV-2, from a donor whose immune system has been vaccinated or otherwise exposed to an immunogenic SARS-CoV-2 antigen, or wherein the mononuclear cells comprise memory T cells to SARS-CoV-2.

In other embodiments, the mononuclear cells are from a donor whose immune system has not been exposed to SARS-CoV-2 or who does not have memory cells to SARS-CoV-2.

In some embodiments, the mononuclear cells are taken from cord blood.

In some embodiments, the mononuclear cells are prepared from buffy coat cells, bone marrow cells, or cells from the spleen.

In some embodiments, the at least one peptide antigen comprises a peptide having a length that is restrictable by an MHC class I or class II antigen and which comprises at least one amino acid sequence of SEQ ID NOS 1-524.

In some embodiments, the at least one peptide antigen comprises 1, 2, 3, 4, 5, 6, 7, 8, 9, 10 or more peptides comprising different amino acid sequences of SEQ ID NOS: 1-524. These peptides may be from the same SARS-CoV-2 antigen or from different antigens.

In other embodiments, the methods described above may employ a library of overlapping peptides that overlap substantially the length of at least one SARS-CoV-2 antigen. Overlapping peptide pools or libraries may be constructed or commercially ordered, for example from A&A Peptides, San Diego, CA. SARS-CoV-2 Pepmix libraries available as of the filing date from JPT are hereby incorporated by reference to hypertext transfer protocol secure://www.jpt.com/products/pepmix-peptide-pools/(incorporated by reference). These include EMPS-WCPV-NCAP-1, EMPS-WCPV-S-1 and EMPS-WCPV-VEMP-1.

In some embodiments, the overlapping peptide libraries are produced using the sequences of the NC_045512.2 SARS-CoV-2 reference sequence. In other embodiments other known reference sequences may be used to produce the overlapping peptide pools or libraries.

In some embodiments, the at least one peptide antigen is restrictable by an HLA-DRB1, HLA-DRB3, HLA-DRB4, HLA-DRB-5, HLA-DQA1, HLA-DQB1, HLA-DPA1, or HLA-DPB1 antigen of said donor or of a recipient.

In another embodiment, said at least one peptide antigen is restrictable by an HLA-A, HLA-B or HLA-C molecule or complex of said donor or recipient.

Another aspect of this technology is directed to a composition, comprising, consisting essentially of, or consisting of a population of SARS-CoV-2 specific T cells that recognize one or more peptide antigens of SARS-CoV-2 described by SEQ ID NOS: 1-524.

The term “composition” or “T cell composition” refers to T cells per se as well as T cells in suspending buffer or medium or T cells attached to a solid substrate or other material.

T cells that recognize SARS-CoV-2 antigens may be further isolated or purified based on cell surface markers. T cell phenotypes include cells with one or more of the following markers: CD4+, CD8+, CD4+/CD25+, CD45RO+, CD27+, CD28+, and/or PD1. T cell phenotypes include CD4+CD8+; CD27+CD28+ and CD4+, CD45RO+ and CD27+. Cells with undesired phenotypes may be removed or separated from desired SARS-CoV-2 recognizing T cells (CSTs) using methods known in the art. This procedure permits isolation or purification of a subpopulation of T cells with particular characteristics such as T cell memory, helper T cell function or cytotoxic T cell function.

T cells may be separated from other cellular and non-cellular components of blood or other biological fluid, or from other components of a culture medium or buffer. For example, they may be isolated from red blood cells on a density gradient and recovered from a buffy coat layer or may be sorted using a cell sorter. T cells may also be separated by filtration or centrifugation from other culture components, such as culture medium containing particular cytokines.

In one embodiment, the T cell composition is made by contacting PBMCs with peptides described by SEQ ID NOS: 1-524 or with peptide libraries of SARS-CoV-2 antigens, followed by culturing in a medium containing IL-4 and IL-7 as described above.

In another embodiment, the T cell composition comprises SARS-CoV specific T cells which recognize at least 1, 2, 3, 4, 5, 6, 7, 8, 9, 10 or more different peptide antigens of SARS-CoV-2 described by SEQ ID NOS: 1-524.

In another embodiment, the T cell composition comprises SARS-CoV specific T cells that recognize at least three different peptide antigens of SARS-CoV-2 described by SEQ ID NOS: 1-524, wherein said different peptide antigens comprise peptide antigens from S protein, N protein, membrane protein, or envelope protein.

In another embodiment, the SARS-CoV specific T cells recognize at least three different peptide antigens of SARS-CoV-2 described by SEQ ID NOS: 1-524, wherein said different peptide antigens comprise at least three peptide antigens from S protein, at least three different peptide antigens from N protein or at least three different peptide antigens from membrane protein.

In some embodiments the T cells comprise or substantially comprise helper T cells or CD4⁺ T cells which recognize the peptide antigen in the context of an MHC Class 2 molecule.

In other embodiments, the T cells comprise or substantially comprise cytotoxic T cells or CD8⁺ T cells which recognize the peptide antigen in the context of an MHC Class 1 molecule.

In one embodiment, the T cell composition recognizes a peptide antigen comprising Leu Arg Gly His Leu Arg Ile Ala Gly His His Leu Gly Arg Cys (SEQ ID NO: 53), Leu Arg Ile Ala Gly His His Leu Gly Arg Cys Asp Ile Lys Asp (SEQ ID NO: 54), Ser Arg Thr Leu Ser Tyr Tyr Lys Leu Gly Ala Ser Gin Arg Val (SEQ ID NO: 60), Ser Tyr Tyr Lys Leu Gly Ala Ser Gln Arg Val Ala Gly Asp Ser (SEQ ID NO: 61), or Leu Gly Ala Ser Gin Arg Val Ala Gly Asp Ser Gly Phe Ala Ala (SEQ ID NO: 62), which correspond to a segment of the membrane protein.

In one embodiment, the T cell composition recognizes a peptide antigen comprising Lys Pro Arg Gin Lys Arg Thr Ala Thr Lys Ala Tyr Asn Val Thr (SEQ ID NO: 450), Ala Phe Phe Gly Met Ser Arg Ile Gly Met Glu Val Thr Pro Ser (SEQ ID NO: 464); Met Ser Arg Ile Gly Met Glu Val Thr Pro Ser Gly Thr Trp Leu (SEQ ID NO: 465) or Gly Met Glu Val Thr Pro Ser Gly Thr Trp Leu Thr Tyr Thr Gly (SEQ ID NO: 466) which corresponds to a segment of the nucleocapsid protein.

In one embodiment, the T cell composition recognizes a peptide antigen comprising Pro Phe Phe Ser Asn Val Thr Trp Phe His Ala Ile His Val Ser (SEQ ID NO: 84), Asn Val Thr Trp Phe His Ala Ile His Val Ser Gly Thr Asn Gly (SEQ ID NO: 85), Ser Lys His Thr Pro Ile Asn Leu Val Arg Asp Leu Pro Gin Gly (SEQ ID NO: 121); Pro Ile Asn Leu Val Arg Asp Leu Pro Gin Gly Phe Ser Ala Leu (SEQ ID NO: 122), or Tyr Asn Tyr Leu Tyr Arg Leu Phe Arg Lys Ser Asn Leu Lys Pro (SEQ TD NO: 182) which corresponds to a segment of the S protein.

In another embodiment, the T cell composition may be deposited and cryogenically stored in a storage bank wherein each sample identifies the donor and the donor's HLA

### BACKGROUND

Another aspect of this technology is a peptide complex or peptide construct comprising at least one peptide described by SEQ ID NOS: 1-524 or antigenic or immunogenic fragment thereof.

A peptide complex may be formed by non-covalently binding a peptide to another moiety such as a carrier, adjuvant or substrate. In some embodiments a peptide is altered by non-covalently binding it to a carrier, adjuvant or substrate such as to PEG, BSA, or KLH. A peptide of SEQ ID NOS: 1-524 may form a non-covalent complex with an MHC class I or class II molecule or a complex with a cell membrane or cell comprising MHC class 1 or 2 molecules.

A peptide construct may be formed by covalently modifying a peptide of SEQ ID NOS: 1-524 to alter its pharmacokinetic or pharmacodynamics properties compared to a corresponding unmodified peptide. In some embodiments, the peptide has been covalently modified at its N- or C-terminal to form a non-natural peptide complex. In another embodiment, the peptide has been modified by covalent conjugation to PEG, BSA, KLH, or to another carrier, adjuvant or substrate. In other embodiments, the peptide comprises at least one peptide described by SEQ ID NOS: 1-524 which has been modified by replacement of at least 1, 2, 3, 4, 5 or more amino acid residues with a corresponding D-amino acid residue.

Another aspect of the invention is directed to a composition comprising at least one peptide described by SEQ ID NOS: 1-524 or a peptide construct thereof, such as a modified peptide having the same amino acid sequence, a peptide analog having 1 or 2 deletions, insertions or substitutions into a peptide described by SEQ ID NOS: 1-524; and a pharmaceutically acceptable carrier or excipient.

The peptide composition disclosed herein may comprise 1, 2, 3, 4, 5, 6, 7, 8, 9, 10 or more peptides described by SEQ ID NOS: 1-524 having different amino acid sequences. These sequences may be from the same SARS-CoV-2 antigen or from different SARS-CoV-2 antigens.

The composition may further comprise an adjuvant or be formulated as a peptide-based vaccine. Thus, a further aspect of the invention relates to an immunogen or vaccine comprising the peptide epitopes of SEQ ID NOS: 1-524 described herein, and, optionally a suitable excipient and/or adjuvant. In one embodiment a polypeptide or polypeptide fragment of a SARS-CoV-2 spike (S), nucleocapsid (N), membrane or envelope protein may be bound to an immunogenic carrier such as BSA, KLH, tetanus toxoid or other immunogenic carrier; or may be incorporated into a liposome.

A liposome may be formulated to contain lipid A, muramyldipeptide or IL-1 as immunomodulators. Types and formulations of liposomes suitable for carriers of immunogens are known in the art and are incorporated by reference to Kaskin, K P, et al., UKR BIOKHIM ZH (59(4):100-107 (1978) and to Chapter 4, Liposomal-based therapeutic carriers for vaccine and gene delivery, M. Rahman, et al., NANOTECHNOLOGY-BASED APPROACHES FOR TARGETING AND DELIVERY OF DRUGS AND GENES, 2017, Pages 151-166.

In general, the peptide-, peptide construct-, cellular-, and nucleic acid-based materials described herein may be incorporated into a composition. Typically, such a composition will include a pharmaceutically acceptable excipient or carrier and may further contain an adjuvant or other active agent.

The term carrier encompasses any excipient, binder, diluent, filler, salt, buffer, solubilizer, lipid, stabilizer, or other material well known in the art for use in pharmaceutical formulations, for example, for intravenous administration a carrier may be sodium chloride 0.9% or mixtures of normal saline with glucose or mannose. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005, which is incorporated herein by reference in its entirety.

An adjuvant is a pharmacological or agent that modifies the effect of other agents. Adjuvants may be added to the materials disclosed herein, such as peptides, peptide constructs, cells and nucleic acids to boost the humoral or cellular immune responses and produce more intense or longer-lasting immunity, thus minimizing the dose of material needed.

Adjuvants that may be compounded with, or otherwise used along with the peptide, peptide construct, cell, or nucleic acids disclosed herein include, but are not limited to, inorganic compounds including alum, aluminum hydroxide, aluminum phosphate, calcium phosphate hydroxide; mineral oil or paraffin oil; bacterial products or their immunologically active fractions, such as those derived killed Bordatella pertussis, Mycobacterium bovis, or bacterial toxoids; organics such as squalene; detergents such as Quil A, saponins such as Quillaja, soybean or polygala senega; cytokines such as IL-1, IL-2 or IL-12; Freund's complete adjuvant or Freund's incomplete adjuvant; and food based oils like Adjuvant 65, which is a product based on peanut oil. Those skilled in the medical or immunological arts may select an appropriate adjuvant based on the type of patient and mode of administration of the materials described herein.

For therapeutic purposes, formulations for parenteral administration can be in the form of aqueous or non-aqueous isotonic sterile injection solutions or suspensions. The term parenteral, as used herein, includes intravenous, intravesical, intraperitoneal, subcutaneous, intramuscular, intralesional, intracranial, intrapulmonal, intracardial, intrasternal, and sublingual injections, or infusion techniques. These solutions and suspensions can be prepared from sterile powders or granules having one or more of the carriers or diluents mentioned for use in the formulations for oral administration, preferably in a digestion-resistant form such as an enteric coating. The active ingredient can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Injectable preparations, for example, sterile injectable aqueous or oleaginous suspensions can be formulated according to the known art using suitable dispersing or wetting ingredients and suspending ingredients. The sterile injectable preparation can also be a sterile injectable solution or suspension in a non-toxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids, such as oleic acid, find use in the preparation of injectables. Dimethyl acetamide, surfactants including ionic and non-ionic detergents, polyethylene glycols can be used. Mixtures of solvents and wetting ingredients such as those discussed above are also useful.

Administration to the respiratory system may be accomplished using a drug delivery device such as a nebulize to administer a peptide, peptide construct, cell or nucleic acid as disclosed herein, in an inhalable form. Nebulizers for treatment of cystic fibrosis, asthma, COPD and other respiratory diseases are known and incorporated by reference to hypertext transfer protocol secure://en.wikipedia.org/wiki/Nebulizer. These include soft mist inhalers, jet nebulizers, ultrasonic wave nebulizers, and nebulizers using vibrating mesh technology.

A metered-dosage inhaler is another drug delivery device that delivers a selected or metered amount of a medication, such as the peptide, peptide constructs, cells expressing a peptide epitope disclosed herein, or nucleic acid (including vectors and DNA or RNA constructs expressing a peptide epitope) materials disclosed herein. Typically, this device produces and releases an aerosol of micrometer-sized particles that are inhaled. In some cases, the particles may be a dry powder in others as a mist or in a semiliquid form. Metered-dose inhalers and their various components, propellants, excipients and other elements are described by and incorporated by reference to hypertext transfer protocol secure://en.wikipedia.org/wiki/Metered-dose_inhaler. An inhalable composition may be formulated in the form of a hydrofluoroalkane inhaler or HFA (metered dose inhaler or MDI), dry powder inhaler (DPI), or as a nebulizer solution.

Another aspect of the invention is directed to a method for inducing immunity to SARS-COV-2 comprising administering to a subject in need thereof at least one peptide antigen, peptide complex, peptide conjugate, composition, or vaccine which comprises a peptide sequence of any one of SEQ ID NOS: 1-524.

In some embodiments, the method comprises administering 1, 2, 3, 4, 5, 6, 7, 8, 9, 10 or more peptide antigens of SEQ ID NOS: 1-524. These peptide antigens may be from the same or different SARS-CoV-2 antigens.

The route of administration of peptide or peptide-construct-based vaccine may be determined by one skilled in the art. In some embodiments, the at least one peptide antigen, peptide complex, peptide conjugate, composition or vaccine is administered orally, sub-buccally, or sublingually. In other embodiments the at least one peptide, peptide conjugate, composition or vaccine is administered into the nose, sinus, eyes, or upper or lower respiratory systems of the subject. In other embodiments, the at least one peptide, peptide conjugate, composition or vaccine is administered parenterally, for example, intravenously, intradermally, or intramuscularly.

Another aspect of the invention is directed to a method for inducing immunity to SARS-COV-2 comprising administering a nucleic acid or a modified nucleic acid encoding at least one peptide antigen of SEQ ID NOS: 1-524 to a subject in need thereof.

In one embodiment, the nucleic acid is RNA or modified RNA and in another embodiment the nucleic acid is DNA or modified DNA.

The nucleic acid used in this method may encode 1, 2, 3, 4, 5, 6, 7, 8, 9, 10 or more peptides of SEQ ID NOS: 1-524.

Nucleic acids, RNA or DNA or their analogs encoding IBV antigens, may be administered by transfection or infection using methods known in the art, including but not limited to the methods described I, and incorporated by reference to, McCaffrey et al., Nature, 2002, 418(6893), 38-9 (hydrodynamic transfection); Xia et al., NATURE BIOTECHNOL., 2002, 20(10), 1006-10 (viral mediated delivery); or Putnam, AM. J. HEALTH SYST PHARM. 1006, 53(2), 151-160, erratum at AM. J. HEALTH SYSTEM PHARM. 1996, 53(3), 325. Therapeutic nucleic acids encoding the peptide epitopes disclosed herein can also be administered by known methods such via a DNA or RNA vaccine.

This method as well as the other treatment methods disclosed herein may be used to treat patients at risk of SARS-CoV-2 infection including subjects less than 1, 2, 3, 4, 5, 6, 7, 8, 9, 10, 15, 18, 21, 30, 35, 40, 45, 50, 55, 60, 65, 70, 75, 80, 85, 90, 95, 100, or >100 years old. Other treatable subjects include smokers, drug abusers, those with COPD or respiratory diseases or conditions, diabetics, those with high blood pressure, heart disease or coronary artery disease, or subjects who are immune suppressed.

Another aspect of this invention is directed to a nucleic acid, such as RNA, DNA or modified RNA or DNA, encoding at least one peptide described by SEQ ID NOS: 1-524. In some embodiments, the nucleic acid may encode 1, 2, 3, 4, 5, 6, 7, 8, 9, 10 or more peptide sequences according to SEQ ID NOS: 1-524. Nucleic acids encoding multiple peptide sequences may encode peptide sequences from a single SARS-CoV-2 antigen or from 2, 3 or more SARS-CoV-2 antigens.

The nucleic acid may further be incorporated into a vector or DNA construct, such as one capable of expressing the peptide or a protein containing the peptide in a cell. Some vectors or DNA constructs will contain promoters or ribosome binding sequences to permit expression of the peptide sequence in a host cell.

The DNA construct or vector may be configured as a vaccine for expression of the peptide sequence or a longer peptide comprising said peptide sequence in a host cell. RNA and DNA-based vaccination methods include intramuscular injection, administration by gene guns, bio injectors, and skin patches as well as needle-free methods such as the micro-particle DNA vaccine technology disclosed in U.S. Pat. No. 6,194,389 and transdermal needle-free vaccination with powder-form vaccine as disclosed in U.S. Pat. No. 6,168,587, both incorporated by reference. Additionally, mucus membrane delivery is possible, as described in, inter alia, Hamajima et al., CLIN. IMMUNOL. IMMUNOPATHOL., 1998, 88(2), 205-10. Liposomes (e.g., as described in U.S. Pat. No. 6,472,375) and microencapsulation can also be used. Biodegradable targetable microparticle delivery systems can also be used, e.g., as described in U.S. Pat. No. 6,471,996. All of the above documents are incorporated by reference for the methods and reagents they disclose. Other vectors and delivery methods for nucleic acid based vaccines are known and are incorporated by reference to hypertext protocol secure://en.wikipedia.org/wiki/DNA vaccine (last accessed Feb. 26, 2021). One skilled in the art may select or design a vector suitable for delivery of a peptide epitope or antigen disclosed herein. In one embodiment, the vector or DNA construct is incorporated into an autologous or allogeneic human cell, such as a cell that expresses at least one major histocompatibility antigen that can restrict the peptide.

Another aspect of the invention is directed to a method for determining whether a subject has been exposed to SARS-CoV-2 comprising contacting lymphocytes from a subject with antigen presenting cells loaded with at least one peptide described by SEQ ID NOS: 1-524, and measuring activation of the lymphocytes compared to control cells not loaded with said at least one peptide, and selecting a subject who has been exposed to SARS CoV-2 when said lymphocytes are activated compared to control cells not contacted with said peptide.

In another embodiment of this method, the at least one peptide antigen comprises Leu Arg Gly His Leu Arg Ile Ala Gly His His Leu Gly Arg Cys (SEQ ID NO: 53), Leu Arg Ile Ala Gly His His Leu Gly Arg Cys Asp Ile Lys Asp (SEQ ID NO: 54), Ser Arg Thr Leu Ser Tyr Tyr Lys Leu Gly Ala Ser Gin Arg Val (SEQ ID NO: 60), Ser Tyr Tyr Lys Leu Gly Ala Ser Gln Arg Val Ala Gly Asp Ser (SEQ ID NO: 61), or Leu Gly Ala Ser Gln Arg Val Ala Gly Asp Ser Gly Phe Ala Ala (SEQ ID NO: 62), which corresponds to a segment of the membrane protein.

In another embodiment of this method the at least one peptide antigen comprises Lys Pro Arg Gln Lys Arg Thr Ala Thr Lys Ala Tyr Asn Val Thr (SEQ ID NO: 450), Ala Phe Phe Gly Met Ser Arg Ile Gly Met Glu Val Thr Pro Ser (SEQ ID NO: 464); Met Ser Arg Ile Gly Met Glu Val Thr Pro Ser Gly Thr Trp Leu (SEQ ID NO: 465) or Gly Met Glu Val Thr Pro Ser Gly Thr Trp Leu Thr Tyr Thr Gly (SEQ ID NO: 466) which corresponds to a segment of the nucleocapsid protein.

In another embodiment of this method the at least one peptide antigen comprises Pro Phe Phe Ser Asn Val Thr Trp Phe His Ala Ile His Val Ser (SEQ ID NO: 84), Asn Val Thr Trp Phe His Ala Ile His Val Ser Gly Thr Asn Gly (SEQ ID NO: 85), Ser Lys His Thr Pro lie Asn Leu Val Arg Asp Leu Pro Gln Gly (SEQ ID NO; 121); Pro Ile Asn Leu Val Arg Asp Leu Pro Gln Gly Phe Ser Ala Leu (SEQ ID NO: 122), or Tyr Asn Tyr Leu Tyr Arg Leu Phe Arg Lys Ser Asn Leu Lys Pro (SEQ ID NO: 182) which corresponds to a segment of the S protein.

Another embodiment of the invention comprises a kit for detecting T cells which recognize SARS-CoV-2 comprising one or more peptides described by SEQ ID NOS: 1-524, and optionally, fluorophore-conjugated antibodies to CD4, CD8, TCRαβ, CXCR3, CXCR5, CCR6, CD127, CD25, CD56 or other cell surface markers and/or components of an IFN-γ ELIS spot assay. Other kit components and methods of detection of SARS-CoV-2 specific T cells are known in the art and are incorporated by reference to Phetsouphanh C, et al, INT J MOL SCI. 2015 Aug. 12; 16(8):18878-93. doi: 10.3390/ijms160818878.

### EXAMPLES

Donors. Peripheral blood mononuclear cells (PBMCs) from volunteers, both healthy and those with presumed or documented COVID-19 infection, were obtained from Children's National Hospital (Washington, DC) and the National Institutes of Health under informed consent approved by the Institutional Review Board of both institutions in accordance with the Declaration of Helsinki.

Generation of SARSCoV-2—specific T cells. Evaluated T-cell products included SARS-CoV-2-specific T cells (CSTs), manufactured from PBMCs of seropositive and seronegative volunteers.

VSTs (virus specific T cells) were produced using a rapid expansion protocol. Briefly, PBMCs were pulsed with a mix of overlapping peptide pools encompassing viral structural proteins (1 jpg/antigen per 15×10⁶ PBMCs) for 30 minutes at 37° C.

Peptide libraries of 15-mers with 11 amino acid overlaps encompassing the spike, membrane, nucleocapsid, and envelope proteins were generated (A&A Peptide, San Diego, CA) from the SARS-CoV-2 reference sequence (NC_045512.2), and were pooled equally by mass and reconstituted to a working concentration of 1 mg/mL.

After incubation, cells were resuspended with interleukin-4 (IL-4; 400 IU/mL; R&D Systems, Minneapolis, MN) and IL-7 (10 ng/mL: R&D Systems) in CTL media consisting of 45% RPMI (GE Healthcare, Logan, UT), 45% Click medium (Irvine Scientific, Santa Ana, CA), 10% fetal bovine serum (FBS), and supplemented with 2 mM GlutaMax (Gibco, Grand Island, NY) according to our GMP-compliant standard operating procedures.

Cytokines were replenished on day 7.

On day 10, cells were harvested and evaluated for antigen specificity and functionality.

A subset of samples was restimulated with autologous PBMCs that were pulsed with the viral peptide libraries, irradiated at 75 Gy, and cocultured with the CSTs at a ratio of 1:4 (CSTs to PBMCs). These restimulated cells were incubated in IL-4 (400 TU/mL) and IL-7 (10 ng/mL), with cytokines replenished at day 17, and harvested at day 21 for further testing.

Isolation and maintenance of SARSCoV-2-specific T-cell clones. Membrane and spike-specific T cells were isolated from frozen VSTs using an interferon-γ (IFN-γ) capture assay protocol. Briefly, VSTs were thawed, washed in warm X-VIVO-15, and resuspended at a concentration of 1×10⁷ cells/mL.

VSTs were stimulated for 3 hours with overlapping peptide pools encompassing viral antigens to spike and membrane to a final concentration of 1 μg/mL.

T cells producing IFN-g in response to this stimulation were enriched using the IFN-γ Secretion Detection and Enrichment Kit (130-054-201; Miltenyi Biotec, Bergisch Gladbach, Germany) in accordance with the manufacturer's instructions.

These T cells were plated at a series of dilutions in 96-well plates with irradiated feeder medium (RPMI 1640 supplemented with 10% FBS, L-glutamine, and PenStrep [R-10]) with 1×10⁶ cells/mL 5000 rad irradiated PBMC+50 U/mL IL-2+10 ng/mL IL-15+0.1 μg/mL each of anti-CD3 (Ultra-LEAF purified anti-human CD3 antibody clone OKT3; BioLegend, San Diego, CA) and anti-CD28 (Ultra-LEAF purified Anti-human CD28 antibody clone 28.2; BioLegend).

Membrane and spike-specific T-cell clones were expanded biweekly with irradiated feeder medium.

One month later, colonies were selected from the lowest dilution plates with positive wells (<1/3 of wells positive) and screened for responsiveness to membrane or spike peptide pools by intracellular cytokine staining for IFN-g and tumor necrosis factor-α (TNF-α).

IFN-γ, ELISpot assay. Antigen specificity of T cells was measured by IFN-γ enzyme linked immunospot (ELISpot; Millipore, Burlington, MA). T cells were plated at 1×10⁵/well with no peptide, actin (control), or each of the individual SARS-CoV-2 pepmixes (200 ng per peptide per well). Plates were sent for IFN-γ spot-forming cells counting (Zellnet Consulting, Fort Lee, NJ).

Flow cytometry. VSTs were stained with fluorophore-conjugated antibodies against CD4, CD8, TCRαβ, TCRγΔ, CXCR3, CXCR5, CCR6, CD127, CD25, and CD56 (Miltenyi Biotec; BioLegend). All samples were acquired on a CytoFLEX cytometer (Beckman Coulter, Brea, CA). Intracellular cytokine staining was performed as follows: 1×10⁶ VSTs were plated in a 96-well plate and stimulated with pooled pepmixes or individual peptides (200 ng per peptide per well) or actin (control) in the presence of brefeldin A (Golgiplug; BD Biosciences, San Jose, CA) and CD28/CD49d (BD Biosciences) for 6 hours. T-cells were fixed, permeabilized with Cytofix/Cytopermsolution (BDBiosciences), and stained with IFN-γ and TNF-α and IL-2 antibodies (Miltenyi Biotec).

For intracellular flow cytometry of T-cell clones, cells were stimulated with membrane and spike peptide pools to a concentration of 1 μg/mL, and incubated at 37° C. 5% CO₂. After 1 hour, 1 μg/mL of brefeldin A was added to each well, and plates were incubated for another 5 hours. Cells were then washed in 2% FBS phosphate-buffered saline and surface stained with fluorochrome-conjugated antibodies to CD3− Brilliant Violet 785 clone OKT3, CD4-Alexa Fluor 700 clone RPA-T4, CD8-FITC clone RPA-T8, OX40-Brilliant Violet 711 clone Ber-ACT35 (ACT35) (all from BioLegend), CD69-APCeFluor 780 clone FN50, and Fixable Aqua Viability Dye (both from Invitrogen). Cells were fixed, permeabilized using BD Cytofix/Cytoperm solution and stained with anti-IFN-g Brilliant Violet 421 clone 4S. B3, anti-TNF-a PerCP-Cyanine5.5 clone Mab11 (both from BioLegend). Cells were analyzed on an Attune NxT flow cytometer. Data were analyzed with FlowJo X (FlowJo LLC, Ashland, OR).

Epitope mapping. CSTs were tested for specificity to minipools containing 8 to 24 peptides spanning the SARS-CoV2 antigens by IFN-g ELISpot. Cross-reactive pools were analyzed and individual peptides were tested to confirm epitope specificity. In silico predictions of major histocompatibility complex (MHC) restrictions were performed using MARIA (hypertext transfer protocol://maria.stanford.edu) and NetMH-CIIPan (hypertext transfer protocol://www.cbs.dtu.dk/services/NetMHCIIpan-4.0/); Reynisson B, et al., Improved prediction of MHC II antigen presentation through integration and motif deconvolution of mass spectrometry MHC eluted ligand data. J Proteome Res. 2020; 19(6):2304-2315; Chen B, et al. Predicting HL4 class II antigen presentation through integrated deep learning. NAT BIOTECHNOL. 2019; 37(11):1332-1343.

MHC restrictions were narrowed through use of blocking antibodies targeting MHC class H proteins. Briefly, CSTs were incubated were pulsed with 1 mg/mL of spike or membrane peptide pools and blocked with 10 mg/mL of either anti-HLA-DR, anti-HLA-DQ, or anti-HLA-DR, DP, DQ (BioLegend) for 30 minutes. Cells were washed 3 times with R10, and then blocked again with the same concentration of antibodies. After 1 hour, 1 μg/mL of brefeldin A was added to each well, and plates were incubated for another 5 hours. Cells were then washed and stained for surface markers and intracellular cytokines as described previously.

To confirm the restricted HLA allele, CSTs were plated at 1×10⁵ per well with partially HLA-matched phytohemagglutinin treated lymphoblasts (phytohemagglutinin blasts, 25 Gy irradiated) either alone or pulsed with peptide (1 μg/mL), and tested via IFN-γ ELISpot.

Luciferase immunoprecipitation systems for measurement qf SARS-CoV-2 antibodies. Testing for antibodies to spike and nucleocapsid proteins were performed using a luciferase immunoprecipitation system assay as recently described; Burbelo P D, et al., Detection of nucleocapsid antibody to SARS-CoV-2 is more sensitive than antibody to Spike protein in COVID-19 patients. J INFECT DIS. 2020; 222:206-213. Briefly, plasma samples were incubated with spike and nucleocapsid proteins fused to Gaussia and Renilla luciferase, respectively, protein A/G beads were added, the mixture was washed, coelenterazine substrate (Promega) was added, and luciferase activity was measured in light units with a Berthold 165 LB 960 Centro Microplate Luminometer. Antibody levels were reported as the geometric mean level with 95% confidence interval. Cutoff limits for determining positive antibodies in the SARS-CoV-2-infected samples were based on the mean plus 3 standard deviations of the serum values derived from uninfected blood donor controls or by receiver operator characteristics analysis. For some of the data percentages for categorical variables, mean and range, geometric mean, and 95% confidence interval were used to describe the data. Wilcoxon signed-rank tests were used for statistical analysis.

Multiplex cytokine assay. CSTs were plated at 1×10⁵ per well in 96-well plates, stimulated with pooled pepmixes (200 ng/peptide/well) or control actin peptide, and incubated 48 hours. Supernatants were harvested and the cytokine profile analysis was performed using the Bio-plex Pro Human 17-Plex Cytokine Assay Kit (Bio-Rad, Hercules, CA), and read on a MAGPIX system (Luminex, Austin, TX).

Chromium release assay. Phytohemagglutinin blasts were labeled with chromium-51(Perkin Elmer, Waltham, MA) at 10 mCi per 5×10⁵ cells. CST were coplated with 51Cr-labeled, MHC-mismatched irradiated phytohemagglutinin blasts at effector:target ratios between 40:1 and 5:1, and incubated at 37° C. for 4 hours. Maximal release was evaluated by lysis of 51Cr-labeled targets with Triton-X-100. Supernatants were transferred to lumiplates and read on a MicroBeta2 Plate Reader (Perkin Elmer). Specific lysis was calculated as follows: (experimental counts per minute [CPM]−background CPM)/(maximal CPM−background CPM).

Statistical analysis. Statistical analysis was performed in SAS (SAS Institute, Cary, NC). Pearson/Speakman calculations were used for correlations of T-cell and antibody responses of individual antigens, and Pearson X² test was used for binary correlations of T-cell and antibody responses. Graphs were produced in Prism (GraphPad, San Diego, CA). Immunodominance was defined as antigens and/or epitopes that induce statistically significant responses on IFN-γ ELISpot and/or intracellular cytokine staining in comparison with control peptides, and are recognized by multiple individuals.

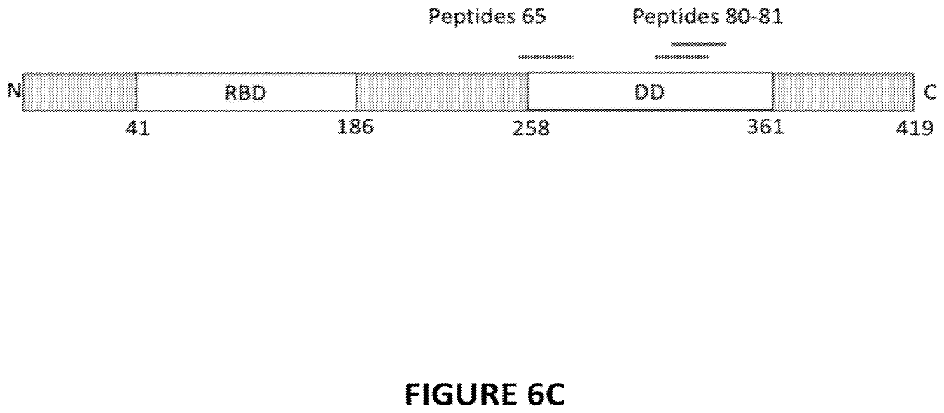

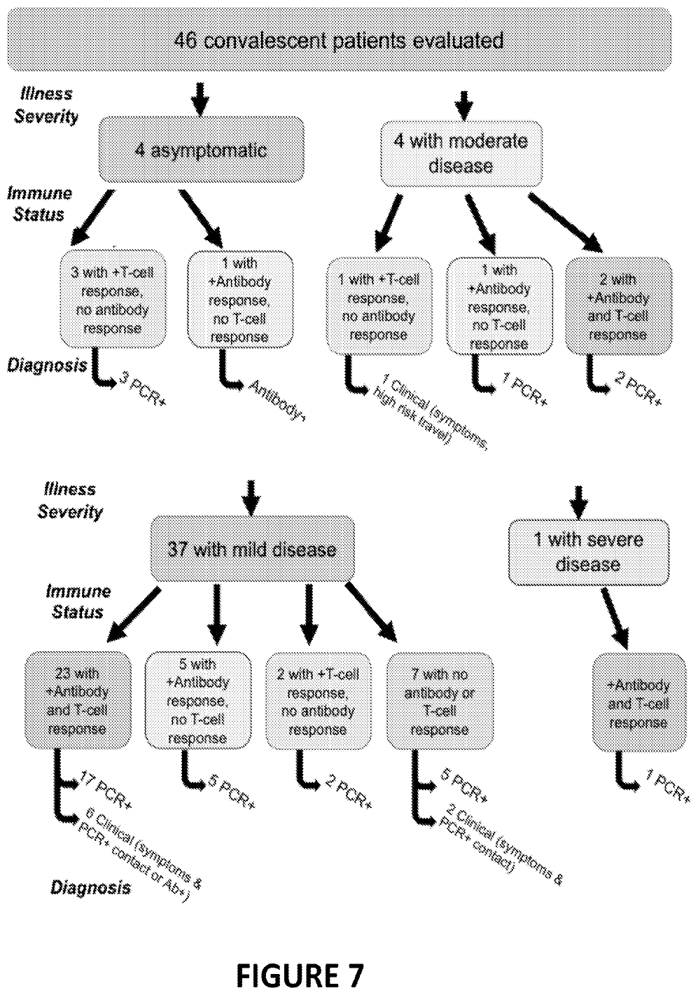

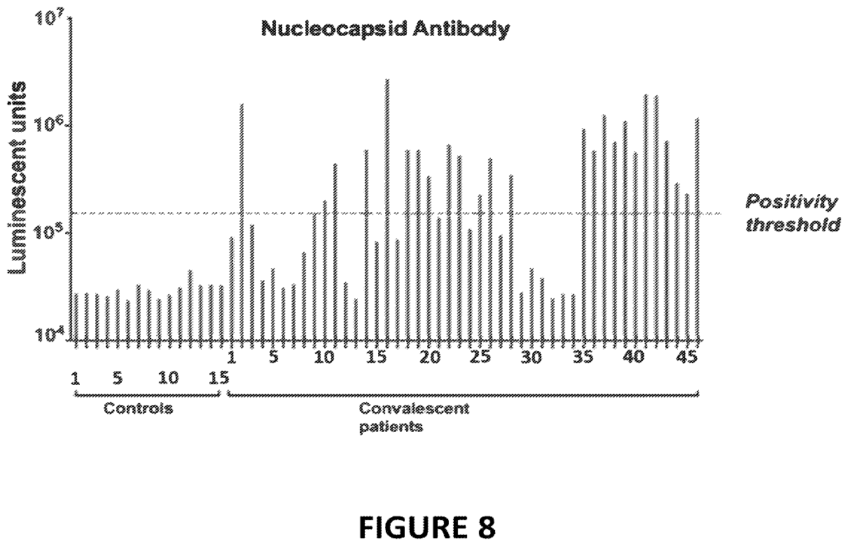

The majority of convalescent patients showed antibody responses to SARS-CoV-2. Forty-six convalescent donors from the eastern and midwestern United States with presumptive recent COVID-19 (36 polymerase chain reaction [PCR]-proven and 10 presumed positive because they were: (1) symptomatic and in close contact with PCR-positive individuals and/or (2) positive for SARS CoV-2 antibody testing) were evaluated at a median time of 36 days after symptom onset (range, 18-111). Median donor age was 34.5 years (range, 20-69). Most patients had mild disease (83%) and 4 were asymptomatic, whereas 4 had moderate disease and 1 had severe disease based on the World Health Organization's SARS-CoV-2 disease severity scale (world wide web.who.int), with a median of 12 days of illness (Table 1; FIG. 7, available on the Blood Web site). Antibody responses were detected in 33 of the 46 convalescent donors (27/46 to spike protein and 29/46 to nucleocapsid protein; FIGS. 8-9). None of the 15 control subjects detectable antibody responses.

TABLE 1
Convalescent patient demographics (n = 46)

| Description | Value | |
|---|---|---|
| Median age, y (range) | 34.5 | (20-69) |
| Male | 21 | (46%) |
| Disease severity | | |
| Mild | 38 | (83%) |
| Moderate | 3 | (7%) |
| Severe | 1 | (2%) |
| Asymptomatic | 4 | (9%) |
| Symptoms | | |
| Fever | 24 | (52%) |
| Respiratory symptoms | 38 | (83%) |
| Gastrointestinal symptoms | 9 | (20%) |
| Fatigue | 16 | (33%) |
| Anosmia | 20 | (44%) |
| Median length of symptoms, d (range) | 12 | (0-30) |
| Need for hospitalization | 2 | (4%) |

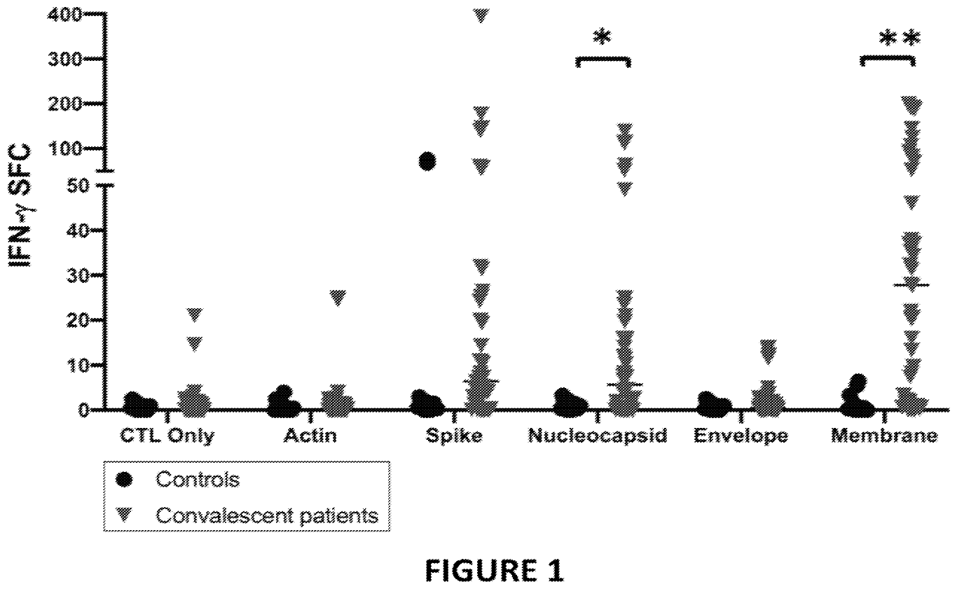

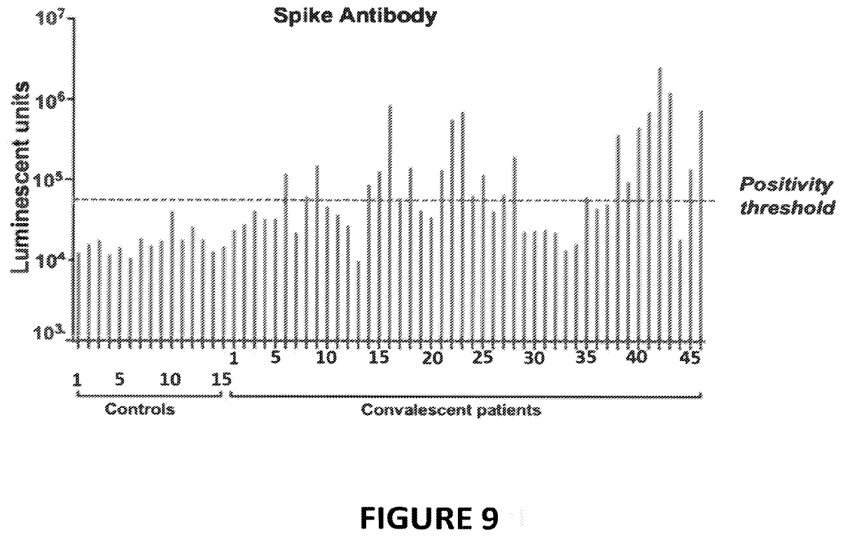

CSTs from convalescent donors are polyfunctional and recognize multiple viral proteins. Following stimulation and expansion of CSTs, specific T-cell activity against SARS-CoV-2 structural proteins were detected in 32 of 46 convalescent donors and 2 of 15 control subjects (FIG. 1) via IFN-γ ELISpot. Convalescent donors predominantly responded to membrane (27/46, P=6.24×10⁻⁶ vs control subjects), followed by spike (12/46, P=0.16 vs control subjects), and nucleocapsid proteins (10/46, P=0.0008 vs control subjects). Nonamplified responses to SARS-CoV-2 viral antigens were detectable from PBMCs via IFN-γ ELISpot in only 2 of 46 patients and 0 of 15 controls (FIG. 10), suggesting that the frequency of the SARS-CoV-2 response is relatively low, consistent with T-cell immune responses observed against other respiratory viruses.

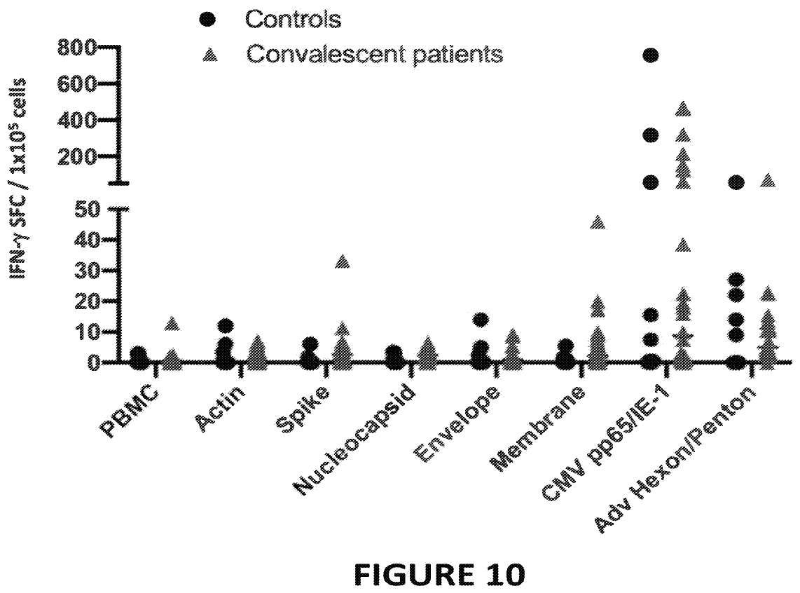

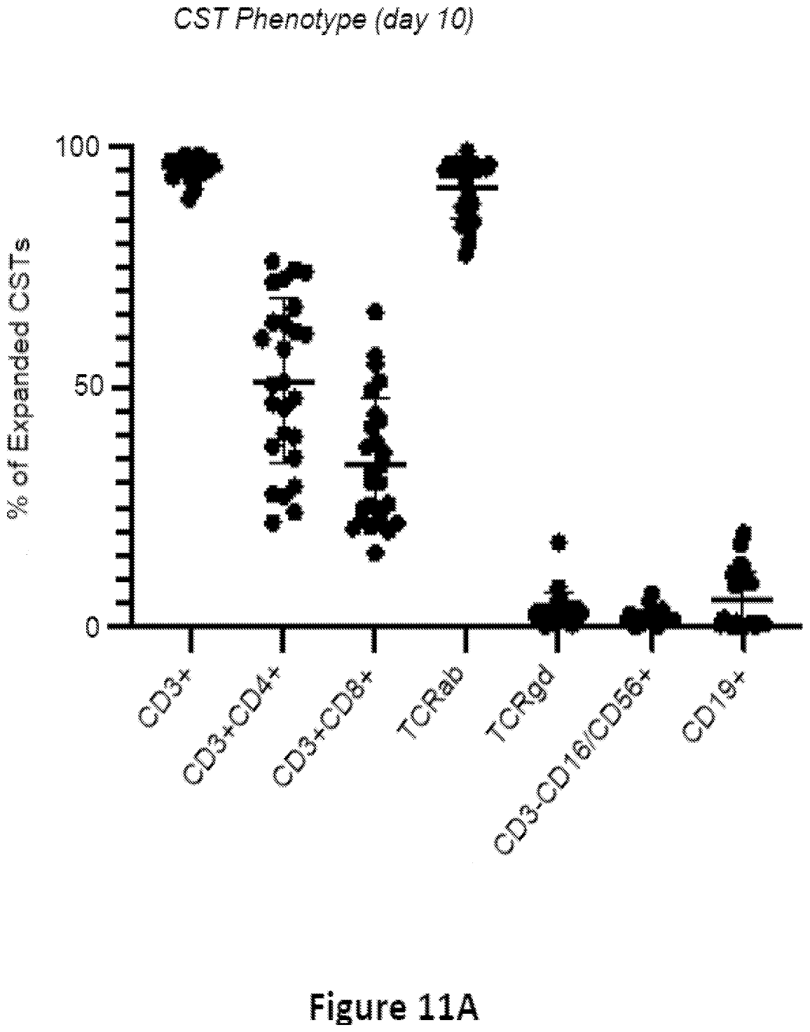

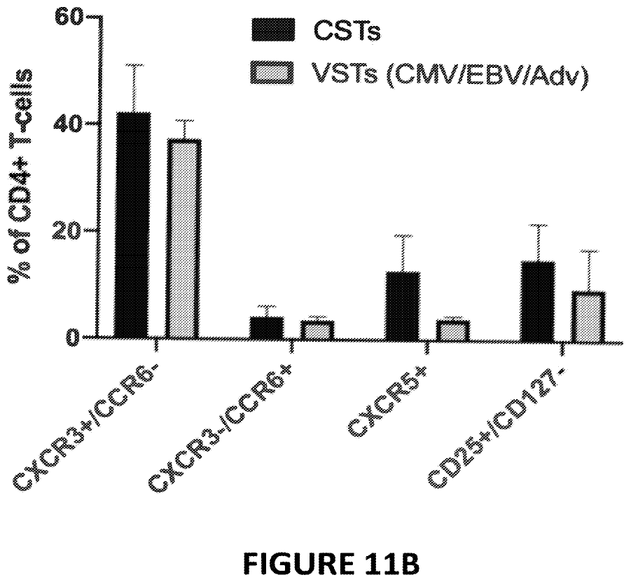

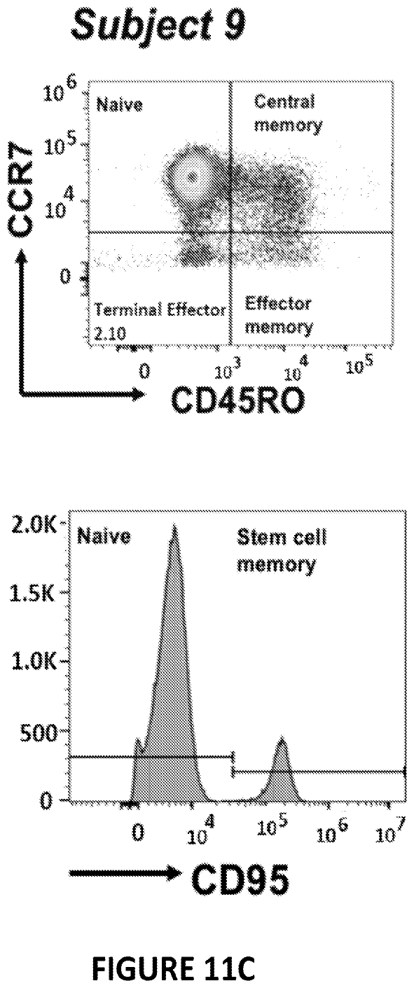

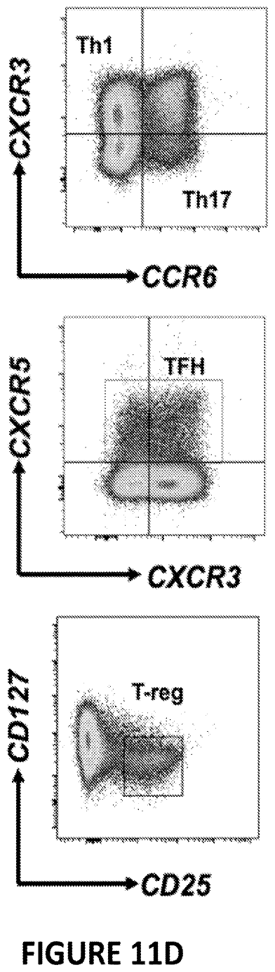

Post-expansion T cells were predominantly CD4⁺, with central memory and effector memory subsets (FIGS. 11A-11D). The predominant CD4⁺ T-cell population was CXCR3⁺CCR6⁻ (mean, 42.3% of CD4+ T cells) consistent with a Th1 population, with minor populations expressing CXCR5⁺/CXCR3⁻ (mean, 12.95% of CD4+ T cells) and CD1272/CD251 (mean, 15.18% of CD4⁺ T cells). These ratios were proportionate to rapidly expanded virus-specific T cells targeting cytomegalovirus, EBV, and adenovirus (FIG. 11B).

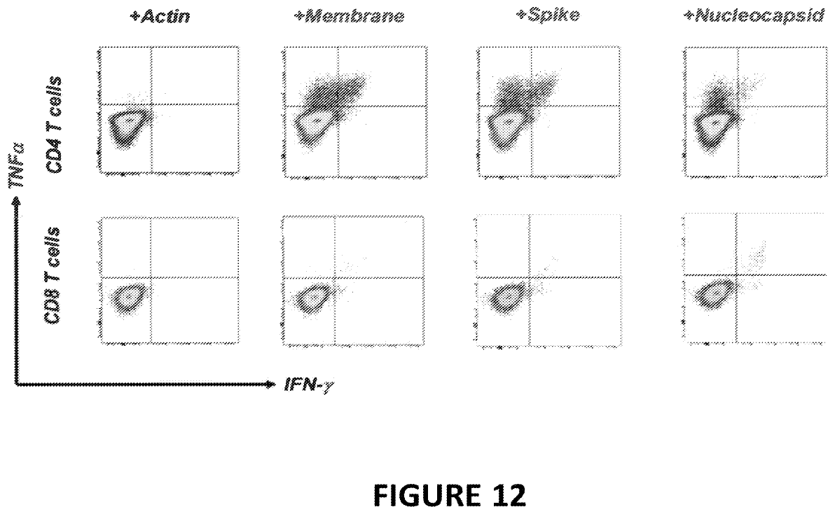

Comparatively, SARS-CoV-2-specific T cells expanded using a similar protocol in 96-well plates rather than the G-Rex10 bioreactor showed somewhat more detectable CD8 reactivity by intracellular staining (FIG. 12), which may suggest that strongly elicited expansion results in preferential outgrowth of the CD4⁺ component.

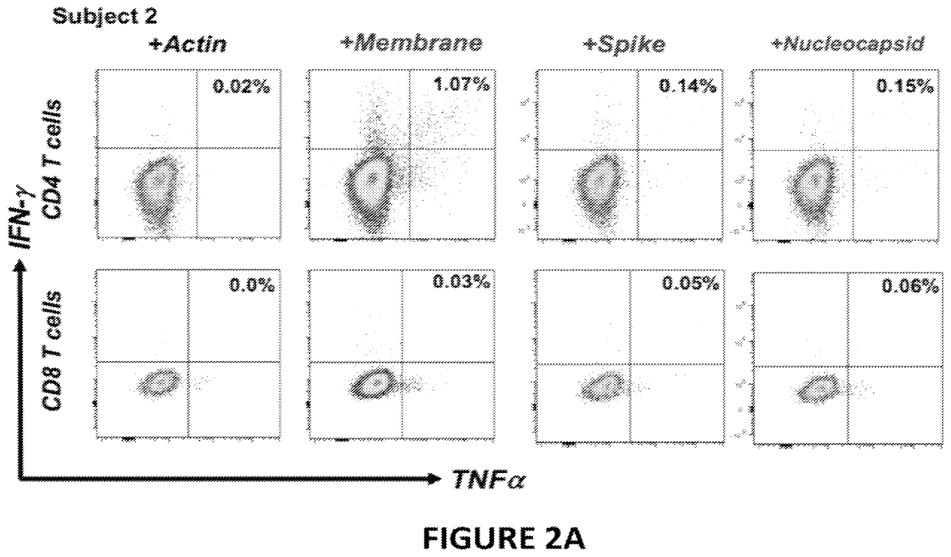

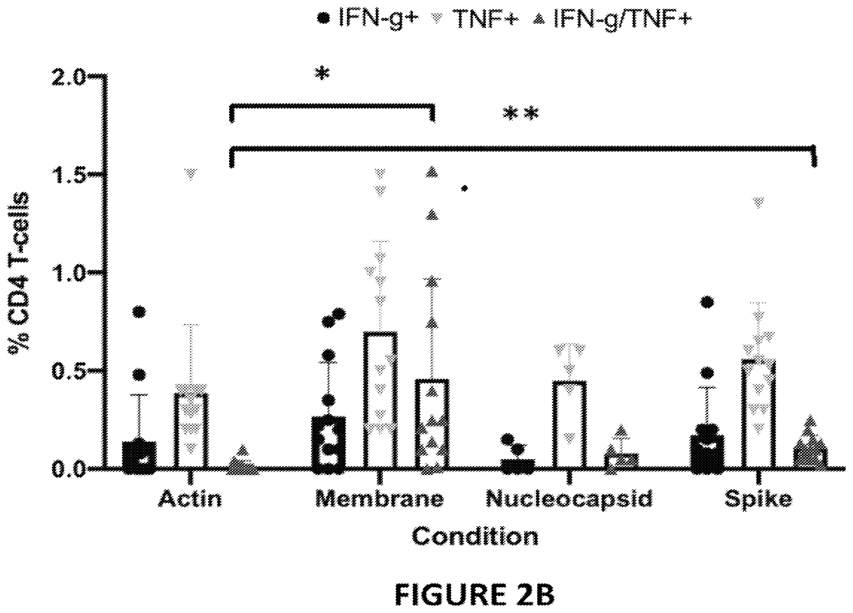

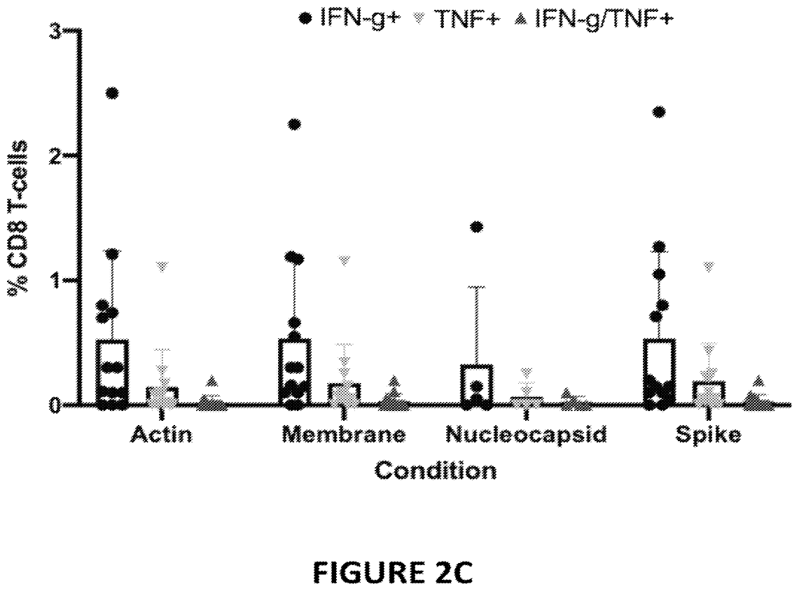

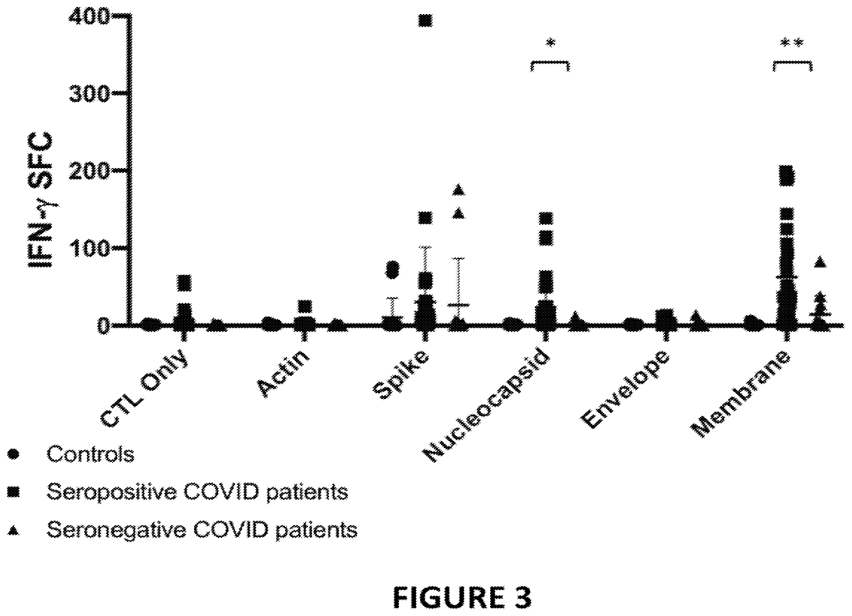

Responses to spike and membrane proteins were confirmed to be predominantly CD4-restricted in 11/11 tested patients (FIG. 2), with significant elevations in IFN-γ/TNF-α-expressing populations targeting membrane and spike proteins (P=0.008 and P=0.0002 in comparison with actin, respectively). Following restimulation with viral structural proteins, CSTs produced multiple cytokines, with significant production of IL-1β, IL-2, IL-4, IL-6, IL-7, IL-12, granulocyte-macrophage colony-stimulating factor, IFN-γ, and TNF-α.

CSTs expanded to 18 days following a second stimulation showed a similar pattern of cytokine production, which was not statistically different from the cytokine profile following the first stimulation, with the exception of lower IFN-γ production in response to spike protein. Alloreactivity testing of CSTs via ⁵¹Cr release assay showed no lysis of HLA-mismatched phytohemagglutinin blasts by T cells following up to 18 days of expansion. Culture of clonal CST populations by limiting dilution and restimulation yielded several CD41 T-cell clones, which showed polyfunctional cytokine production on peptide restimulation.

To assess cross-reactivity, CSTs were tested against peptides corresponding to variant epitopes in circulating SARS-CoV-2 genotypes and from the NL63 and OC43 coronaviruses.31 This testing showed moderate cross-reactivity to described variants in the regions of SARS-CoV-2 epitopes, but minimal cross-reactivity with two homologous nucleocapsid peptides from NL63 and OC43 (FIG. 13).

CSTs from seropositive donors recognize a broader array of viral antigens than CST derived from donors who lack detectable humoral responses. Of the 46 convalescent patients with history of COVID-19, twenty six had demonstrable antibody and T-cell responses to SARS-CoV-2. Seven convalescent donors had no detectable T-cell or antibody responses (FIG. 7). Six donors had antibody responses without detectable T-cell responses and 6 donors had T-cell responses without accompanying antibody responses.

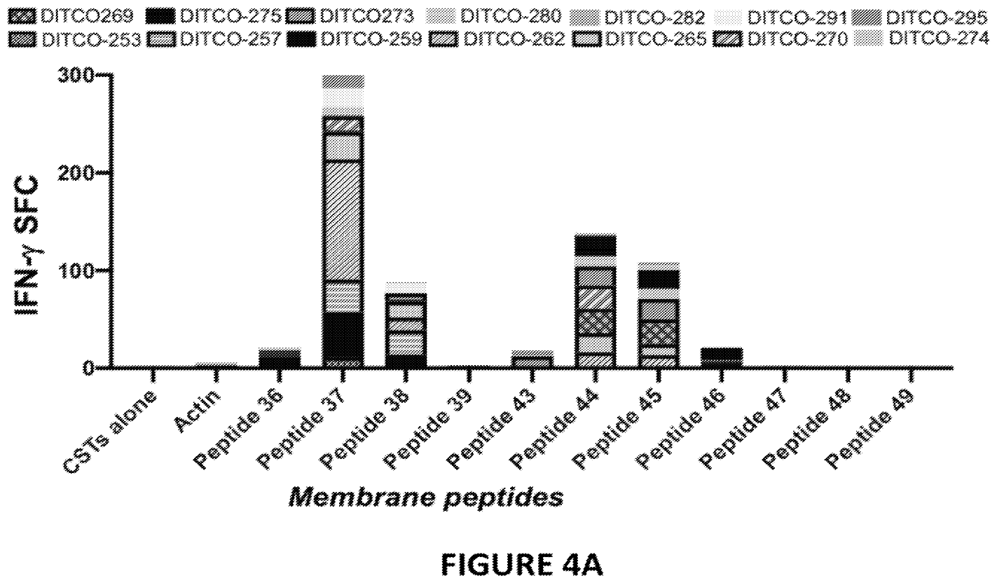

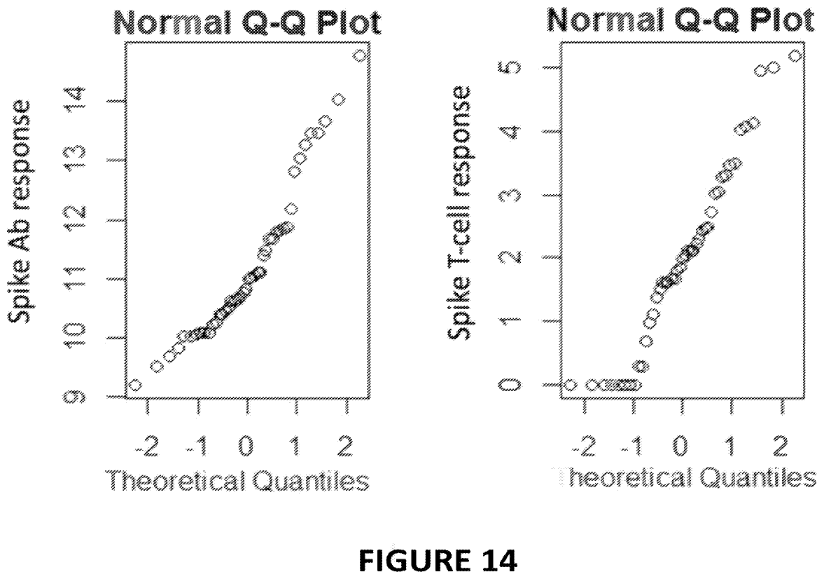

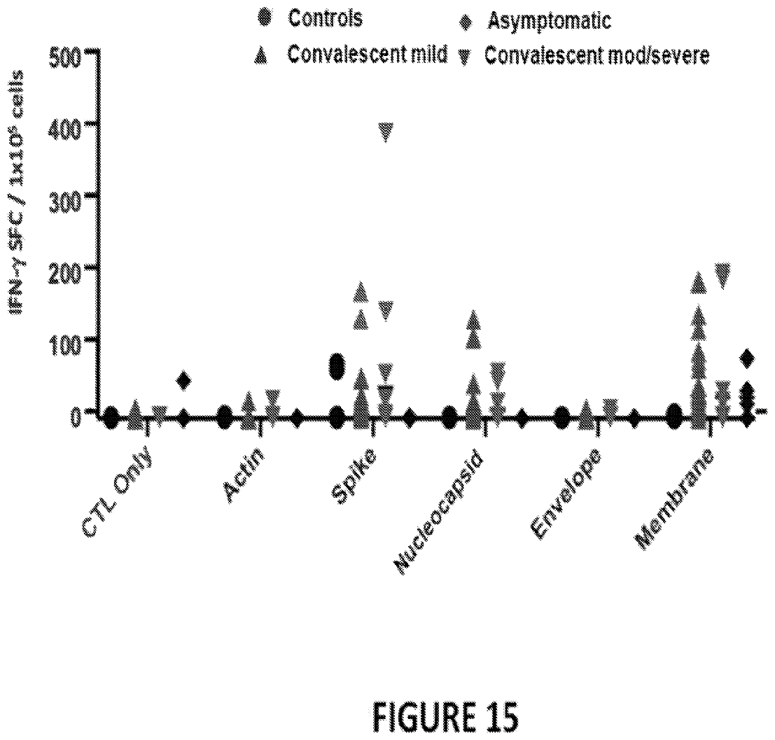

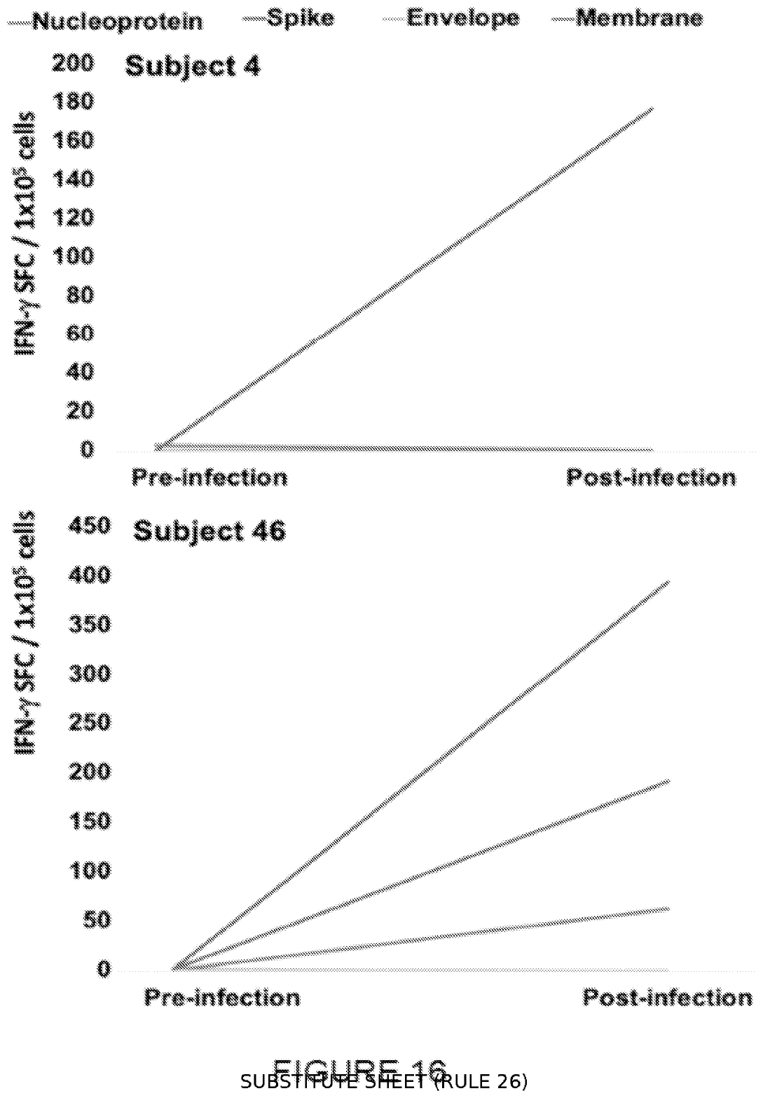

A significant association was noted between presence of an antibody response and T-cell response to spike protein in convalescent patients (P=0.004 via Pearson X² test; FIG. 14). Additionally, seropositive subjects were also more likely to demonstrate a T-cell response to membrane (P=0.00075) and nucleocapsid proteins (P=0.0015) (FIG. 3). Although there was no detectable correlation between disease severity and the magnitude of T-cell or antibody responses (FIG. 15), 14 of the 20 patients who lacked T-cell and/or antibody responses had mild disease, and all 4 asymptomatic donors had incomplete immune responses (3 donors had SARS-CoV-2 T-cell responses only, and 1 donor had detectable SARS-CoV-2 antibody responses only). Evaluation of T-cell responses before COVID-19 infection was able to be performed on 2 subjects who had previously banked cells. Subject 4 had mild gastrointestinal disease, fever, and shortness of breath, and developed a CD41 T-cell response to spike protein (which was not detectable pre-illness), but no detectable antibody response to spike or nucleocapsid. SARS CoV-2 immune (humoral and adaptive) responses were absent in the prepandemic sample, and postinfection (after being confirmed to be PCR⁺ for SARS-CoV-2), a robust T-cell response to spike protein was demonstrated, though this individual did not have an antibody response to spike or nucleocapsid. Subject 46 had mild respiratory symptoms, anosmia, and gastrointestinal symptoms, and developed a T-cell response targeting spike, membrane, and nucleocapsid, as well as antibody response to both spike and nucleocapsid, both of which were absent 2 months before his illness (FIG. 16).

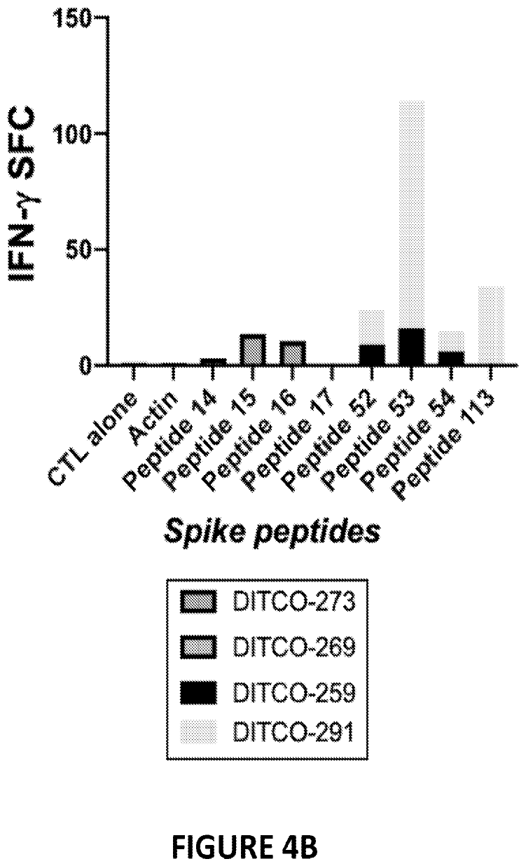

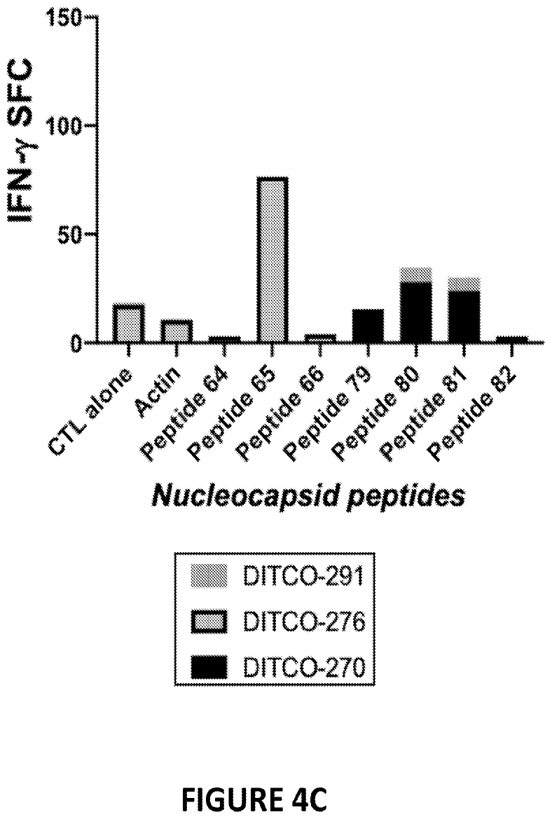

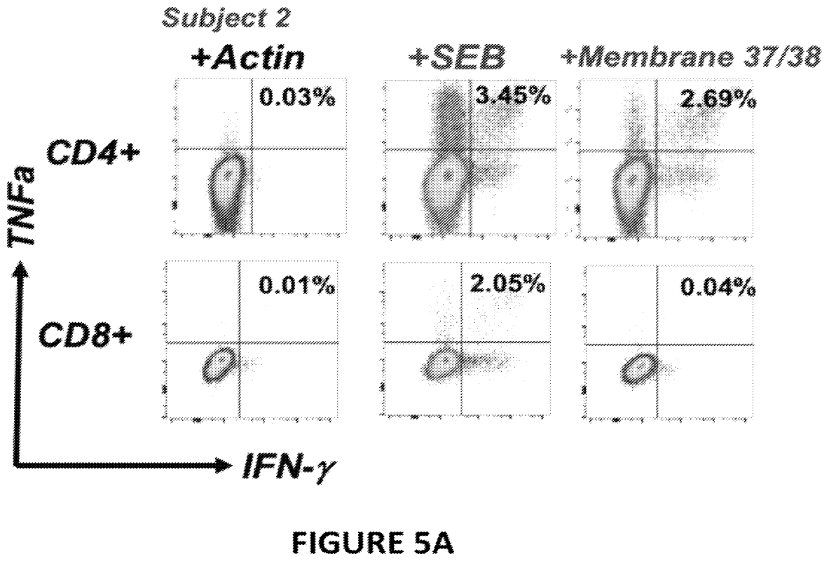

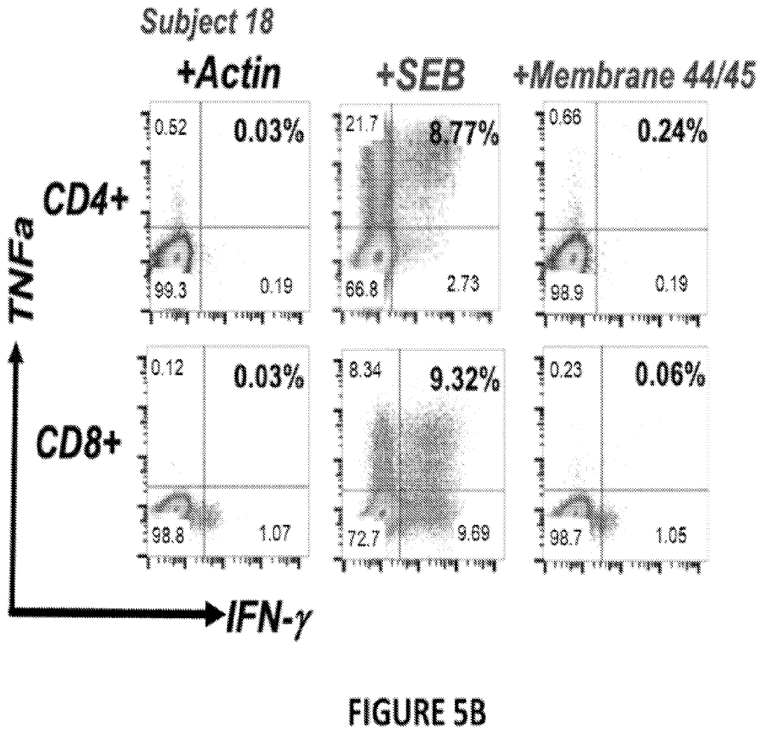

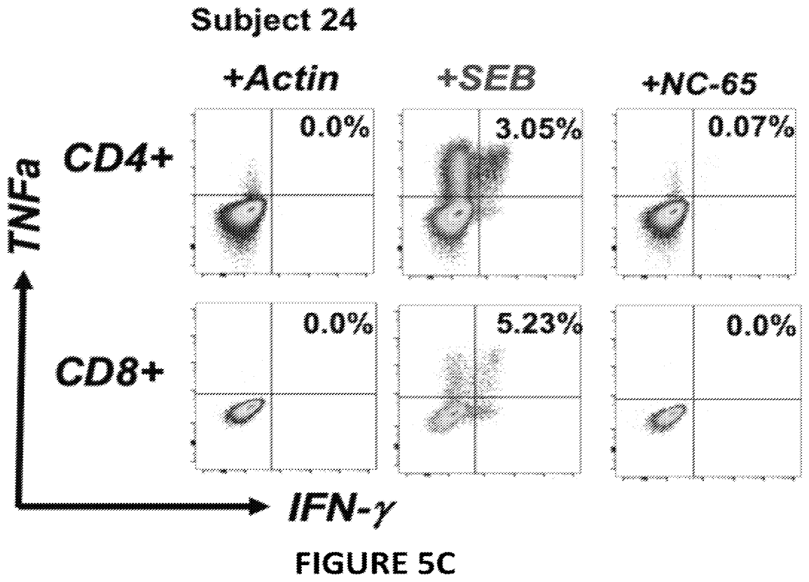

CSTs recognize multiple immunodominant epitopes in membrane, spike, and nucleocapsid proteins. As shown in FIG. 4A, epitope mapping of the membrane protein yielded multiple epitopes at the C-terminal domain. Two epitopes at AA 144-163 were recognized by 8 donors and were exclusively CD4-restricted (FIG. 5A).

Using in silico analysis, the predicted HLA restrictions of these responses were HLA-DRB1*11 and DRB4*01 (Table 2); Reynisson B, et al. Improved prediction of MHC II antigen presentation through integration and motif deconvolution of mass spectrometry MHC eluted ligand data. J Proteome Res. 2020; 19(6):2304-2315. Chen B, et al., Predicting HLA class II antigen presentation through integrated deep learning. NAT BIOTECHNOL. 2019; 37(11):1332-1343.

TABLE 2
Identified class 2 epitopes in membrane, nuclecapsid, and spike proteins and predicted HLA restrictions

| | Amino | | | | | | | | | |
|---|---|---|---|---|---|---|---|---|---|---|
| | acid | | | | | | | | | |
| Peptide sequence | location | Subject | HLA-DRB1 | HLA-DRB3 | HLA-DRB4 | HLA-DRB5 | HLA-DQA1 | HLA-DQB1 | HLA-DPA1 | HLA-DPB1 |
| Membrane | | | | | | | | | | |
| SEQ. ID. NO: 53 | 144-159 | 6 | 07:01, 11:04 | 02:02 | 01:03 | | 02:01, 05:01 | 03:01, 03:03 | 01:03, 02:01 | 03:01, 03:03 |
| LRGHLRIAGHHL | | 10 | 07:01, 11:01 | 02:02 | 01:01 | | 02:01, 05:01 | 02:02, 03:01 | 01:03, 03:01 | 04:01, 11:01 |
| GRC | | 13 | 11:04, 15:02 | 02:02 | 01:01, 01:03 | 01:02 | 01:03, 05:01 | 03:01, 06:01 | 01:03, 02:01 | 02:01, 14:01 |
| | | 18 | 11:04, 15:01 | 02:02 | | | 01:02, 05:01 | 03:01, 06:02 | 01:03, 02:01 | 04:01, 14:01 |
| SEQ. ID NO. 54 | 148-163 | 6 | 07:01, 11:04 | 02:02 | 01:03 | | 02:01, 05:01 | 03:01, 03:03 | 01:03, 02:01 | 03:01, 03:03 |
| LRIAGHHLGRCD | | 13 | 11:04, 15:02 | 02:02 | 01:01, 01:03 | 01:02 | 01:03, 05:01 | 03:01, 06:01 | 01:03, 02:01 | 02:01, 14:01 |
| IKD | | | | | 01:01 | | | | | |
| SEQ. ID NO. 60 | 173-188 | 10 | 07:01, 11:01 | 02:02 | 01:01, 01:03 | 01:02 | 02:01, 05:01 | 02:02, 03:01 | 01:03, 03:01 | 04:01, 11:01 |
| SRTLSYYKLGAS | | 13 | 11:04, 15:02 | 02:02 | 01:01 | 01:01 | 01:03, 05:01 | 03:01, 06:01 | 01:03, 02:01 | 02:01, 14:01 |
| QRV | | 17 | 12:01, 15:01 | 02:02 | | | 01:02, 05:01 | 03:01, 05:01 | 01:03, 01:03 | 02:01, 04:01 |
| | | 18 | 11:04, 15:01 | 02:02 | 01:03, 01:03 | 02:02 | 01:02, 05:01 | 03:01, 06:02 | 01:03, 02:01 | 04:01, 14:01 |
| | | 21 | 03:02, 16:02 | 01:62 | 01:03, 01:03 | | 01:02, 04:01 | 04:02, 05:02 | 02:02, 02:02 | 01:01, 01:01 |
| | | 23 | 01:01, 03:01 | 01:02 | | | 01:01, 05:01 | 02:01, 05:01 | 01:03, 01:03 | 02:01, 02:01 |
| SEQ. ID NO. 61 | 177-192 | 10 | 07:01, 11:02 | 02:02 | 01:01 | | 02:01, 05:01 | 02:02, 03:01 | 01:03, 03:01 | 04:01, 11:01 |
| SYYKLGASQRV | | 17 | 12:01, 15:01 | 02:02 | 01:01 | 01;01 | 01:02, 05:01 | 03:01, 05:01 | 01:03, 01:03 | 02:01, 04:01 |
| AGDS | | 21 | 03:02, 16:02 | 01:62 | 01:03, 01:03 | 02:02 | 01:02, 04:01 | 04:02, 05:02 | 02:02, 02:02 | 01:01, 01:01 |
| | | 23 | 01:01, 03:03 | 01:01 | 01:03, 01:03 | | 01:01, 05:01 | 02:01, 05:01 | 01:03, 01:03 | 02:01, 02:01 |
| SEQ. ID NO. 62 | 181-195 | 23 | 01:01, 03:01 | 01:01 | 01:01. 01:03 | | 01:01, 05:01 | 02:01, 05:01 | 01:03, 01:03 | 02:01, 02:01 |
| LGASQRVAGDS | | | | | | | | | | |
| GFAA | | | | | | | | | | |
| Nucleocapsid | | | | | | | | | | |
| SEQ. ID NO. 450 | 257-271 | 24 | 04:01, 07:01 | 01:01 | 01:03 | | 03:01, 05:05 | 03:01, 03:02 | 02:01, 02:01 | 14:01, 14:01 |
| KPRQKRTATKA | | | | | | | | | | |
| YNVT | | | | | | | | | | |
| SEQ. ID NO. 464 | 313-327 | 18 | 11:04:15:01 | 02:02 | | | 01:02, 05:01 | 03:01, 06:02 | 01:03, 02:01 | 04:01, 14:01 |
| AFFGMSRGMEV | | | | | | | | | | |
| TPS | | | | | | | | | | |
| Spike | | | | | | | | | | |
| SEQ. ID NO. 84 | 57-71 | 8 | 03:01, 13:01 | 01:01 | 01:03 | | 01:03, 05:01 | 02:01, 06:03 | 01:03, 01:03 | 04:01, 04:01 |
| PFFSNVTWFHAI | | | | | | | | | | |
| HVS | | | | | | | | | | |
| SEQ. ID NO. 85 | 61-75 | 8 | 03:01, 13:01 | 01:01 | 01:03 | | 01:03, 05:01 | 02:01, 06:03 | 01:03, 01:03 | 04:01, 04:01 |
| NVTWFHAHVSG | | | | | | | | | | |
| TNG | | | | | | | | | | |
| SEQ. ID NO. 121 | 205-219 | 37 | 03:01. 04:01 | 01:01 | 01:03 | | 03:01, 05:01 | 02:01, 03:02 | 01:03, 01:03 | 02:01, 03:01 |
| SKHTPINLVRDL | | | | | | | | | | |
| PQG | | | | | | | | | | |
| SEQ. ID NO. 122 | 209-223 | 21 | 03:01, 16:02 | 01:62 | 01:01, 01:03 | | 01:02, 04:01 | 04:02, 05:02 | 02:02, 02:02 | 01:01, 01:01 |
| PINLVRDLPQGF | | 37 | 03:01. 04:01 | 01:01 | 01:03 | | 03:01, 05:01 | 02:01, 03:02 | 01:03, 01:03 | 02:01, 03:01 |
| SAL | | | | | | | | | | |
| SEQ. ID NO. 182 | 449-463 | 37 | 03:01, 04:01 | 01:01 | 01:03 | | 03:01, 05:01 | 02:01, 03:02 | 01:03, 01:03 | 02:01, 03:01 |
| YNYLYRLFRKS | | | | | | | | | | |
| NLKP | | | | | | | | | | |

Boldface type indicates a strong binder (<2); italic type indicates a weak binder (2-10).

Similarly, epitopes at AA 173-192 were recognized by 6 donors, and were also confirmed to be CD4-restricted (FIG. 5B). These epitopes lie within the C-terminal domain which is located inside the virion and on intracellular membranes of infected cells that is a conserved region within all known strains of SARS-CoV2; Chang T J, et al. Genomic analysis and comparative multiple sequence of SARS-CoV2. J CHIN MED ASSOC. 2020; 83(6):537-543.

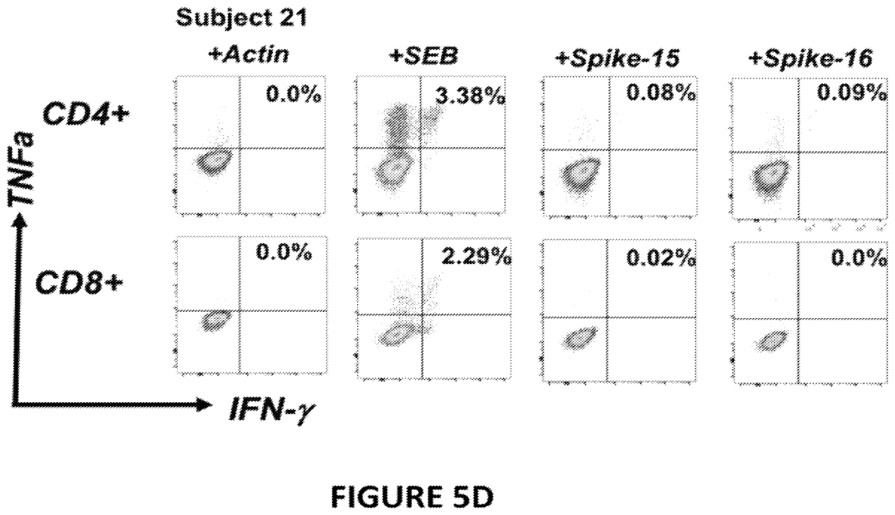

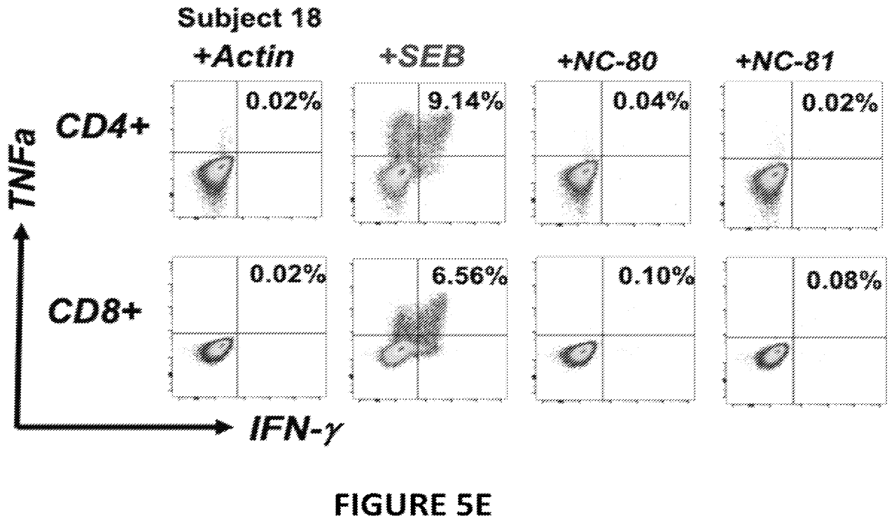

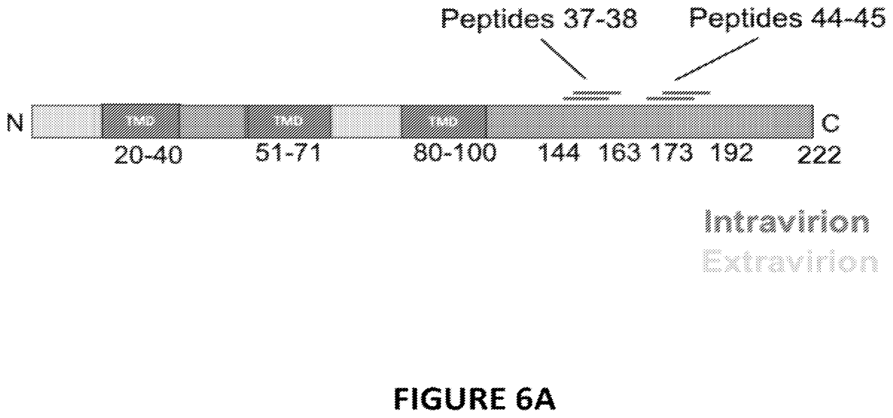

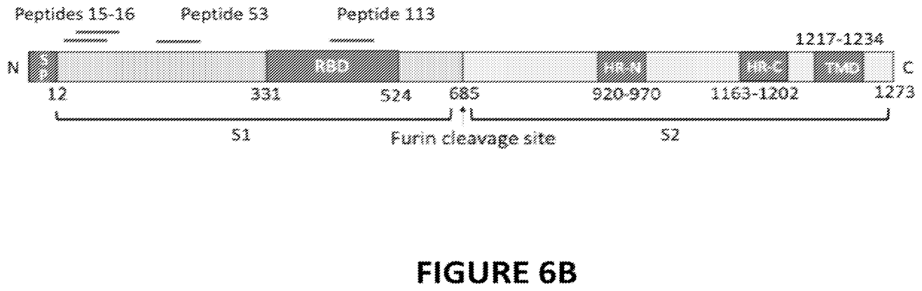

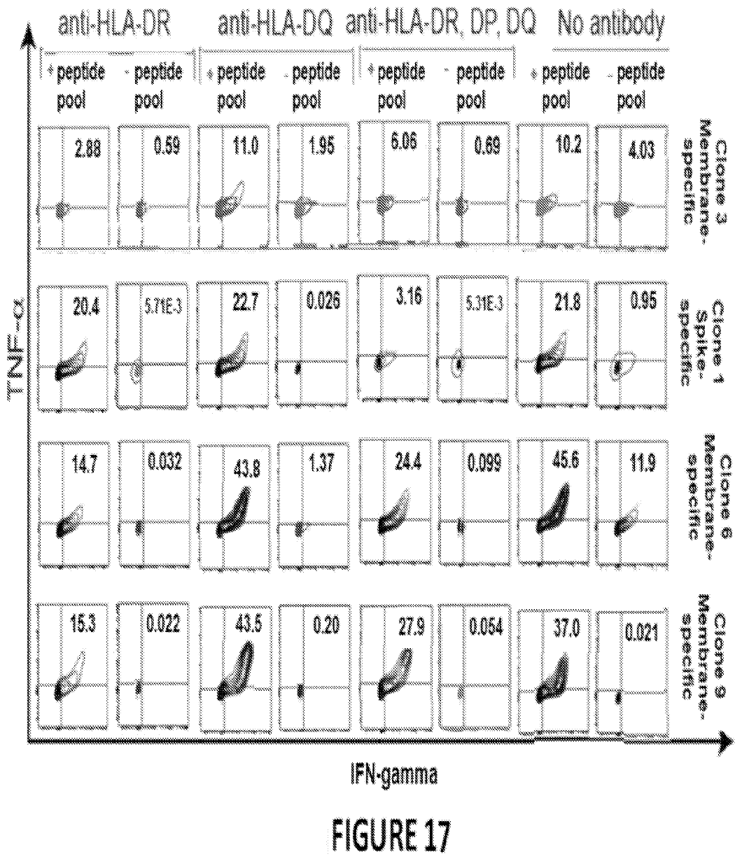

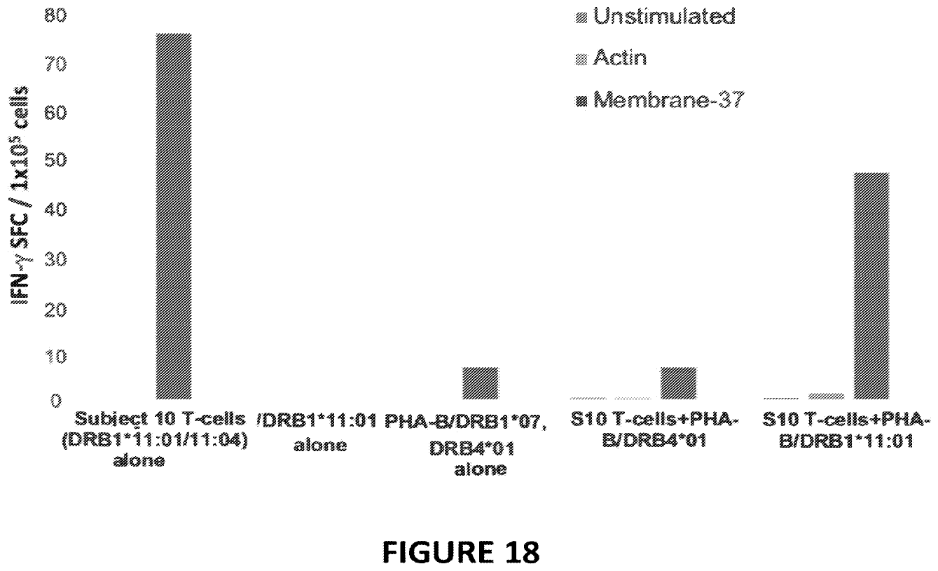

Antibody blocking experiments on clonal SARS-CoV-2 CD41 T cells demonstrated a HLA-DR restriction for several clones (FIG. 17). Confirmatory restriction testing using partially HLA-matched cells confirmed that membrane peptide 37 (AA 145-160) is bound by HLA-DRB1*11:01 (FIG. 18). Epitope mapping of spike protein yielded 3 epitopes (FIG. 4B) within the S1 domain, which were also CD4-restricted (figure within the S1 domain (FIG. 5D). Epitope mapping of nucleocapsid yielded CD4-restricted epitopes at AA 257-271 (FIGS. 4C and 5C), as well as a CD8-restricted epitope at AA 317-335 (FIG. 4C; Table 3). These lie in the dimerization domain and are also highly conserved within SARS-CoV-2 genotypes (FIG. 6); Chang T J, et al. Genomic analysis and comparative multiple sequence of SARS-CoV2. J Chin Med Assoc. 2020; 83(6):537-543; Shen Z, et al. Genomic diversity of SARS-CoV-2 in coronavirus disease 2019patients. CLIN INFECT DIS. 2020; 71(15): 713-720.

TABLE 3
Identified class I epitopes in nucleocapsid and predicted
HLA restrictions

| | Amino acid | | | | |
|---|---|---|---|---|---|
| Peptide sequence | location | Subject | HLA-A | HLA-B | HLA-C |
| SEQ. ID. NO: 465 | 317-331 | 18 | 24:02, 26:01 | 40:01, 44:05* | 02:02, 03:04 |
| MSRIGMEVTPSGTWL | | | | | |
| SEQ. ID. NO: 466 | 321-335 | 18 | 24:02, 26:01 | 40:01, 44:05* | 02:02, 03:04 |
| GMEVTPSGTWLTYTG | | | | | |

Boldface type indicates a strong binder (<5); italic type indicates a weak binder (0.2-5).
*Predicted P*44.05 peptide GMEVTPSGTW.

Advancing knowledge of the immune response to SARS-CoV-2 is critical at the current juncture not only to guide candidate vaccine studies but, importantly, also to identify novel therapeutic targets for the design of a robust therapeutic T-cell product for the treatment of immunocompromised patients with blood disorders. Multiple studies have focused on the antibody response following COVID-19, but the persistence of antibody is unclear.

In immunocompromised patients, including those undergoing BMT, viruses represent a significant risk for morbidity. Though to date, relatively few immunocompromised patients have died of COVID-19 relative to the general population, prolonged illness and prolonged viral shedding has also been described, which could increase risk for other patients and staff. Decker A, et al., Prolonged SARS-CoV-2 shedding and mild course of COVID-19 in a patient after recent heart transplantation [published online 9 Jun. 2020]. AM J TRANSPLANT. doi: 10.1111/ajt.16133; Zhu L, et al. Coronavirus disease 2019 pneumonia in immunosuppressed renal transplant recipients: a summary of 10 confirmed cases in Wuhan, China. EUR UROL. 2020; 77(6):748-754.

Furthermore, even after recovery, this population is likely to be at risk for reinfection because of compromised adaptive responses. Adoptive T-cell immunotherapy may accordingly be beneficial for prevention or early treatment of COVID-19.

As shown herein, the inventors show that ex vivo-expanded CSTs can be easily generated from convalescent patients following recovery from COVID-19, and can recognize multiple immunodominant epitopes within the SARs-CoV-2 membrane protein, which represent class 11 restricted T-cell epitope “hot spots.”

It was demonstrated that SARS-CoV-2 membrane, spike, and nucleocapsid proteins showed a clear hierarchy of immunodominance and were associated with significant increases in IFNγ/TNF-α producing CD4⁺ T-cell populations. Moreover, these results show cross-reactivity with SARS-CoV-2 variant epitopes may permit T-cell responses against these regions to provide protection against circulating viral strains with these mutations.

Further the inventors consider that CSTs derived from a hematopoietic stem cell transplantation donor can be an effective preventive therapy for patients undergoing BMT and that for patients who lack a donor with immunity to COVID-19, the administration of partially HLA matched third-party CSTs may be a consideration as an “on demand” treatment of COVID-19 early in the course of infection to prevent invasive disease with the goal to reduce the length

Nevertheless, the development of a potent “off-the-shelf” virus specific T-cell therapy requires characterization of the T-cell products to discover the epitope specificity and HLA restrictions of the virus specific T cells to ensure optimal matching between the virus-specific T-cell donor and the recipient. As shown herein, multiple regions within the highly conserved C-terminal domain of the membrane protein of SARS-CoV-2 elicited CD4-restricted responses were shared by CST products generated from multiple individuals. The HLA restriction for membrane peptide 37 was confirmed to be mediated by HLADRBI* 11:01, and in silico analysis suggested restriction of additional epitopes through HLA-DR11, DR7, DQ3, and DQ7, which are present in roughly 50% of the population. Klitz W, et al. New HLA haplotype frequency reference standards: high-resolution and large sample typing of HLA DR-DQ haplotypes in a sample of European Americans. TISSUE ANTIGENS. 2003; 62(4):296-307 (HLA haplotype frequencies and other subject matter incorporated by reference).

This information is highly useful for the manufacture of a CST bank for clinical use.

Moreover, given the increased severity of COVID-19 within minority populations, it is important to determine if there are risk associations with specific HLA types, which would need to be accounted for in candidate vaccines and understanding that these HLA restricted responses will be critical for the development of a third-party CST bank to treat the majority of screened high-risk patients (including ethnically diverse populations). Additionally, the demonstration of T-cell responses to described variant epitopes within SARS-CoV-2 suggests that CSTs are likely to have activity against many circulating viral strains in spite of genetic variation. CSTs with specificity for ≥1 viral antigens could be successfully produced from 58% of the evaluated convalescent donors, and an association was detected between SARS-CoV-2 seropositivity and T-cell responses to non-spike antigens.

The absence of these responses in the work above, even following ex vivo expansion, suggests that T-cell reactivity in unexposed individuals is more limited than in seropositive convalescent patients, which may reflect the differences in structural proteins in SARS-CoV-2 vs other commonly circulating coronaviruses (Table 4).

TABLE 4
Epitope homology with other human coronaviruses

| | Other | | |
|---|---|---|---|
| | human | | Amino acid |
| SARS-CoV-2 epitope identified | coronavirus | Protein name | sequence alignment |
| Membrane | SARS | M protein | LRGHLRIAGHHLGRC |
| SEQ. ID. NO: 53 | coronavirus | Membrane | (SEQ ID NO: 493) |
| LRGHLRIAGHHLGRC | HKU1 | glycoprotein | RGHLRMAGHPLGRC |
| | MERS | M protein | (SEQ ID NO: 494) |
| | | | RGHLYIQGVKLG |
| | | | (SEQ ID NO: 495) |
| | | | GHLKIAGMHFGAC |
| | | | (SEQ ID NO: 496) |
| SEQ. ID. NO: 54 | SARS | Membrane | LRIAGHHLGRCDIKD |
| LRIAGHHLGRCDIKD | coronavirus | protein | (SEQ ID NO: 497) |
| | | | LRMAGHPLGRCDIKD |
| | | | (SEQ ID NO: 498) |
| SEQ. ID. NO: 60 | SARS | Membrane | SRTLSYYKLGASQRV |
| SRTLSYYKLGASQRV | coronavirus | protein | (SEQ ID NO: 499) |
| | | | SRTLSYYKLGASQRV |
| | | | (SEQ ID NO: 500) |
| SEQ. ID. NO: 61 | SARS | Membrane | SYYKLGASQRVAGDS |
| SYYKLGASQRVAGDS | coronavirus | protein | (SEQ ID NO: 501) |
| | | | SYYKLGASQRVGTDS |
| | | | (SEQ ID NO: 502) |
| SEQ. ID. NO: 62 | SARS | Membrane | LGASQRVAGDSGFAA |
| LGASORVAGDSGFAA | coronavirus | glycoprotein | (SEQ ID NO: 503) |
| | NL63 | Orf1a protein | LGASQRVGTDSGFAA |
| | | | (SEQ ID NO: 504) |
| | | | LGAS-VTEDVKFAA |
| | | | (SEQ ID NO: 505) |
| Nucleocapsid | | | |
| SEQ. ID. NO: 450 | SARS | Nucleocapsid | KPRQKRTATKAYNVT |
| KPRQKRTATKAYNVT | coronavirus | protein | (SEQ ID NO: 506) |
| | OC43 | Nucleocapsid | KPRQKRTATKQYNVT |
| | NL63 | protein | (SEQ ID NO: 507) |
| | MERS | Chain A, | KPRQKRSPNK |
| | | nucleocapsid | (SEQ ID NO: 508) |
| | | Nucleocapsid | KPRWKRVPTREENV |
| | | protein | (SEQ ID NO: 509) |
| | | | RHKRVATKSFNV |
| | | | (SEQ ID NO: 510) |
| SEQ. ID. NO: 464 | SARS | Nucleocapsid | AFFGMSRIGMEVTPS |
| AFFGMSRIGMEVTPS | coronavirus | protein N | (SEQ ID NO: 511) |
| | | | AFFGMSRIGMEVTPS |
| | | | (SEQ ID NO: 512) |
| SEQ. ID. NO: 465 | SARS | Nucleocapsid | MSRIGMEVTPSGTWL |
| MSRIGMEVTPSGTWL | coronavirus | protein | (SEQ ID NO: 513) |
| | | | MSRIGMEVTPSGTWL |
| | | | (SEQ ID NO: 514) |
| SEQ. ID. NO: 466 | SARS | Nucleocapsid | GMEVTPSGTWLTYTG |
| GMEVTPSGTWLTYTG | coronavirus | protein | (SEQ ID NO: 515) |
| | | | GMEVTPSGTWLTY |
| | | | (SEQ ID NO: 516) |
| Spike | | | |
| SEQ. ID. NO: 84 | — | — | — |
| PFFSNVTWFHAIHVS | | | |
| SEQ. ID. NO: 85 | — | — | — |
| NVTWFAIHVSGTNG | | | |
| SEQ. ID. NO: 121 | OC43 | Replicase | SKHTPINLVRDLPQFG |
| SKHTPINLVRDLPQG | SARS | polyprotein | (SEQ ID NO: 517) |
| | coronavirus | lab | PANIV-LPQG |
| | | S1 protein | (SEQ ID NO: 518) |
| | | Chain A, | PIDVVRDLPSG |
| | | spike | (SEQ ID NO: 519) |
| | | Replicase | PINLVRDLPQGFSAL |
| SEQ. ID. NO: 122 | SARS | polyprotein | (SEQ ID NO: 520) |
| PINLVRDLPQGFSAL | coronavirus | lab | PIDVVRDLPSGFNTL |
| | OC43 | | (SEQ ID NO: 521) |
| | | | PANIV-LPQG |
| | | | (SEQ ID NO: 522) |
| SEQ. ID. NO: 182 | SARS | Chain E, | YNYLYRLFRKSNLKP |
| YNYLYRLFRKSNLKP | coronavirus | spike | (SEQ ID NO: 523) |
| | | glycoprotein | YNYKYRYLRHGKLRP |
| | | | (SEQ ID NO: 524) |

Boldface type indicates a strong binder (>2).
MERS, Middle East respiratory syndrome.

The data shown herein suggest that using donors with confirmed humoral immunity to SARS-CoV-2 will enable the generation of broadly antigen- and epitope-specific therapeutic T-cell products. In some embodiments, seropositivity or T cell specificity of a donor may be evaluated against one or more peptide epitopes described by Table 4, including SARS-CoV-2 epitopes and coronavirus epitopes.

As shown, the vast majority of the convalescent donors had uncomplicated disease. The data herein suggest that T-cell and humoral responses measured here represent an effective adaptive immune response to SARS-CoV-2 that can be effectively harnessed (especially from BMT donors) for the manufacture of CST products for clinical use. Moreover, all of the evaluated patients survived and recovered without significant inflammatory or thrombotic complications which is consistent with the detected T-cell responses representing beneficial adaptive cellular responses.

As shown herein, a broadly specific T-cell therapeutic targeting three structural proteins of SARS-CoV-2 can be reliably expanded using GMP-compliant methodologies from the majority of convalescent donors. The CST products are principally comprised CD4⁺ T cells specific for conserved regions of these proteins and most frequently the membrane protein.

The immunodominance of the membrane protein has important implications for vaccine development to elicit cellular immune responses because most current vaccine candidates are focused exclusively on the spike protein to elicit neutralizing antibody. However, the disclosure above enables the rapid translation of this novel treatment to the clinic.

Terminology. Terminology used herein is for the purpose of describing particular embodiments only and is not intended to be limiting of the invention.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology. As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1. A method for treating a subject infected with, or at risk of infection by, SARS-COV-2, comprising:
administering to a subject in need thereof ex vivo primed or expanded SARS-CoV-2 antigen-specific T cells that recognize at least one peptide antigen consisting of Peptide 37 (SEQ ID NO: 53), Peptide 44 (SEQ ID NO: 60), Peptide 45 (SEQ ID NO: 61), Peptide 38 (SEQ ID NO: 54), or any one of the peptide antigens described by SEQ ID NOS: 1-524,
wherein said ex vivo primed or expanded SARS-CoV-2 antigen-specific cells are derived from cells of a donor previously infected by SARS-CoV-2 or who has been immunized with SARS-CoV-2 antigen(s), whose antibody levels to one or more SARS-CoV-2 antigens are greater than a control value from an uninfected or unvaccinated subject; or, alternatively,
wherein said ex vivo primed or expanded SARS-CoV-2 antigen-specific cells are derived cells of a donor whose antibody levels to one or more SARS-CoV-2 antigens are no more than a control value from an uninfected or unvaccinated subject.

2. The method of claim 1, wherein said ex vivo primed or expanded SARS-CoV-2 antigen-specific cells are derived from cells of a donor previously infected by SARS-CoV-2 or immunized with SARS-CoV-2 antigen(s), whose antibody levels to one or more SARS-CoV-2 antigens are greater than a control value from an uninfected or unvaccinated subject.

3. The method of claim 1, wherein said ex vivo primed or expanded SARS-CoV-2 antigen-specific cells are derived cells of a donor whose antibody levels to one or more SARS-CoV-2 antigen(s) are no more than a control value from an uninfected or unvaccinated subject.

4.-16. (canceled)

17. The method of claim 1, wherein said SARS-CoV-2 antigen-specific T cells are autologous or fully histocompatible to the subject.

18. (canceled)

19. The method of claim 1, where SARS-CoV-2 antigen-specific T cells are non-autologous and share at least one major histocompatibility antigen with the subject.

20.-28. (canceled)

29. A method for producing SARS-COV-2 antigen-specific T cells comprising:
contacting donor PBMCs or hematopoietic cells with one or more peptides or peptide antigens described by SEQ ID NOS: 1-524 or with a peptide library or peptide libraries spanning one or more SARS-CoV-2 antigens,
culturing the resulting PBMCs or hematopoietic cells with IL-4 and IL-7, and
isolating T cells which recognize one or more SARS-CoV-2 antigens.

30. The method of claim 29, further comprising restimulating the cultured or isolated T cells which recognize SARS-CoV-2 antigen(s) in the presence of irradiated antigen presenting cells loaded with the one or more peptides or peptide antigens described by SEQ ID NOS: 1-524 or with a peptide library or peptide libraries spanning one or more SARS-CoV-2 antigens and then culturing in the presence of IL-4 and IL-7.

31. The method of claim 29, further comprising separating antigen-specific T cells into subpopulation(s) enriched for CD4⁺ T cells, CD8⁺ T cells, or CD44 (high) cells.

32. The method of claim 29, further comprising administering said SARS-COV-2 antigen-specific T-cells to a subject in need thereof.

33. The method of claim 29, wherein said donor has antibody levels to one or more SARS-CoV-2 antigens which are greater than a control value from subject(s) uninfected or by SARS-CoV-2.

34. The method of claim 29, wherein said donor has antibody levels to one or more SARS-CoV-2 antigens which are no more than a control value from subject(s) uninfected or for SARS-CoV-2.

35. The method of claim 29, wherein said donor is or has convalesced from SARS-CoV-2 infection.

36. The method of claim 29, wherein said donor has been immunized to at least one SARS-CoV-2 antigen.

37. The method of claim 29, wherein said donor has not been previously exposed to SARS-CoV-2 or said donor cells are naïve to one or more SARS-CoV-2 antigens.

38. A method for producing SARS-COV-2 antigen-specific T cells comprising:
(a) dividing mononuclear cells from a donor into two portions;
(b) contacting a first portion of said sample with PHA or another mitogen and, optionally with IL-2, to produce ATCs (“activated T cells”) and treating the ATCs with radiation or another agent to inhibit their outgrowth;
(c) separating non-adherent or CD3⁺ T-cells and T-cell precursor cells from adherent cells, CD11C⁺, or CD14 dendritic cells and dendritic precursor cells;
(d) cryopreserving or otherwise reserving the non-adherent or CD3⁺ cells,
(e) contacting the adherent, CD11C⁺, or CD14 cells in the second portion with IL-4 and GM-CSF or other cytokine(s) and/or other agent(s) that generate and mature dendritic cell and with at least one SARS-COV-2 peptide antigen of SEQ ID NOS: 1-524 or a SARS-CoV-2 peptide library to produce antigen-presenting dendritic cells that present the at least one peptide antigen, and treating said antigen-presenting dendritic cells with radiation or another agent sufficient to inhibit their outgrowth;
(f) contacting the reserved non-adherent cells from (d) with the dendritic antigen presenting cells produced in (e) in the presence of IL-7 and IL-15 and optionally other cytokines, to produce virus- or other antigen-specific T-cells that recognize the at least one peptide antigen;
(g) contacting SARS-COV-2 antigen-specific T-cells produced by (f) with the ATCs of (b) in the presence of the at least one peptide antigen in the presence of K562 cells or other accessory cells and in the presence of IL-15; optionally, repeating (g) one or more times;
(h) recovering antigen-specific T-cells that recognize the at least one SARS-COV-2 peptide antigen.

39. The method of claim 38, further comprising administering said SARS-COV-2 antigen-specific T-cells to a subject in need thereof.

40. The method of claim 38, further comprising separating antigen-specific T cells into subpopulations enriched for CD4⁺ T cells, CD8⁺ T cells, or CD44 (high) cells.

41. The method of claim 38, wherein said donor has antibody levels to one or more SARS-CoV-2 antigens which are greater than a control value from subject(s) uninfected by SARS-CoV-2 or from unvaccinated subjects.

42. The method of claim 38, wherein said donor has antibody levels to one or more SARS-CoV-2 antigens that are no more than a control value from subject(s) uninfected by SARS-CoV-2 or from unvaccinated subjects.

43. The method of claim 38, wherein said donor is convalescing or has convalesced from SARS-CoV-2 infection or has been immunized to at least one SARS-CoV-2 antigen.

44.-90. (canceled)


SEQUENCE LISTING (524 sequences)

SEQ ID NO: 1 - protein, 15 aa, Severe acute respiratory syndrome coronavirus 2
MYSFVSEETGTLIVN

SEQ ID NO: 2 - protein, 15 aa, Severe acute respiratory syndrome coronavirus 2
VSEETGTLIVNSVLL

SEQ ID NO: 3 - protein, 15 aa, Severe acute respiratory syndrome coronavirus 2
TGTLIVNSVLLFLAF

SEQ ID NO: 4 - protein, 15 aa, Severe acute respiratory syndrome coronavirus 2
IVNSVLLFLAFVVFL

SEQ ID NO: 5 - protein, 15 aa, Severe acute respiratory syndrome coronavirus 2
VLLFLAFVVFLLVTL

SEQ ID NO: 6 - protein, 15 aa, Severe acute respiratory syndrome coronavirus 2
LAFVVFLLVTLAILT

SEQ ID NO: 7 - protein, 15 aa, Severe acute respiratory syndrome coronavirus 2
VFLLVTLAILTALRL

SEQ ID NO: 8 - protein, 15 aa, Severe acute respiratory syndrome coronavirus 2
VTLAILTALRLCAYC

SEQ ID NO: 9 - protein, 15 aa, Severe acute respiratory syndrome coronavirus 2
ILTALRLCAYCCNIV

SEQ ID NO: 10 - protein, 15 aa, Severe acute respiratory syndrome coronavirus 2
LRLCAYCCNIVNVSL

SEQ ID NO: 11 - protein, 15 aa, Severe acute respiratory syndrome coronavirus 2
AYCCNIVNVSLVKPS

SEQ ID NO: 12 - protein, 15 aa, Severe acute respiratory syndrome coronavirus 2
NIVNVSLVKPSFYVY

SEQ ID NO: 13 - protein, 15 aa, Severe acute respiratory syndrome coronavirus 2
VSLVKPSFYVYSRVK

SEQ ID NO: 14 - protein, 15 aa, Severe acute respiratory syndrome coronavirus 2
KPSFYVYSRVKNLNS

SEQ ID NO: 15 - protein, 15 aa, Severe acute respiratory syndrome coronavirus 2
YVYSRVKNLNSSRVP

SEQ ID NO: 16 - protein, 15 aa, Severe acute respiratory syndrome coronavirus 2
RVKNLNSSRVPDLLV

SEQ ID NO: 17 - protein, 15 aa, Severe acute respiratory syndrome coronavirus 2
MADSNGTITVEELKK

SEQ ID NO: 18 - protein, 15 aa, Severe acute respiratory syndrome coronavirus 2
NGTITVEELKKLLEQ

SEQ ID NO: 19 - protein, 15 aa, Severe acute respiratory syndrome coronavirus 2
TVEELKKLLEQWNLV

SEQ ID NO: 20 - protein, 15 aa, Severe acute respiratory syndrome coronavirus 2
LKKLLEQWNLVIGFL

SEQ ID NO: 21 - protein, 15 aa, Severe acute respiratory syndrome coronavirus 2
LEQWNLVIGFLFLTW

SEQ ID NO: 22 - protein, 15 aa, Severe acute respiratory syndrome coronavirus 2
NLVIGFLFLTWICLL

SEQ ID NO: 23 - protein, 15 aa, Severe acute respiratory syndrome coronavirus 2
GFLFLTWICLLQFAY

SEQ ID NO: 24 - protein, 15 aa, Severe acute respiratory syndrome coronavirus 2
LTWICLLQFAYANRN

SEQ ID NO: 25 - protein, 15 aa, Severe acute respiratory syndrome coronavirus 2
CLLQFAYANRNRFLY

SEQ ID NO: 26 - protein, 15 aa, Severe acute respiratory syndrome coronavirus 2
FAYANRNRFLYIIKL

SEQ ID NO: 27 - protein, 15 aa, Severe acute respiratory syndrome coronavirus 2
NRNRFLYIIKLIFLW

SEQ ID NO: 28 - protein, 15 aa, Severe acute respiratory syndrome coronavirus 2
FLYIIKLIFLWLLWP

SEQ ID NO: 29 - protein, 15 aa, Severe acute respiratory syndrome coronavirus 2
IKLIFLWLLWPVTLA

SEQ ID NO: 30 - protein, 15 aa, Severe acute respiratory syndrome coronavirus 2
FLWLLWPVTLACFVL

SEQ ID NO: 31 - protein, 15 aa, Severe acute respiratory syndrome coronavirus 2
LWPVTLACFVLAAVY

SEQ ID NO: 32 - protein, 15 aa, Severe acute respiratory syndrome coronavirus 2
TLACFVLAAVYRINW

SEQ ID NO: 33 - protein, 15 aa, Severe acute respiratory syndrome coronavirus 2
FVLAAVYRINWITGG

SEQ ID NO: 34 - protein, 15 aa, Severe acute respiratory syndrome coronavirus 2
AVYRINWITGGIAIA

SEQ ID NO: 35 - protein, 15 aa, Severe acute respiratory syndrome coronavirus 2
INWITGGIAIAMACL

SEQ ID NO: 36 - protein, 15 aa, Severe acute respiratory syndrome coronavirus 2
TGGIAIAMACLVGLM

SEQ ID NO: 37 - protein, 15 aa, Severe acute respiratory syndrome coronavirus 2
AIAMACLVGLMWLSY

SEQ ID NO: 38 - protein, 15 aa, Severe acute respiratory syndrome coronavirus 2
ACLVGLMWLSYFIAS

SEQ ID NO: 39 - protein, 15 aa, Severe acute respiratory syndrome coronavirus 2
GLMWLSYFIASFRLF

SEQ ID NO: 40 - protein, 15 aa, Severe acute respiratory syndrome coronavirus 2
LSYFIASFRLFARTR

SEQ ID NO: 41 - protein, 15 aa, Severe acute respiratory syndrome coronavirus 2
IASFRLFARTRSMWS

SEQ ID NO: 42 - protein, 15 aa, Severe acute respiratory syndrome coronavirus 2
RLFARTRSMWSFNPE

SEQ ID NO: 43 - protein, 15 aa, Severe acute respiratory syndrome coronavirus 2
RTRSMWSFNPETNIL

SEQ ID NO: 44 - protein, 15 aa, Severe acute respiratory syndrome coronavirus 2
MWSFNPETNILLNVP

SEQ ID NO: 45 - protein, 15 aa, Severe acute respiratory syndrome coronavirus 2
NPETNILLNVPLHGT

SEQ ID NO: 46 - protein, 15 aa, Severe acute respiratory syndrome coronavirus 2
NILLNVPLHGTILTR

SEQ ID NO: 47 - protein, 15 aa, Severe acute respiratory syndrome coronavirus 2
NVPLHGTILTRPLLE

SEQ ID NO: 48 - protein, 15 aa, Severe acute respiratory syndrome coronavirus 2
HGTILTRPLLESELV

SEQ ID NO: 49 - protein, 15 aa, Severe acute respiratory syndrome coronavirus 2
LTRPLLESELVIGAV

SEQ ID NO: 50 - protein, 15 aa, Severe acute respiratory syndrome coronavirus 2
LLESELVIGAVILRG

SEQ ID NO: 51 - protein, 15 aa, Severe acute respiratory syndrome coronavirus 2
ELVIGAVILRGHLRI

SEQ ID NO: 52 - protein, 15 aa, Severe acute respiratory syndrome coronavirus 2
GAVILRGHLRIAGHH

SEQ ID NO: 53 - protein, 15 aa, Severe acute respiratory syndrome coronavirus 2
LRGHLRIAGHHLGRC

SEQ ID NO: 54 - protein, 15 aa, Severe acute respiratory syndrome coronavirus 2
LRIAGHHLGRCDIKD

SEQ ID NO: 55 - protein, 15 aa, Severe acute respiratory syndrome coronavirus 2
GHHLGRCDIKDLPKE

SEQ ID NO: 56 - protein, 15 aa, Severe acute respiratory syndrome coronavirus 2
GRCDIKDLPKEITVA

SEQ ID NO: 57 - protein, 15 aa, Severe acute respiratory syndrome coronavirus 2
IKDLPKEITVATSRT

SEQ ID NO: 58 - protein, 15 aa, Severe acute respiratory syndrome coronavirus 2
PKEITVATSRTLSYY

SEQ ID NO: 59 - protein, 15 aa, Severe acute respiratory syndrome coronavirus 2
TVATSRTLSYYKLGA

SEQ ID NO: 60 - protein, 15 aa, Severe acute respiratory syndrome coronavirus 2
SRTLSYYKLGASQRV

SEQ ID NO: 61 - protein, 15 aa, Severe acute respiratory syndrome coronavirus 2
SYYKLGASQRVAGDS

SEQ ID NO: 62 - protein, 15 aa, Severe acute respiratory syndrome coronavirus 2
LGASQRVAGDSGFAA

SEQ ID NO: 63 - protein, 15 aa, Severe acute respiratory syndrome coronavirus 2
QRVAGDSGFAAYSRY

SEQ ID NO: 64 - protein, 15 aa, Severe acute respiratory syndrome coronavirus 2
GDSGFAAYSRYRIGN

SEQ ID NO: 65 - protein, 15 aa, Severe acute respiratory syndrome coronavirus 2
FAAYSRYRIGNYKLN

SEQ ID NO: 66 - protein, 15 aa, Severe acute respiratory syndrome coronavirus 2
SRYRIGNYKLNTDHS

SEQ ID NO: 67 - protein, 15 aa, Severe acute respiratory syndrome coronavirus 2
IGNYKLNTDHSSSSD

SEQ ID NO: 68 - protein, 15 aa, Severe acute respiratory syndrome coronavirus 2
KLNTDHSSSSDNIAL

SEQ ID NO: 69 - protein, 14 aa, Severe acute respiratory syndrome coronavirus 2
DHSSSSDNIALLVQ

SEQ ID NO: 70 - protein, 15 aa, Severe acute respiratory syndrome coronavirus 2
MFVFLVLLPLVSSQC

SEQ ID NO: 71 - protein, 15 aa, Severe acute respiratory syndrome coronavirus 2
LVLLPLVSSQCVNLT

SEQ ID NO: 72 - protein, 15 aa, Severe acute respiratory syndrome coronavirus 2
PLVSSQCVNLTTRTQ

SEQ ID NO: 73 - protein, 15 aa, Severe acute respiratory syndrome coronavirus 2
SQCVNLTTRTQLPPA

SEQ ID NO: 74 - protein, 15 aa, Severe acute respiratory syndrome coronavirus 2
NLTTRTQLPPAYTNS

SEQ ID NO: 75 - protein, 15 aa, Severe acute respiratory syndrome coronavirus 2
RTQLPPAYTNSFTRG

SEQ ID NO: 76 - protein, 15 aa, Severe acute respiratory syndrome coronavirus 2
PPAYTNSFTRGVYYP

SEQ ID NO: 77 - protein, 15 aa, Severe acute respiratory syndrome coronavirus 2
TNSFTRGVYYPDKVF

SEQ ID NO: 78 - protein, 15 aa, Severe acute respiratory syndrome coronavirus 2
TRGVYYPDKVFRSSV

SEQ ID NO: 79 - protein, 15 aa, Severe acute respiratory syndrome coronavirus 2
YYPDKVFRSSVLHST

SEQ ID NO: 80 - protein, 15 aa, Severe acute respiratory syndrome coronavirus 2
KVFRSSVLHSTQDLF

SEQ ID NO: 81 - protein, 15 aa, Severe acute respiratory syndrome coronavirus 2
SSVLHSTQDLFLPFF

SEQ ID NO: 82 - protein, 15 aa, Severe acute respiratory syndrome coronavirus 2
HSTQDLFLPFFSNVT

SEQ ID NO: 83 - protein, 15 aa, Severe acute respiratory syndrome coronavirus 2
DLFLPFFSNVTWFHA

SEQ ID NO: 84 - protein, 15 aa, Severe acute respiratory syndrome coronavirus 2
PFFSNVTWFHAIHVS

SEQ ID NO: 85 - protein, 15 aa, Severe acute respiratory syndrome coronavirus 2
NVTWFHAIHVSGTNG

SEQ ID NO: 86 - protein, 15 aa, Severe acute respiratory syndrome coronavirus 2
FHAIHVSGTNGTKRF

SEQ ID NO: 87 - protein, 15 aa, Severe acute respiratory syndrome coronavirus 2
HVSGTNGTKRFDNPV

SEQ ID NO: 88 - protein, 15 aa, Severe acute respiratory syndrome coronavirus 2
TNGTKRFDNPVLPFN

SEQ ID NO: 89 - protein, 15 aa, Severe acute respiratory syndrome coronavirus 2
KRFDNPVLPFNDGVY

SEQ ID NO: 90 - protein, 15 aa, Severe acute respiratory syndrome coronavirus 2
NPVLPFNDGVYFAST

SEQ ID NO: 91 - protein, 15 aa, Severe acute respiratory syndrome coronavirus 2
PFNDGVYFASTEKSN

SEQ ID NO: 92 - protein, 15 aa, Severe acute respiratory syndrome coronavirus 2
GVYFASTEKSNIIRG

SEQ ID NO: 93 - protein, 15 aa, Severe acute respiratory syndrome coronavirus 2
ASTEKSNIIRGWIFG

SEQ ID NO: 94 - protein, 15 aa, Severe acute respiratory syndrome coronavirus 2
KSNIIRGWIFGTTLD

SEQ ID NO: 95 - protein, 15 aa, Severe acute respiratory syndrome coronavirus 2
IRGWIFGTTLDSKTQ

SEQ ID NO: 96 - protein, 15 aa, Severe acute respiratory syndrome coronavirus 2
IFGTTLDSKTQSLLI

SEQ ID NO: 97 - protein, 15 aa, Severe acute respiratory syndrome coronavirus 2
TLDSKTQSLLIVNNA

SEQ ID NO: 98 - protein, 15 aa, Severe acute respiratory syndrome coronavirus 2
KTQSLLIVNNATNVV

SEQ ID NO: 99 - protein, 15 aa, Severe acute respiratory syndrome coronavirus 2
LLIVNNATNVVIKVC

SEQ ID NO: 100 - protein, 15 aa, Severe acute respiratory syndrome coronavirus 2
NNATNVVIKVCEFQF

SEQ ID NO: 101 - protein, 15 aa, Severe acute respiratory syndrome coronavirus 2
NVVIKVCEFQFCNDP

SEQ ID NO: 102 - protein, 15 aa, Severe acute respiratory syndrome coronavirus 2
KVCEFQFCNDPFLGV

SEQ ID NO: 103 - protein, 15 aa, Severe acute respiratory syndrome coronavirus 2
FQFCNDPFLGVYYHK

SEQ ID NO: 104 - protein, 15 aa, Severe acute respiratory syndrome coronavirus 2
NDPFLGVYYHKNNKS

SEQ ID NO: 105 - protein, 15 aa, Severe acute respiratory syndrome coronavirus 2
LGVYYHKNNKSWMES

SEQ ID NO: 106 - protein, 15 aa, Severe acute respiratory syndrome coronavirus 2
YHKNNKSWMESEFRV

SEQ ID NO: 107 - protein, 15 aa, Severe acute respiratory syndrome coronavirus 2
NKSWMESEFRVYSSA

SEQ ID NO: 108 - protein, 15 aa, Severe acute respiratory syndrome coronavirus 2
MESEFRVYSSANNCT

SEQ ID NO: 109 - protein, 15 aa, Severe acute respiratory syndrome coronavirus 2
FRVYSSANNCTFEYV

SEQ ID NO: 110 - protein, 15 aa, Severe acute respiratory syndrome coronavirus 2
SSANNCTFEYVSQPF

SEQ ID NO: 111 - protein, 15 aa, Severe acute respiratory syndrome coronavirus 2
NCTFEYVSQPFLMDL

SEQ ID NO: 112 - protein, 15 aa, Severe acute respiratory syndrome coronavirus 2
EYVSQPFLMDLEGKQ

SEQ ID NO: 113 - protein, 15 aa, Severe acute respiratory syndrome coronavirus 2
QPFLMDLEGKQGNFK

SEQ ID NO: 114 - protein, 15 aa, Severe acute respiratory syndrome coronavirus 2
MDLEGKQGNFKNLRE

SEQ ID NO: 115 - protein, 15 aa, Severe acute respiratory syndrome coronavirus 2
GKQGNFKNLREFVFK

SEQ ID NO: 116 - protein, 15 aa, Severe acute respiratory syndrome coronavirus 2
NFKNLREFVFKNIDG

SEQ ID NO: 117 - protein, 15 aa, Severe acute respiratory syndrome coronavirus 2
LREFVFKNIDGYFKI

SEQ ID NO: 118 - protein, 15 aa, Severe acute respiratory syndrome coronavirus 2
VFKNIDGYFKIYSKH

SEQ ID NO: 119 - protein, 15 aa, Severe acute respiratory syndrome coronavirus 2
IDGYFKIYSKHTPIN

SEQ ID NO: 120 - protein, 15 aa, Severe acute respiratory syndrome coronavirus 2
FKIYSKHTPINLVRD

SEQ ID NO: 121 - protein, 15 aa, Severe acute respiratory syndrome coronavirus 2
SKHTPINLVRDLPQG

SEQ ID NO: 122 - protein, 15 aa, Severe acute respiratory syndrome coronavirus 2
PINLVRDLPQGFSAL

SEQ ID NO: 123 - protein, 15 aa, Severe acute respiratory syndrome coronavirus 2
VRDLPQGFSALEPLV

SEQ ID NO: 124 - protein, 15 aa, Severe acute respiratory syndrome coronavirus 2
PQGFSALEPLVDLPI

SEQ ID NO: 125 - protein, 15 aa, Severe acute respiratory syndrome coronavirus 2
SALEPLVDLPIGINI

SEQ ID NO: 126 - protein, 15 aa, Severe acute respiratory syndrome coronavirus 2
PLVDLPIGINITRFQ

SEQ ID NO: 127 - protein, 15 aa, Severe acute respiratory syndrome coronavirus 2
LPIGINITRFQTLLA

SEQ ID NO: 128 - protein, 15 aa, Severe acute respiratory syndrome coronavirus 2
INITRFQTLLALHRS

SEQ ID NO: 129 - protein, 15 aa, Severe acute respiratory syndrome coronavirus 2
RFQTLLALHRSYLTP

SEQ ID NO: 130 - protein, 15 aa, Severe acute respiratory syndrome coronavirus 2
LLALHRSYLTPGDSS

SEQ ID NO: 131 - protein, 15 aa, Severe acute respiratory syndrome coronavirus 2
HRSYLTPGDSSSGWT

SEQ ID NO: 132 - protein, 15 aa, Severe acute respiratory syndrome coronavirus 2
LTPGDSSSGWTAGAA

SEQ ID NO: 133 - protein, 15 aa, Severe acute respiratory syndrome coronavirus 2
DSSSGWTAGAAAYYV

SEQ ID NO: 134 - protein, 15 aa, Severe acute respiratory syndrome coronavirus 2
GWTAGAAAYYVGYLQ

SEQ ID NO: 135 - protein, 15 aa, Severe acute respiratory syndrome coronavirus 2
GAAAYYVGYLQPRTF

SEQ ID NO: 136 - protein, 15 aa, Severe acute respiratory syndrome coronavirus 2
YYVGYLQPRTFLLKY

SEQ ID NO: 137 - protein, 15 aa, Severe acute respiratory syndrome coronavirus 2
YLQPRTFLLKYNENG

SEQ ID NO: 138 - protein, 15 aa, Severe acute respiratory syndrome coronavirus 2
RTFLLKYNENGTITD

SEQ ID NO: 139 - protein, 15 aa, Severe acute respiratory syndrome coronavirus 2
LKYNENGTITDAVDC

SEQ ID NO: 140 - protein, 15 aa, Severe acute respiratory syndrome coronavirus 2
ENGTITDAVDCALDP

SEQ ID NO: 141 - protein, 15 aa, Severe acute respiratory syndrome coronavirus 2
ITDAVDCALDPLSET

SEQ ID NO: 142 - protein, 15 aa, Severe acute respiratory syndrome coronavirus 2
VDCALDPLSETKCTL

SEQ ID NO: 143 - protein, 15 aa, Severe acute respiratory syndrome coronavirus 2
LDPLSETKCTLKSFT

SEQ ID NO: 144 - protein, 15 aa, Severe acute respiratory syndrome coronavirus 2
SETKCTLKSFTVEKG

SEQ ID NO: 145 - protein, 15 aa, Severe acute respiratory syndrome coronavirus 2
CTLKSFTVEKGIYQT

SEQ ID NO: 146 - protein, 15 aa, Severe acute respiratory syndrome coronavirus 2
SFTVEKGIYQTSNFR

SEQ ID NO: 147 - protein, 15 aa, Severe acute respiratory syndrome coronavirus 2
EKGIYQTSNFRVQPT

SEQ ID NO: 148 - protein, 15 aa, Severe acute respiratory syndrome coronavirus 2
YQTSNFRVQPTESIV

SEQ ID NO: 149 - protein, 15 aa, Severe acute respiratory syndrome coronavirus 2
NFRVQPTESIVRFPN

SEQ ID NO: 150 - protein, 15 aa, Severe acute respiratory syndrome coronavirus 2
QPTESIVRFPNITNL

SEQ ID NO: 151 - protein, 15 aa, Severe acute respiratory syndrome coronavirus 2
SIVRFPNITNLCPFG

SEQ ID NO: 152 - protein, 15 aa, Severe acute respiratory syndrome coronavirus 2
FPNITNLCPFGEVFN

SEQ ID NO: 153 - protein, 15 aa, Severe acute respiratory syndrome coronavirus 2
TNLCPFGEVFNATRF

SEQ ID NO: 154 - protein, 15 aa, Severe acute respiratory syndrome coronavirus 2
PFGEVFNATRFASVY

SEQ ID NO: 155 - protein, 15 aa, Severe acute respiratory syndrome coronavirus 2
VFNATRFASVYAWNR

SEQ ID NO: 156 - protein, 15 aa, Severe acute respiratory syndrome coronavirus 2
TRFASVYAWNRKRIS

SEQ ID NO: 157 - protein, 15 aa, Severe acute respiratory syndrome coronavirus 2
SVYAWNRKRISNCVA

SEQ ID NO: 158 - protein, 15 aa, Severe acute respiratory syndrome coronavirus 2
WNRKRISNCVADYSV

SEQ ID NO: 159 - protein, 15 aa, Severe acute respiratory syndrome coronavirus 2
RISNCVADYSVLYNS

SEQ ID NO: 160 - protein, 15 aa, Severe acute respiratory syndrome coronavirus 2
CVADYSVLYNSASFS

SEQ ID NO: 161 - protein, 15 aa, Severe acute respiratory syndrome coronavirus 2
YSVLYNSASFSTFKC

SEQ ID NO: 162 - protein, 15 aa, Severe acute respiratory syndrome coronavirus 2
YNSASFSTFKCYGVS

SEQ ID NO: 163 - protein, 15 aa, Severe acute respiratory syndrome coronavirus 2
SFSTFKCYGVSPTKL

SEQ ID NO: 164 - protein, 15 aa, Severe acute respiratory syndrome coronavirus 2
FKCYGVSPTKLNDLC

SEQ ID NO: 165 - protein, 15 aa, Severe acute respiratory syndrome coronavirus 2
GVSPTKLNDLCFTNV

SEQ ID NO: 166 - protein, 15 aa, Severe acute respiratory syndrome coronavirus 2
TKLNDLCFTNVYADS

SEQ ID NO: 167 - protein, 15 aa, Severe acute respiratory syndrome coronavirus 2
DLCFTNVYADSFVIR

SEQ ID NO: 168 - protein, 15 aa, Severe acute respiratory syndrome coronavirus 2
TNVYADSFVIRGDEV

SEQ ID NO: 169 - protein, 15 aa, Severe acute respiratory syndrome coronavirus 2
ADSFVIRGDEVRQIA

SEQ ID NO: 170 - protein, 15 aa, Severe acute respiratory syndrome coronavirus 2
VIRGDEVRQIAPGQT

SEQ ID NO: 171 - protein, 15 aa, Severe acute respiratory syndrome coronavirus 2
DEVRQIAPGQTGKIA

SEQ ID NO: 172 - protein, 15 aa, Severe acute respiratory syndrome coronavirus 2
QIAPGQTGKIADYNY

SEQ ID NO: 173 - protein, 15 aa, Severe acute respiratory syndrome coronavirus 2
GQTGKIADYNYKLPD

SEQ ID NO: 174 - protein, 15 aa, Severe acute respiratory syndrome coronavirus 2
KIADYNYKLPDDFTG

SEQ ID NO: 175 - protein, 15 aa, Severe acute respiratory syndrome coronavirus 2
YNYKLPDDFTGCVIA

SEQ ID NO: 176 - protein, 15 aa, Severe acute respiratory syndrome coronavirus 2
LPDDFTGCVIAWNSN

SEQ ID NO: 177 - protein, 15 aa, Severe acute respiratory syndrome coronavirus 2
FTGCVIAWNSNNLDS

SEQ ID NO: 178 - protein, 15 aa, Severe acute respiratory syndrome coronavirus 2
VIAWNSNNLDSKVGG

SEQ ID NO: 179 - protein, 15 aa, Severe acute respiratory syndrome coronavirus 2
NSNNLDSKVGGNYNY

SEQ ID NO: 180 - protein, 15 aa, Severe acute respiratory syndrome coronavirus 2
LDSKVGGNYNYLYRL

SEQ ID NO: 181 - protein, 15 aa, Severe acute respiratory syndrome coronavirus 2
VGGNYNYLYRLFRKS

SEQ ID NO: 182 - protein, 15 aa, Severe acute respiratory syndrome coronavirus 2
YNYLYRLFRKSNLKP

SEQ ID NO: 183 - protein, 15 aa, Severe acute respiratory syndrome coronavirus 2
YRLFRKSNLKPFERD

SEQ ID NO: 184 - protein, 15 aa, Severe acute respiratory syndrome coronavirus 2
RKSNLKPFERDISTE

SEQ ID NO: 185 - protein, 15 aa, Severe acute respiratory syndrome coronavirus 2
LKPFERDISTEIYQA

SEQ ID NO: 186 - protein, 15 aa, Severe acute respiratory syndrome coronavirus 2
ERDISTEIYQAGSTP

SEQ ID NO: 187 - protein, 15 aa, Severe acute respiratory syndrome coronavirus 2
STEIYQAGSTPCNGV

SEQ ID NO: 188 - protein, 15 aa, Severe acute respiratory syndrome coronavirus 2
YQAGSTPCNGVEGFN

SEQ ID NO: 189 - protein, 15 aa, Severe acute respiratory syndrome coronavirus 2
STPCNGVEGFNCYFP

SEQ ID NO: 190 - protein, 15 aa, Severe acute respiratory syndrome coronavirus 2
NGVEGFNCYFPLQSY

SEQ ID NO: 191 - protein, 15 aa, Severe acute respiratory syndrome coronavirus 2
GFNCYFPLQSYGFQP

SEQ ID NO: 192 - protein, 15 aa, Severe acute respiratory syndrome coronavirus 2
YFPLQSYGFQPTNGV

SEQ ID NO: 193 - protein, 15 aa, Severe acute respiratory syndrome coronavirus 2
QSYGFQPTNGVGYQP

SEQ ID NO: 194 - protein, 15 aa, Severe acute respiratory syndrome coronavirus 2
FQPTNGVGYQPYRVV

SEQ ID NO: 195 - protein, 15 aa, Severe acute respiratory syndrome coronavirus 2
NGVGYQPYRVVVLSF

SEQ ID NO: 196 - protein, 15 aa, Severe acute respiratory syndrome coronavirus 2
YQPYRVVVLSFELLH

SEQ ID NO: 197 - protein, 15 aa, Severe acute respiratory syndrome coronavirus 2
RVVVLSFELLHAPAT

SEQ ID NO: 198 - protein, 15 aa, Severe acute respiratory syndrome coronavirus 2
LSFELLHAPATVCGP

SEQ ID NO: 199 - protein, 15 aa, Severe acute respiratory syndrome coronavirus 2
LLHAPATVCGPKKST

SEQ ID NO: 200 - protein, 15 aa, Severe acute respiratory syndrome coronavirus 2
PATVCGPKKSTNLVK

SEQ ID NO: 201 - protein, 15 aa, Severe acute respiratory syndrome coronavirus 2
CGPKKSTNLVKNKCV

SEQ ID NO: 202 - protein, 15 aa, Severe acute respiratory syndrome coronavirus 2
KSTNLVKNKCVNFNF

SEQ ID NO: 203 - protein, 15 aa, Severe acute respiratory syndrome coronavirus 2
LVKNKCVNFNFNGLT

SEQ ID NO: 204 - protein, 15 aa, Severe acute respiratory syndrome coronavirus 2
KCVNFNFNGLTGTGV

SEQ ID NO: 205 - protein, 15 aa, Severe acute respiratory syndrome coronavirus 2
FNFNGLTGTGVLTES

SEQ ID NO: 206 - protein, 15 aa, Severe acute respiratory syndrome coronavirus 2
GLTGTGVLTESNKKF

SEQ ID NO: 207 - protein, 15 aa, Severe acute respiratory syndrome coronavirus 2
TGVLTESNKKFLPFQ

SEQ ID NO: 208 - protein, 15 aa, Severe acute respiratory syndrome coronavirus 2
TESNKKFLPFQQFGR

SEQ ID NO: 209 - protein, 15 aa, Severe acute respiratory syndrome coronavirus 2
KKFLPFQQFGRDIAD

SEQ ID NO: 210 - protein, 15 aa, Severe acute respiratory syndrome coronavirus 2
PFQQFGRDIADTTDA

SEQ ID NO: 211 - protein, 15 aa, Severe acute respiratory syndrome coronavirus 2
FGRDIADTTDAVRDP

SEQ ID NO: 212 - protein, 15 aa, Severe acute respiratory syndrome coronavirus 2
IADTTDAVRDPQTLE

SEQ ID NO: 213 - protein, 15 aa, Severe acute respiratory syndrome coronavirus 2
TDAVRDPQTLEILDI

SEQ ID NO: 214 - protein, 15 aa, Severe acute respiratory syndrome coronavirus 2
RDPQTLEILDITPCS

SEQ ID NO: 215 - protein, 15 aa, Severe acute respiratory syndrome coronavirus 2
TLEILDITPCSFGGV

SEQ ID NO: 216 - protein, 15 aa, Severe acute respiratory syndrome coronavirus 2
LDITPCSFGGVSVIT

SEQ ID NO: 217 - protein, 15 aa, Severe acute respiratory syndrome coronavirus 2
PCSFGGVSVITPGTN

SEQ ID NO: 218 - protein, 15 aa, Severe acute respiratory syndrome coronavirus 2
GGVSVITPGTNTSNQ

SEQ ID NO: 219 - protein, 15 aa, Severe acute respiratory syndrome coronavirus 2
VITPGTNTSNQVAVL

SEQ ID NO: 220 - protein, 15 aa, Severe acute respiratory syndrome coronavirus 2
GTNTSNQVAVLYQDV

SEQ ID NO: 221 - protein, 15 aa, Severe acute respiratory syndrome coronavirus 2
SNQVAVLYQDVNCTE

SEQ ID NO: 222 - protein, 15 aa, Severe acute respiratory syndrome coronavirus 2
AVLYQDVNCTEVPVA

SEQ ID NO: 223 - protein, 15 aa, Severe acute respiratory syndrome coronavirus 2
QDVNCTEVPVAIHAD

SEQ ID NO: 224 - protein, 15 aa, Severe acute respiratory syndrome coronavirus 2
CTEVPVAIHADQLTP

SEQ ID NO: 225 - protein, 15 aa, Severe acute respiratory syndrome coronavirus 2
PVAIHADQLTPTWRV

SEQ ID NO: 226 - protein, 15 aa, Severe acute respiratory syndrome coronavirus 2
HADQLTPTWRVYSTG

SEQ ID NO: 227 - protein, 15 aa, Severe acute respiratory syndrome coronavirus 2
LTPTWRVYSTGSNVF

SEQ ID NO: 228 - protein, 15 aa, Severe acute respiratory syndrome coronavirus 2
WRVYSTGSNVFQTRA

SEQ ID NO: 229 - protein, 15 aa, Severe acute respiratory syndrome coronavirus 2
STGSNVFQTRAGCLI

SEQ ID NO: 230 - protein, 15 aa, Severe acute respiratory syndrome coronavirus 2
NVFQTRAGCLIGAEH

SEQ ID NO: 231 - protein, 15 aa, Severe acute respiratory syndrome coronavirus 2
TRAGCLIGAEHVNNS

SEQ ID NO: 232 - protein, 15 aa, Severe acute respiratory syndrome coronavirus 2
CLIGAEHVNNSYECD

SEQ ID NO: 233 - protein, 15 aa, Severe acute respiratory syndrome coronavirus 2
AEHVNNSYECDIPIG

SEQ ID NO: 234 - protein, 15 aa, Severe acute respiratory syndrome coronavirus 2
NNSYECDIPIGAGIC

SEQ ID NO: 235 - protein, 15 aa, Severe acute respiratory syndrome coronavirus 2
ECDIPIGAGICASYQ

SEQ ID NO: 236 - protein, 15 aa, Severe acute respiratory syndrome coronavirus 2
PIGAGICASYQTQTN

SEQ ID NO: 237 - protein, 15 aa, Severe acute respiratory syndrome coronavirus 2
GICASYQTQTNSPRR

SEQ ID NO: 238 - protein, 15 aa, Severe acute respiratory syndrome coronavirus 2
SYQTQTNSPRRARSV

SEQ ID NO: 239 - protein, 15 aa, Severe acute respiratory syndrome coronavirus 2
QTNSPRRARSVASQS

SEQ ID NO: 240 - protein, 15 aa, Severe acute respiratory syndrome coronavirus 2
PRRARSVASQSIIAY

SEQ ID NO: 241 - protein, 15 aa, Severe acute respiratory syndrome coronavirus 2
RSVASQSIIAYTMSL

SEQ ID NO: 242 - protein, 15 aa, Severe acute respiratory syndrome coronavirus 2
SQSIIAYTMSLGAEN

SEQ ID NO: 243 - protein, 15 aa, Severe acute respiratory syndrome coronavirus 2
IAYTMSLGAENSVAY

SEQ ID NO: 244 - protein, 15 aa, Severe acute respiratory syndrome coronavirus 2
MSLGAENSVAYSNNS

SEQ ID NO: 245 - protein, 15 aa, Severe acute respiratory syndrome coronavirus 2
AENSVAYSNNSIAIP

SEQ ID NO: 246 - protein, 15 aa, Severe acute respiratory syndrome coronavirus 2
VAYSNNSIAIPTNFT

SEQ ID NO: 247 - protein, 15 aa, Severe acute respiratory syndrome coronavirus 2
NNSIAIPTNFTISVT

SEQ ID NO: 248 - protein, 15 aa, Severe acute respiratory syndrome coronavirus 2
AIPTNFTISVTTEIL

SEQ ID NO: 249 - protein, 15 aa, Severe acute respiratory syndrome coronavirus 2
NFTISVTTEILPVSM

SEQ ID NO: 250 - protein, 15 aa, Severe acute respiratory syndrome coronavirus 2
SVTTEILPVSMTKTS

SEQ ID NO: 251 - protein, 15 aa, Severe acute respiratory syndrome coronavirus 2
EILPVSMTKTSVDCT

SEQ ID NO: 252 - protein, 15 aa, Severe acute respiratory syndrome coronavirus 2
VSMTKTSVDCTMYIC

SEQ ID NO: 253 - protein, 15 aa, Severe acute respiratory syndrome coronavirus 2
KTSVDCTMYICGDST

SEQ ID NO: 254 - protein, 15 aa, Severe acute respiratory syndrome coronavirus 2
DCTMYICGDSTECSN

SEQ ID NO: 255 - protein, 15 aa, Severe acute respiratory syndrome coronavirus 2
YICGDSTECSNLLLQ

SEQ ID NO: 256 - protein, 15 aa, Severe acute respiratory syndrome coronavirus 2
DSTECSNLLLQYGSF

SEQ ID NO: 257 - protein, 15 aa, Severe acute respiratory syndrome coronavirus 2
CSNLLLQYGSFCTQL

SEQ ID NO: 258 - protein, 15 aa, Severe acute respiratory syndrome coronavirus 2
LLQYGSFCTQLNRAL

SEQ ID NO: 259 - protein, 15 aa, Severe acute respiratory syndrome coronavirus 2
GSFCTQLNRALTGIA

SEQ ID NO: 260 - protein, 15 aa, Severe acute respiratory syndrome coronavirus 2
TQLNRALTGIAVEQD

SEQ ID NO: 261 - protein, 15 aa, Severe acute respiratory syndrome coronavirus 2
RALTGIAVEQDKNTQ

SEQ ID NO: 262 - protein, 15 aa, Severe acute respiratory syndrome coronavirus 2
GIAVEQDKNTQEVFA

SEQ ID NO: 263 - protein, 15 aa, Severe acute respiratory syndrome coronavirus 2
EQDKNTQEVFAQVKQ

SEQ ID NO: 264 - protein, 15 aa, Severe acute respiratory syndrome coronavirus 2
NTQEVFAQVKQIYKT

SEQ ID NO: 265 - protein, 15 aa, Severe acute respiratory syndrome coronavirus 2
VFAQVKQIYKTPPIK

SEQ ID NO: 266 - protein, 15 aa, Severe acute respiratory syndrome coronavirus 2
VKQIYKTPPIKDFGG

SEQ ID NO: 267 - protein, 15 aa, Severe acute respiratory syndrome coronavirus 2
YKTPPIKDFGGFNFS

SEQ ID NO: 268 - protein, 15 aa, Severe acute respiratory syndrome coronavirus 2
PIKDFGGFNFSQILP

SEQ ID NO: 269 - protein, 15 aa, Severe acute respiratory syndrome coronavirus 2
FGGFNFSQILPDPSK

SEQ ID NO: 270 - protein, 15 aa, Severe acute respiratory syndrome coronavirus 2
NFSQILPDPSKPSKR

SEQ ID NO: 271 - protein, 15 aa, Severe acute respiratory syndrome coronavirus 2
ILPDPSKPSKRSFIE

SEQ ID NO: 272 - protein, 15 aa, Severe acute respiratory syndrome coronavirus 2
PSKPSKRSFIEDLLF

SEQ ID NO: 273 - protein, 15 aa, Severe acute respiratory syndrome coronavirus 2
SKRSFIEDLLFNKVT

SEQ ID NO: 274 - protein, 15 aa, Severe acute respiratory syndrome coronavirus 2
FIEDLLFNKVTLADA

SEQ ID NO: 275 - protein, 15 aa, Severe acute respiratory syndrome coronavirus 2
LLFNKVTLADAGFIK

SEQ ID NO: 276 - protein, 15 aa, Severe acute respiratory syndrome coronavirus 2
KVTLADAGFIKQYGD

SEQ ID NO: 277 - protein, 15 aa, Severe acute respiratory syndrome coronavirus 2
ADAGFIKQYGDCLGD

SEQ ID NO: 278 - protein, 15 aa, Severe acute respiratory syndrome coronavirus 2
FIKQYGDCLGDIAAR

SEQ ID NO: 279 - protein, 15 aa, Severe acute respiratory syndrome coronavirus 2
YGDCLGDIAARDLIC

SEQ ID NO: 280 - protein, 15 aa, Severe acute respiratory syndrome coronavirus 2
LGDIAARDLICAQKF

SEQ ID NO: 281 - protein, 15 aa, Severe acute respiratory syndrome coronavirus 2
AARDLICAQKFNGLT

SEQ ID NO: 282 - protein, 15 aa, Severe acute respiratory syndrome coronavirus 2
LICAQKFNGLTVLPP

SEQ ID NO: 283 - protein, 15 aa, Severe acute respiratory syndrome coronavirus 2
QKFNGLTVLPPLLTD

SEQ ID NO: 284 - protein, 15 aa, Severe acute respiratory syndrome coronavirus 2
GLTVLPPLLTDEMIA

SEQ ID NO: 285 - protein, 15 aa, Severe acute respiratory syndrome coronavirus 2
LPPLLTDEMIAQYTS

SEQ ID NO: 286 - protein, 15 aa, Severe acute respiratory syndrome coronavirus 2
LTDEMIAQYTSALLA

SEQ ID NO: 287 - protein, 15 aa, Severe acute respiratory syndrome coronavirus 2
MIAQYTSALLAGTIT

SEQ ID NO: 288 - protein, 15 aa, Severe acute respiratory syndrome coronavirus 2
YTSALLAGTITSGWT

SEQ ID NO: 289 - protein, 15 aa, Severe acute respiratory syndrome coronavirus 2
LLAGTITSGWTFGAG

SEQ ID NO: 290 - protein, 15 aa, Severe acute respiratory syndrome coronavirus 2
TITSGWTFGAGAALQ

SEQ ID NO: 291 - protein, 15 aa, Severe acute respiratory syndrome coronavirus 2
GWTFGAGAALQIPFA

SEQ ID NO: 292 - protein, 15 aa, Severe acute respiratory syndrome coronavirus 2
GAGAALQIPFAMQMA

SEQ ID NO: 293 - protein, 15 aa, Severe acute respiratory syndrome coronavirus 2
ALQIPFAMQMAYRFN

SEQ ID NO: 294 - protein, 15 aa, Severe acute respiratory syndrome coronavirus 2
PFAMQMAYRFNGIGV

SEQ ID NO: 295 - protein, 15 aa, Severe acute respiratory syndrome coronavirus 2
QMAYRFNGIGVTQNV

SEQ ID NO: 296 - protein, 15 aa, Severe acute respiratory syndrome coronavirus 2
RFNGIGVTQNVLYEN

SEQ ID NO: 297 - protein, 15 aa, Severe acute respiratory syndrome coronavirus 2
IGVTQNVLYENQKLI

SEQ ID NO: 298 - protein, 15 aa, Severe acute respiratory syndrome coronavirus 2
QNVLYENQKLIANQF

SEQ ID NO: 299 - protein, 15 aa, Severe acute respiratory syndrome coronavirus 2
YENQKLIANQFNSAI

SEQ ID NO: 300 - protein, 15 aa, Severe acute respiratory syndrome coronavirus 2
KLIANQFNSAIGKIQ

SEQ ID NO: 301 - protein, 15 aa, Severe acute respiratory syndrome coronavirus 2
NQFNSAIGKIQDSLS

SEQ ID NO: 302 - protein, 15 aa, Severe acute respiratory syndrome coronavirus 2
SAIGKIQDSLSSTAS

SEQ ID NO: 303 - protein, 15 aa, Severe acute respiratory syndrome coronavirus 2
KIQDSLSSTASALGK

SEQ ID NO: 304 - protein, 15 aa, Severe acute respiratory syndrome coronavirus 2
SLSSTASALGKLQDV

SEQ ID NO: 305 - protein, 15 aa, Severe acute respiratory syndrome coronavirus 2
TASALGKLQDVVNQN

SEQ ID NO: 306 - protein, 15 aa, Severe acute respiratory syndrome coronavirus 2
LGKLQDVVNQNAQAL

SEQ ID NO: 307 - protein, 15 aa, Severe acute respiratory syndrome coronavirus 2
QDVVNQNAQALNTLV

SEQ ID NO: 308 - protein, 15 aa, Severe acute respiratory syndrome coronavirus 2
NQNAQALNTLVKQLS

SEQ ID NO: 309 - protein, 15 aa, Severe acute respiratory syndrome coronavirus 2
QALNTLVKQLSSNFG

SEQ ID NO: 310 - protein, 15 aa, Severe acute respiratory syndrome coronavirus 2
TLVKQLSSNFGAISS

SEQ ID NO: 311 - protein, 15 aa, Severe acute respiratory syndrome coronavirus 2
QLSSNFGAISSVLND

SEQ ID NO: 312 - protein, 15 aa, Severe acute respiratory syndrome coronavirus 2
NFGAISSVLNDILSR

SEQ ID NO: 313 - protein, 15 aa, Severe acute respiratory syndrome coronavirus 2
ISSVLNDILSRLDKV

SEQ ID NO: 314 - protein, 15 aa, Severe acute respiratory syndrome coronavirus 2
LNDILSRLDKVEAEV

SEQ ID NO: 315 - protein, 15 aa, Severe acute respiratory syndrome coronavirus 2
LSRLDKVEAEVQIDR

SEQ ID NO: 316 - protein, 15 aa, Severe acute respiratory syndrome coronavirus 2
DKVEAEVQIDRLITG

SEQ ID NO: 317 - protein, 15 aa, Severe acute respiratory syndrome coronavirus 2
AEVQIDRLITGRLQS

SEQ ID NO: 318 - protein, 15 aa, Severe acute respiratory syndrome coronavirus 2
IDRLITGRLQSLQTY

SEQ ID NO: 319 - protein, 15 aa, Severe acute respiratory syndrome coronavirus 2
ITGRLQSLQTYVTQQ

SEQ ID NO: 320 - protein, 15 aa, Severe acute respiratory syndrome coronavirus 2
LQSLQTYVTQQLIRA

SEQ ID NO: 321 - protein, 15 aa, Severe acute respiratory syndrome coronavirus 2
QTYVTQQLIRAAEIR

SEQ ID NO: 322 - protein, 15 aa, Severe acute respiratory syndrome coronavirus 2
TQQLIRAAEIRASAN

SEQ ID NO: 323 - protein, 15 aa, Severe acute respiratory syndrome coronavirus 2
IRAAEIRASANLAAT

SEQ ID NO: 324 - protein, 15 aa, Severe acute respiratory syndrome coronavirus 2
EIRASANLAATKMSE

SEQ ID NO: 325 - protein, 15 aa, Severe acute respiratory syndrome coronavirus 2
SANLAATKMSECVLG

SEQ ID NO: 326 - protein, 15 aa, Severe acute respiratory syndrome coronavirus 2
AATKMSECVLGQSKR

SEQ ID NO: 327 - protein, 15 aa, Severe acute respiratory syndrome coronavirus 2
MSECVLGQSKRVDFC

SEQ ID NO: 328 - protein, 15 aa, Severe acute respiratory syndrome coronavirus 2
VLGQSKRVDFCGKGY

SEQ ID NO: 329 - protein, 15 aa, Severe acute respiratory syndrome coronavirus 2
SKRVDFCGKGYHLMS

SEQ ID NO: 330 - protein, 15 aa, Severe acute respiratory syndrome coronavirus 2
DFCGKGYHLMSFPQS

SEQ ID NO: 331 - protein, 15 aa, Severe acute respiratory syndrome coronavirus 2
KGYHLMSFPQSAPHG

SEQ ID NO: 332 - protein, 15 aa, Severe acute respiratory syndrome coronavirus 2
LMSFPQSAPHGVVFL

SEQ ID NO: 333 - protein, 15 aa, Severe acute respiratory syndrome coronavirus 2
PQSAPHGVVFLHVTY

SEQ ID NO: 334 - protein, 15 aa, Severe acute respiratory syndrome coronavirus 2
PHGVVFLHVTYVPAQ

SEQ ID NO: 335 - protein, 15 aa, Severe acute respiratory syndrome coronavirus 2
VFLHVTYVPAQEKNF

SEQ ID NO: 336 - protein, 15 aa, Severe acute respiratory syndrome coronavirus 2
VTYVPAQEKNFTTAP

SEQ ID NO: 337 - protein, 15 aa, Severe acute respiratory syndrome coronavirus 2
PAQEKNFTTAPAICH

SEQ ID NO: 338 - protein, 15 aa, Severe acute respiratory syndrome coronavirus 2
KNFTTAPAICHDGKA

SEQ ID NO: 339 - protein, 15 aa, Severe acute respiratory syndrome coronavirus 2
TAPAICHDGKAHFPR

SEQ ID NO: 340 - protein, 15 aa, Severe acute respiratory syndrome coronavirus 2
ICHDGKAHFPREGVF

SEQ ID NO: 341 - protein, 15 aa, Severe acute respiratory syndrome coronavirus 2
GKAHFPREGVFVSNG

SEQ ID NO: 342 - protein, 15 aa, Severe acute respiratory syndrome coronavirus 2
FPREGVFVSNGTHWF

SEQ ID NO: 343 - protein, 15 aa, Severe acute respiratory syndrome coronavirus 2
GVFVSNGTHWFVTQR

SEQ ID NO: 344 - protein, 15 aa, Severe acute respiratory syndrome coronavirus 2
SNGTHWFVTQRNFYE

SEQ ID NO: 345 - protein, 15 aa, Severe acute respiratory syndrome coronavirus 2
HWFVTQRNFYEPQII

SEQ ID NO: 346 - protein, 15 aa, Severe acute respiratory syndrome coronavirus 2
TQRNFYEPQIITTDN

SEQ ID NO: 347 - protein, 15 aa, Severe acute respiratory syndrome coronavirus 2
FYEPQIITTDNTFVS

SEQ ID NO: 348 - protein, 15 aa, Severe acute respiratory syndrome coronavirus 2
QIITTDNTFVSGNCD

SEQ ID NO: 349 - protein, 15 aa, Severe acute respiratory syndrome coronavirus 2
TDNTFVSGNCDVVIG

SEQ ID NO: 350 - protein, 15 aa, Severe acute respiratory syndrome coronavirus 2
FVSGNCDVVIGIVNN

SEQ ID NO: 351 - protein, 15 aa, Severe acute respiratory syndrome coronavirus 2
NCDVVIGIVNNTVYD

SEQ ID NO: 352 - protein, 15 aa, Severe acute respiratory syndrome coronavirus 2
VIGIVNNTVYDPLQP

SEQ ID NO: 353 - protein, 15 aa, Severe acute respiratory syndrome coronavirus 2
VNNTVYDPLQPELDS

SEQ ID NO: 354 - protein, 15 aa, Severe acute respiratory syndrome coronavirus 2
VYDPLQPELDSFKEE

SEQ ID NO: 355 - protein, 15 aa, Severe acute respiratory syndrome coronavirus 2
LQPELDSFKEELDKY

SEQ ID NO: 356 - protein, 15 aa, Severe acute respiratory syndrome coronavirus 2
LDSFKEELDKYFKNH

SEQ ID NO: 357 - protein, 15 aa, Severe acute respiratory syndrome coronavirus 2
KEELDKYFKNHTSPD

SEQ ID NO: 358 - protein, 15 aa, Severe acute respiratory syndrome coronavirus 2
DKYFKNHTSPDVDLG

SEQ ID NO: 359 - protein, 15 aa, Severe acute respiratory syndrome coronavirus 2
KNHTSPDVDLGDISG

SEQ ID NO: 360 - protein, 15 aa, Severe acute respiratory syndrome coronavirus 2
SPDVDLGDISGINAS

SEQ ID NO: 361 - protein, 15 aa, Severe acute respiratory syndrome coronavirus 2
DLGDISGINASVVNI

SEQ ID NO: 362 - protein, 15 aa, Severe acute respiratory syndrome coronavirus 2
ISGINASVVNIQKEI

SEQ ID NO: 363 - protein, 15 aa, Severe acute respiratory syndrome coronavirus 2
NASVVNIQKEIDRLN

SEQ ID NO: 364 - protein, 15 aa, Severe acute respiratory syndrome coronavirus 2
VNIQKEIDRLNEVAK

SEQ ID NO: 365 - protein, 15 aa, Severe acute respiratory syndrome coronavirus 2
KEIDRLNEVAKNLNE

SEQ ID NO: 366 - protein, 15 aa, Severe acute respiratory syndrome coronavirus 2
RLNEVAKNLNESLID

SEQ ID NO: 367 - protein, 15 aa, Severe acute respiratory syndrome coronavirus 2
VAKNLNESLIDLQEL

SEQ ID NO: 368 - protein, 15 aa, Severe acute respiratory syndrome coronavirus 2
LNESLIDLQELGKYE

SEQ ID NO: 369 - protein, 15 aa, Severe acute respiratory syndrome coronavirus 2
LIDLQELGKYEQYIK

SEQ ID NO: 370 - protein, 15 aa, Severe acute respiratory syndrome coronavirus 2
QELGKYEQYIKWPWY

SEQ ID NO: 371 - protein, 15 aa, Severe acute respiratory syndrome coronavirus 2
KYEQYIKWPWYIWLG

SEQ ID NO: 372 - protein, 15 aa, Severe acute respiratory syndrome coronavirus 2
YIKWPWYIWLGFIAG

SEQ ID NO: 373 - protein, 15 aa, Severe acute respiratory syndrome coronavirus 2
PWYIWLGFIAGLIAI

SEQ ID NO: 374 - protein, 15 aa, Severe acute respiratory syndrome coronavirus 2
WLGFIAGLIAIVMVT

SEQ ID NO: 375 - protein, 15 aa, Severe acute respiratory syndrome coronavirus 2
IAGLIAIVMVTIMLC

SEQ ID NO: 376 - protein, 15 aa, Severe acute respiratory syndrome coronavirus 2
IAIVMVTIMLCCMTS

SEQ ID NO: 377 - protein, 15 aa, Severe acute respiratory syndrome coronavirus 2
MVTIMLCCMTSCCSC

SEQ ID NO: 378 - protein, 15 aa, Severe acute respiratory syndrome coronavirus 2
MLCCMTSCCSCLKGC

SEQ ID NO: 379 - protein, 15 aa, Severe acute respiratory syndrome coronavirus 2
MTSCCSCLKGCCSCG

SEQ ID NO: 380 - protein, 15 aa, Severe acute respiratory syndrome coronavirus 2
CSCLKGCCSCGSCCK

SEQ ID NO: 381 - protein, 15 aa, Severe acute respiratory syndrome coronavirus 2
KGCCSCGSCCKFDED

SEQ ID NO: 382 - protein, 15 aa, Severe acute respiratory syndrome coronavirus 2
SCGSCCKFDEDDSEP

SEQ ID NO: 383 - protein, 15 aa, Severe acute respiratory syndrome coronavirus 2
CCKFDEDDSEPVLKG

SEQ ID NO: 384 - protein, 15 aa, Severe acute respiratory syndrome coronavirus 2
DEDDSEPVLKGVKLH

SEQ ID NO: 385 - protein, 13 aa, Severe acute respiratory syndrome coronavirus 2
SEPVLKGVKLHYT

SEQ ID NO: 386 - protein, 15 aa, Severe acute respiratory syndrome coronavirus 2
MSDNGPQNQRNAPRI

SEQ ID NO: 387 - protein, 15 aa, Severe acute respiratory syndrome coronavirus 2
GPQNQRNAPRITFGG

SEQ ID NO: 388 - protein, 15 aa, Severe acute respiratory syndrome coronavirus 2
QRNAPRITFGGPSDS

SEQ ID NO: 389 - protein, 15 aa, Severe acute respiratory syndrome coronavirus 2
PRITFGGPSDSTGSN

SEQ ID NO: 390 - protein, 15 aa, Severe acute respiratory syndrome coronavirus 2
FGGPSDSTGSNQNGE

SEQ ID NO: 391 - protein, 15 aa, Severe acute respiratory syndrome coronavirus 2
SDSTGSNQNGERSGA

SEQ ID NO: 392 - protein, 15 aa, Severe acute respiratory syndrome coronavirus 2
GSNQNGERSGARSKQ

SEQ ID NO: 393 - protein, 15 aa, Severe acute respiratory syndrome coronavirus 2
NGERSGARSKQRRPQ

SEQ ID NO: 394 - protein, 15 aa, Severe acute respiratory syndrome coronavirus 2
SGARSKQRRPQGLPN

SEQ ID NO: 395 - protein, 15 aa, Severe acute respiratory syndrome coronavirus 2
SKQRRPQGLPNNTAS

SEQ ID NO: 396 - protein, 15 aa, Severe acute respiratory syndrome coronavirus 2
RPQGLPNNTASWFTA

SEQ ID NO: 397 - protein, 15 aa, Severe acute respiratory syndrome coronavirus 2
LPNNTASWFTALTQH

SEQ ID NO: 398 - protein, 15 aa, Severe acute respiratory syndrome coronavirus 2
TASWFTALTQHGKED

SEQ ID NO: 399 - protein, 15 aa, Severe acute respiratory syndrome coronavirus 2
FTALTQHGKEDLKFP

SEQ ID NO: 400 - protein, 15 aa, Severe acute respiratory syndrome coronavirus 2
TQHGKEDLKFPRGQG

SEQ ID NO: 401 - protein, 15 aa, Severe acute respiratory syndrome coronavirus 2
KEDLKFPRGQGVPIN

SEQ ID NO: 402 - protein, 15 aa, Severe acute respiratory syndrome coronavirus 2
KFPRGQGVPINTNSS

SEQ ID NO: 403 - protein, 15 aa, Severe acute respiratory syndrome coronavirus 2
GQGVPINTNSSPDDQ

SEQ ID NO: 404 - protein, 15 aa, Severe acute respiratory syndrome coronavirus 2
PINTNSSPDDQIGYY

SEQ ID NO: 405 - protein, 15 aa, Severe acute respiratory syndrome coronavirus 2
NSSPDDQIGYYRRAT

SEQ ID NO: 406 - protein, 15 aa, Severe acute respiratory syndrome coronavirus 2
DDQIGYYRRATRRIR

SEQ ID NO: 407 - protein, 15 aa, Severe acute respiratory syndrome coronavirus 2
GYYRRATRRIRGGDG

SEQ ID NO: 408 - protein, 15 aa, Severe acute respiratory syndrome coronavirus 2
RATRRIRGGDGKMKD

SEQ ID NO: 409 - protein, 15 aa, Severe acute respiratory syndrome coronavirus 2
RIRGGDGKMKDLSPR

SEQ ID NO: 410 - protein, 15 aa, Severe acute respiratory syndrome coronavirus 2
GDGKMKDLSPRWYFY

SEQ ID NO: 411 - protein, 15 aa, Severe acute respiratory syndrome coronavirus 2
MKDLSPRWYFYYLGT

SEQ ID NO: 412 - protein, 15 aa, Severe acute respiratory syndrome coronavirus 2
SPRWYFYYLGTGPEA

SEQ ID NO: 413 - protein, 15 aa, Severe acute respiratory syndrome coronavirus 2
YFYYLGTGPEAGLPY

SEQ ID NO: 414 - protein, 15 aa, Severe acute respiratory syndrome coronavirus 2
LGTGPEAGLPYGANK

SEQ ID NO: 415 - protein, 15 aa, Severe acute respiratory syndrome coronavirus 2
PEAGLPYGANKDGII

SEQ ID NO: 416 - protein, 15 aa, Severe acute respiratory syndrome coronavirus 2
LPYGANKDGIIWVAT

SEQ ID NO: 417 - protein, 15 aa, Severe acute respiratory syndrome coronavirus 2
ANKDGIIWVATEGAL

SEQ ID NO: 418 - protein, 15 aa, Severe acute respiratory syndrome coronavirus 2
GIIWVATEGALNTPK

SEQ ID NO: 419 - protein, 15 aa, Severe acute respiratory syndrome coronavirus 2
VATEGALNTPKDHIG

SEQ ID NO: 420 - protein, 15 aa, Severe acute respiratory syndrome coronavirus 2
GALNTPKDHIGTRNP

SEQ ID NO: 421 - protein, 15 aa, Severe acute respiratory syndrome coronavirus 2
TPKDHIGTRNPANNA

SEQ ID NO: 422 - protein, 15 aa, Severe acute respiratory syndrome coronavirus 2
HIGTRNPANNAAIVL

SEQ ID NO: 423 - protein, 15 aa, Severe acute respiratory syndrome coronavirus 2
RNPANNAAIVLQLPQ

SEQ ID NO: 424 - protein, 15 aa, Severe acute respiratory syndrome coronavirus 2
NNAAIVLQLPQGTTL

SEQ ID NO: 425 - protein, 15 aa, Severe acute respiratory syndrome coronavirus 2
IVLQLPQGTTLPKGF

SEQ ID NO: 426 - protein, 15 aa, Severe acute respiratory syndrome coronavirus 2
LPQGTTLPKGFYAEG

SEQ ID NO: 427 - protein, 15 aa, Severe acute respiratory syndrome coronavirus 2
TTLPKGFYAEGSRGG

SEQ ID NO: 428 - protein, 15 aa, Severe acute respiratory syndrome coronavirus 2
KGFYAEGSRGGSQAS

SEQ ID NO: 429 - protein, 15 aa, Severe acute respiratory syndrome coronavirus 2
AEGSRGGSQASSRSS

SEQ ID NO: 430 - protein, 15 aa, Severe acute respiratory syndrome coronavirus 2
RGGSQASSRSSSRSR

SEQ ID NO: 431 - protein, 15 aa, Severe acute respiratory syndrome coronavirus 2
QASSRSSSRSRNSSR

SEQ ID NO: 432 - protein, 15 aa, Severe acute respiratory syndrome coronavirus 2
RSSSRSRNSSRNSTP

SEQ ID NO: 433 - protein, 15 aa, Severe acute respiratory syndrome coronavirus 2
RSRNSSRNSTPGSSR

SEQ ID NO: 434 - protein, 15 aa, Severe acute respiratory syndrome coronavirus 2
SSRNSTPGSSRGTSP

SEQ ID NO: 435 - protein, 15 aa, Severe acute respiratory syndrome coronavirus 2
STPGSSRGTSPARMA

SEQ ID NO: 436 - protein, 15 aa, Severe acute respiratory syndrome coronavirus 2
SSRGTSPARMAGNGG

SEQ ID NO: 437 - protein, 15 aa, Severe acute respiratory syndrome coronavirus 2
TSPARMAGNGGDAAL

SEQ ID NO: 438 - protein, 15 aa, Severe acute respiratory syndrome coronavirus 2
RMAGNGGDAALALLL

SEQ ID NO: 439 - protein, 15 aa, Severe acute respiratory syndrome coronavirus 2
NGGDAALALLLLDRL

SEQ ID NO: 440 - protein, 15 aa, Severe acute respiratory syndrome coronavirus 2
AALALLLLDRLNQLE

SEQ ID NO: 441 - protein, 15 aa, Severe acute respiratory syndrome coronavirus 2
LLLLDRLNQLESKMS

SEQ ID NO: 442 - protein, 15 aa, Severe acute respiratory syndrome coronavirus 2
DRLNQLESKMSGKGQ

SEQ ID NO: 443 - protein, 15 aa, Severe acute respiratory syndrome coronavirus 2
QLESKMSGKGQQQQG

SEQ ID NO: 444 - protein, 15 aa, Severe acute respiratory syndrome coronavirus 2
KMSGKGQQQQGQTVT

SEQ ID NO: 445 - protein, 15 aa, Severe acute respiratory syndrome coronavirus 2
KGQQQQGQTVTKKSA

SEQ ID NO: 446 - protein, 15 aa, Severe acute respiratory syndrome coronavirus 2
QQGQTVTKKSAAEAS

SEQ ID NO: 447 - protein, 15 aa, Severe acute respiratory syndrome coronavirus 2
TVTKKSAAEASKKPR

SEQ ID NO: 448 - protein, 15 aa, Severe acute respiratory syndrome coronavirus 2
KSAAEASKKPRQKRT

SEQ ID NO: 449 - protein, 15 aa, Severe acute respiratory syndrome coronavirus 2
EASKKPRQKRTATKA

SEQ ID NO: 450 - protein, 15 aa, Severe acute respiratory syndrome coronavirus 2
KPRQKRTATKAYNVT

SEQ ID NO: 451 - protein, 15 aa, Severe acute respiratory syndrome coronavirus 2
KRTATKAYNVTQAFG

SEQ ID NO: 452 - protein, 15 aa, Severe acute respiratory syndrome coronavirus 2
TKAYNVTQAFGRRGP

SEQ ID NO: 453 - protein, 15 aa, Severe acute respiratory syndrome coronavirus 2
NVTQAFGRRGPEQTQ

SEQ ID NO: 454 - protein, 15 aa, Severe acute respiratory syndrome coronavirus 2
AFGRRGPEQTQGNFG

SEQ ID NO: 455 - protein, 15 aa, Severe acute respiratory syndrome coronavirus 2
RGPEQTQGNFGDQEL

SEQ ID NO: 456 - protein, 15 aa, Severe acute respiratory syndrome coronavirus 2
QTQGNFGDQELIRQG

SEQ ID NO: 457 - protein, 15 aa, Severe acute respiratory syndrome coronavirus 2
NFGDQELIRQGTDYK

SEQ ID NO: 458 - protein, 15 aa, Severe acute respiratory syndrome coronavirus 2
QELIRQGTDYKHWPQ

SEQ ID NO: 459 - protein, 15 aa, Severe acute respiratory syndrome coronavirus 2
RQGTDYKHWPQIAQF

SEQ ID NO: 460 - protein, 15 aa, Severe acute respiratory syndrome coronavirus 2
DYKHWPQIAQFAPSA

SEQ ID NO: 461 - protein, 15 aa, Severe acute respiratory syndrome coronavirus 2
WPQIAQFAPSASAFF

SEQ ID NO: 462 - protein, 15 aa, Severe acute respiratory syndrome coronavirus 2
AQFAPSASAFFGMSR

SEQ ID NO: 463 - protein, 15 aa, Severe acute respiratory syndrome coronavirus 2
PSASAFFGMSRIGME

SEQ ID NO: 464 - protein, 15 aa, Severe acute respiratory syndrome coronavirus 2
AFFGMSRIGMEVTPS

SEQ ID NO: 465 - protein, 15 aa, Severe acute respiratory syndrome coronavirus 2
MSRIGMEVTPSGTWL

SEQ ID NO: 466 - protein, 15 aa, Severe acute respiratory syndrome coronavirus 2
GMEVTPSGTWLTYTG

SEQ ID NO: 467 - protein, 15 aa, Severe acute respiratory syndrome coronavirus 2
TPSGTWLTYTGAIKL

SEQ ID NO: 468 - protein, 15 aa, Severe acute respiratory syndrome coronavirus 2
TWLTYTGAIKLDDKD

SEQ ID NO: 469 - protein, 15 aa, Severe acute respiratory syndrome coronavirus 2
YTGAIKLDDKDPNFK

SEQ ID NO: 470 - protein, 15 aa, Severe acute respiratory syndrome coronavirus 2
IKLDDKDPNFKDQVI

SEQ ID NO: 471 - protein, 15 aa, Severe acute respiratory syndrome coronavirus 2
DKDPNFKDQVILLNK

SEQ ID NO: 472 - protein, 15 aa, Severe acute respiratory syndrome coronavirus 2
NFKDQVILLNKHIDA

SEQ ID NO: 473 - protein, 15 aa, Severe acute respiratory syndrome coronavirus 2
QVILLNKHIDAYKTF

SEQ ID NO: 474 - protein, 15 aa, Severe acute respiratory syndrome coronavirus 2
LNKHIDAYKTFPPTE

SEQ ID NO: 475 - protein, 15 aa, Severe acute respiratory syndrome coronavirus 2
IDAYKTFPPTEPKKD

SEQ ID NO: 476 - protein, 15 aa, Severe acute respiratory syndrome coronavirus 2
KTFPPTEPKKDKKKK

SEQ ID NO: 477 - protein, 15 aa, Severe acute respiratory syndrome coronavirus 2
PTEPKKDKKKKADET

SEQ ID NO: 478 - protein, 15 aa, Severe acute respiratory syndrome coronavirus 2
KKDKKKKADETQALP

SEQ ID NO: 479 - protein, 15 aa, Severe acute respiratory syndrome coronavirus 2
KKKADETQALPQRQK

SEQ ID NO: 480 - protein, 15 aa, Severe acute respiratory syndrome coronavirus 2
DETQALPQRQKKQQT

SEQ ID NO: 481 - protein, 15 aa, Severe acute respiratory syndrome coronavirus 2
ALPQRQKKQQTVTLL

SEQ ID NO: 482 - protein, 15 aa, Severe acute respiratory syndrome coronavirus 2
RQKKQQTVTLLPAAD

SEQ ID NO: 483 - protein, 15 aa, Severe acute respiratory syndrome coronavirus 2
QQTVTLLPAADLDDF

SEQ ID NO: 484 - protein, 15 aa, Severe acute respiratory syndrome coronavirus 2
TLLPAADLDDFSKQL

SEQ ID NO: 485 - protein, 15 aa, Severe acute respiratory syndrome coronavirus 2
AADLDDFSKQLQQSM

SEQ ID NO: 486 - protein, 15 aa, Severe acute respiratory syndrome coronavirus 2
DDFSKQLQQSMSSAD

SEQ ID NO: 487 - protein, 15 aa, Severe acute respiratory syndrome coronavirus 2
KQLQQSMSSADSTQA

SEQ ID NO: 488 - protein, 15 aa, Coronavirus OC43
LNKPRQKRSPNKQCT

SEQ ID NO: 489 - protein, 15 aa, Coronavirus NL63
KPRWKRVPTREENVI

SEQ ID NO: 490 - protein, 15 aa, Severe acute respiratory syndrome coronavirus 2
PINLVRDLPQGFWAL

SEQ ID NO: 491 - protein, 15 aa, Severe acute respiratory syndrome coronavirus 2
KPRQKRTATKAYNVI

SEQ ID NO: 492 - protein, 15 aa, Severe acute respiratory syndrome coronavirus 2
SRMLSYYKLGASQRV

SEQ ID NO: 493 - protein, 15 aa, Coronavirus
LRGHLRIAGHHLGRC

SEQ ID NO: 494 - protein, 14 aa, Coronavirus
RGHLRMAGHPLGRC

SEQ ID NO: 495 - protein, 12 aa, Coronavirus
RGHLYIQGVKLG

SEQ ID NO: 496 - protein, 13 aa, Coronavirus
GHLKIAGMHFGAC

SEQ ID NO: 497 - protein, 15 aa, Coronavirus
LRIAGHHLGRCDIKD

SEQ ID NO: 498 - protein, 15 aa, Coronavirus
LRMAGHPLGRCDIKD

SEQ ID NO: 499 - protein, 15 aa, Coronavirus
SRTLSYYKLGASQRV

SEQ ID NO: 500 - protein, 15 aa, Coronavirus
SRTLSYYKLGASQRV

SEQ ID NO: 501 - protein, 15 aa, Coronavirus
SYYKLGASQRVAGDS

SEQ ID NO: 502 - protein, 15 aa, Coronavirus
SYYKLGASQRVGTDS

SEQ ID NO: 503 - protein, 15 aa, Coronavirus
LGASQRVAGDSGFAA

SEQ ID NO: 504 - protein, 15 aa, Coronavirus
LGASQRVGTDSGFAA

SEQ ID NO: 505 - protein, 14 aa, Coronavirus
  MISC_FEATURE (5)..(5): X at position 5 is an alignment gap or any amino acid.
LGASXVTEDVKFAA

SEQ ID NO: 506 - protein, 15 aa, Coronavirus
KPRQKRTATKAYNVT

SEQ ID NO: 507 - protein, 15 aa, Coronavirus
KPRQKRTATKQYNVT

SEQ ID NO: 508 - protein, 10 aa, Coronavirus
KPRQKRSPNK

SEQ ID NO: 509 - protein, 14 aa, Coronavirus
KPRWKRVPTREENV

SEQ ID NO: 510 - protein, 12 aa, Coronavirus
RHKRVATKSFNV

SEQ ID NO: 511 - protein, 15 aa, Coronavirus
AFFGMSRIGMEVTPS

SEQ ID NO: 512 - protein, 15 aa, Coronavirus
AFFGMSRIGMEVTPS

SEQ ID NO: 513 - protein, 15 aa, Coronavirus
MSRIGMEVTPSGTWL

SEQ ID NO: 514 - protein, 15 aa, Coronavirus
MSRIGMEVTPSGTWL

SEQ ID NO: 515 - protein, 15 aa, Coronavirus
GMEVTPSGTWLTYTG

SEQ ID NO: 516 - protein, 13 aa, Coronavirus
GMEVTPSGTWLTY

SEQ ID NO: 517 - protein, 16 aa, Coronavirus
SKHTPINLVRDLPQFG

SEQ ID NO: 518 - protein, 10 aa, Coronavirus
  MISC_FEATURE (6)..(6): X at position 6 is an alignment gap or any amino acid.
PANIVXLPQG

SEQ ID NO: 519 - protein, 11 aa, Coronavirus
PIDVVRDLPSG

SEQ ID NO: 520 - protein, 15 aa, Coronavirus
PINLVRDLPQGFSAL

SEQ ID NO: 521 - protein, 15 aa, Coronavirus
  MISC_FEATURE (6)..(6): X at position 6 is an alignment gap or any amino acid.
PIDVVRDLPSGFNTL

SEQ ID NO: 522 - protein, 10 aa, Coronavirus
  misc_feature (6)..(6): Xaa can be any naturally occurring amino acid
PANIVXLPQG

SEQ ID NO: 523 - protein, 15 aa, Coronavirus
YNYLYRLFRKSNLKP

SEQ ID NO: 524 - protein, 15 aa, Coronavirus
YNYKYRYLRHGKLRP